(19) United States
(12) Patent Application Publication
(10) Pub. No.: US 2020/0199681 A1
(43) Pub. Date: Jun. 25, 2020

(54) MDA-7 CANCER THERAPIES AND METHODS OF DETECTING BIOMOLECULES

(71) Applicant: VIRGINIA COMMONWEATLH UNIVERSITY, Richmond, VA (US)

(72) Inventors: Paul B. FISHER, Henrico, VA (US); Anjan K. PRADHAN, Richmond, VA (US); Praveen BHOOPATHI, Richmond, VA (US); Swadesh DAS, Richmond, VA (US); Luni EMDAD, Richmond, VA (US)

(21) Appl. No.: 16/349,493

(22) PCT Filed: Nov. 14, 2017
(86) PCT No.: PCT/US2017/061527

§ 371 Date: May 13, 2019

Related U.S. Application Data
(60) Provisional application No. 62/421,484, filed on Nov. 14, 2016.

(51) Int. Cl. C12Q 1/6886; A61K 31/7088; A61K 38/17; C12Q 1/686

(52) U.S. Cl. CPC C12Q 1/6886; A61K 31/7088; A61K 38/1712; C12Q 1/686; C12Q 2531/113; C12Q 2600/158; C12Q 2600/178; C12Q 2561/113; C12Q 2600/106

(57) ABSTRACT

Provided herein are, inter alia, methods of detecting levels of miR-221 and beclin-1 in patients undergoing treatment for miR-221- and/or beclin-1-associated diseases (e.g., cancer, inflammatory disease, infectious disease, autoimmune disease, cardiovascular disease). The methods provided herein are useful, inter alia, to monitor and determine treatment efficacy by determining (detecting) levels of miR-221, beclin-1, a combination thereof or of molecules downstream of the miR-221 or beclin-1 signaling pathways, in patients receiving, having received or to be received MDA-7 treatment.

### CROSS-REFERENCES TO RELATED APPLICATIONS

This application claims the benefit of U.S. Provisional Application No. 62/421,484, filed Nov. 14, 2016, which is incorporated herein by reference in entirety and for all purposes

### STATEMENT AS TO RIGHTS TO INVENTIONS MADE UNDER FEDERALLY SPONSORED RESEARCH AND DEVELOPMENT

This invention was made with government support under grant nos. CA058236, NS047463, CA016059, CA097318, CA108520, P01 CA104177, and CA16059 awarded by the National Institutes of Health, and grant no. W81XWH-14-1-0409 awarded by the Department of Defense. The government has certain rights in the invention.

### BACKGROUND

Melanoma differentiation associated gene-7/Interleukin-24 (MDA-7/IL-24) displays broad spectrum anti-cancer activity without harming normal cells or tissues. The mechanism by which MDA-7 induces anti-cancer activity, however, is largely unknown.

MicroRNAs (miRNAs) play a central role in regulating different normal and pathological pathways, including development and cancer, respectively. MicroRNA-221 (miR-221) has been shown to be significantly upregulated in different diseases, including in different cancers, where it acts to degrade tumor suppressors. Thus, miR-221 is a promising target for the treatment of diseases, such as cancers, that show aberrant (e.g., upregulated) expression of miR-221.

Beclin-1, the mammalian homologue of Atg6 of yeast, is a promoter of autophagy. Expression of beclin-1 is altered in different disease states. In several types of cancer, aberrant mRNA/protein expression of beclin-1 has been observed. The underlying mechanism of this altered expression of beclin-1 is unknown. Beclin-1 is thus a promising therapeutic target for treatment of diseases, such as cancers, that show aberrant (e.g., downregulated) expression of beclin-1.

Disclosed herein are, inter alia, solutions to these and other needs in the art.

### BRIEF SUMMARY

In an aspect is provided a method of detecting a miR-221 level in a cancer patient, wherein the cancer patient has received a MDA-7 treatment, the method including: (i) obtaining a post-treatment biological sample from the cancer patient; and (ii) detecting a post-treatment level of miR-221 in the post-treatment biological sample.

In an aspect is provided a method of detecting a beclin-1 level in a cancer patient, wherein the cancer patient has received a MDA-7 treatment, the method including: (i) obtaining a post-treatment biological sample from the cancer patient; and (ii) detecting a post-treatment level of beclin-1 in the post-treatment biological sample.

In an aspect is provided a method of treating cancer in a subject in need thereof, wherein the subject has a cancer expressing miR-221 and not expressing MDA-7, the method including administering to the subject an effective amount of MDA-7.

In an aspect is provided a method of treating cancer in a subject in need thereof, wherein the subject has a cancer not expressing beclin-1 and not expressing MDA-7, the method including administering to the subject an effective amount of MDA-7.

In an aspect is provided a method of inhibiting cancer-associated angiogenesis in a subject in need thereof, the method including administering to the subject an effective amount of MDA-7.

In an aspect is provided a method of treating an autoimmune disease in a subject in need thereof, the method including administering to the subject an effective amount of MDA-7.

In an aspect is provided a method of treating an infectious disease in a subject in need thereof, the method including administering to the subject an effective amount of MDA-7.

In an aspect is provided a method of treating an inflammatory disease in a subject in need thereof, the method including administering to the subject an effective amount of MDA-7.

In an aspect is provided a method of treating a cardiovascular disease in a subject in need thereof, the method including administering to the subject an effective amount of MDA-7.

### BRIEF DESCRIPTION OF THE DRAWINGS

FIGS. 1A-1C. MDA-7/IL-24 regulates miR-221. FIG. 1A. MDA-MB-231 cells were infected with either Ad.null or Ad.mda-7. Seventy-two hours after infection miRNA fractions were isolated and real time PCR was done using different taqman probes, i.e., miR-221 or miR-222. RNU44 was used as an endogenous control. FIG. 1B. MDA-MB-231 cells were infected with increasing doses of Ad.mda-7 (500 vp, 1000 vp and 2000 vp per cell). Protein lysates were prepared at 72 hours after infection and Western blotting was done to check the levels of MDA-7/IL-24 and EF1α (loading control) (upper panel). miRNA fractions were also isolated at 72 hours after infection and real time PCR was done to check the level of miR-221 (middle panel). MTT assays were done to verify the inhibition of proliferation by mda-7/IL-24 (bottom panel). FIG. 1C. The down regulation of miR-221 by mda-7/IL-24 was temporal as confirmed by a time point kinetics study. MTT assays were done to check the effect of mda-7/IL-24 on the proliferation of cells. The level of MDA-7/IL-24 protein was checked by western blotting.

FIGS. 2A-2D. MDA-7/IL-24 down regulates miR-221 in diverse cancer cell lines. FIG. 2A. Different breast cancer cells were infected with Ad.null or Ad.mda-7 (2000 vp/cell) for 72 hours. RQ-PCR was performed to check the level of miR-221. FIG. 2B. Indicated cells were infected with Ad.null or Ad.mda-7 (2000 vp/cell) for 72 hours. RQ-PCR was performed to check the level of miR-221. FIG. 2C. A549 and DU-145 cells were treated with His-MDA-7. RQ-PCR was performed to check the level of miR-221. FIG. 2D. A549 cells were transfected with IL-20R2 or IL-22R1 and treated with His-MDA-7. RQ-PCR was performed to check the level of miR-221.

FIGS. 3A-3E. Over expression of miR-221 can rescue cells from mda-7/IL-24-mediated cell death. FIG. 3A. MDA-MB-231 cells were transfected with pCDNA3.1 (vector) or miR-221 and then treated with Ad.null or Ad.mda-7. After 72 hours cells were stained with Annexin-V and then analyzed by flow cytometer. FIG. 3B. Cells were treated as in FIG. 3A and

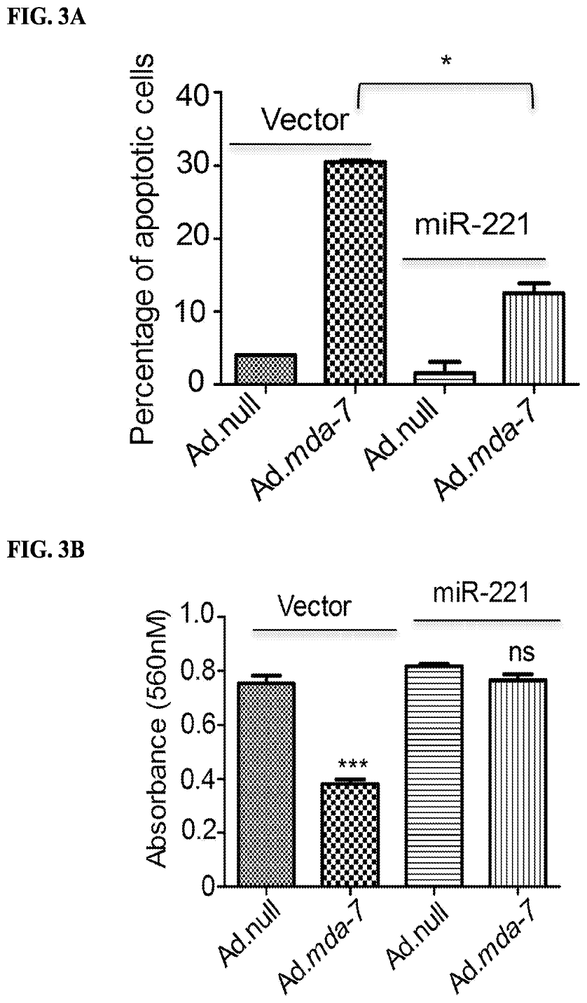

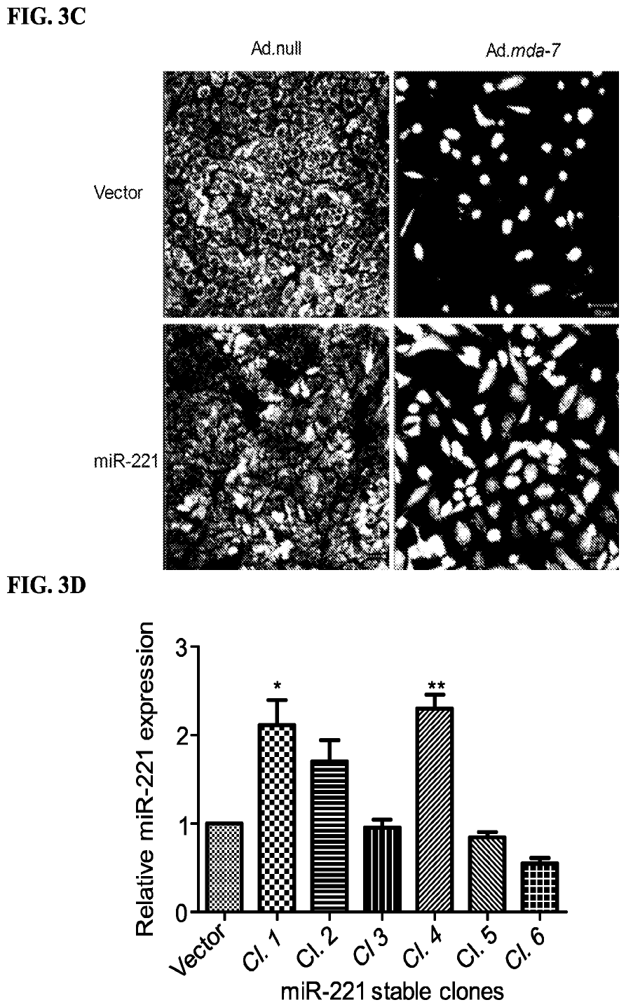

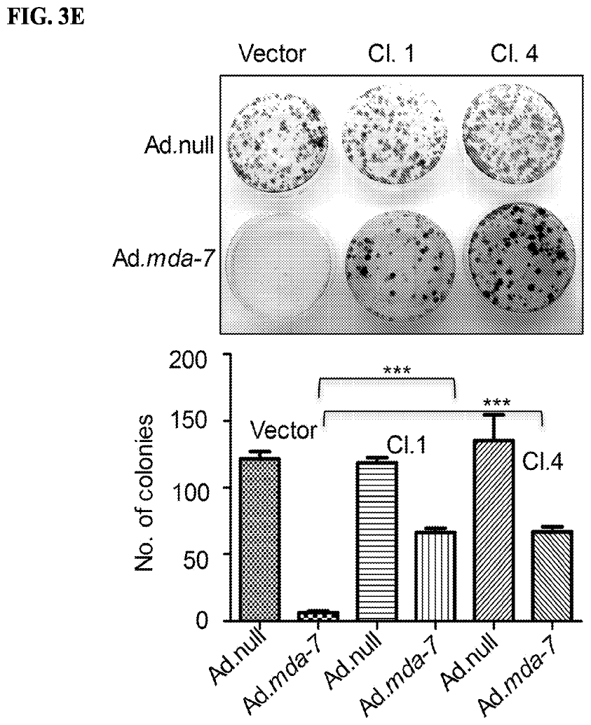

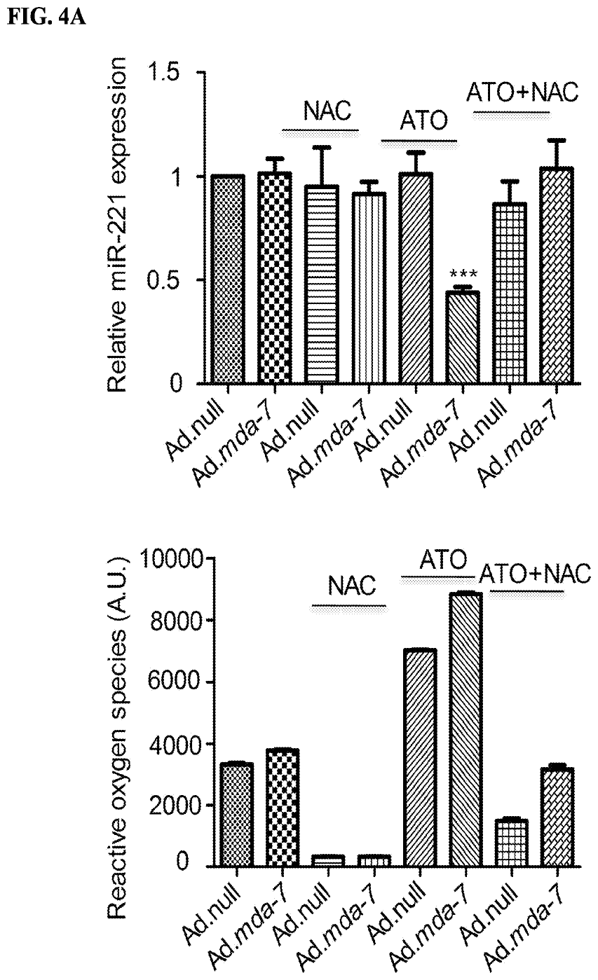

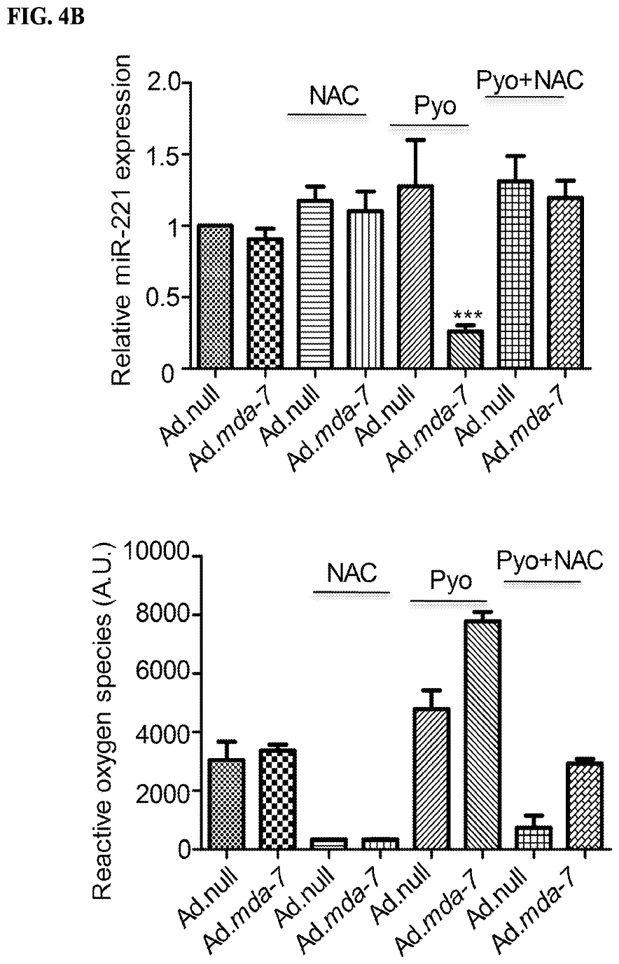

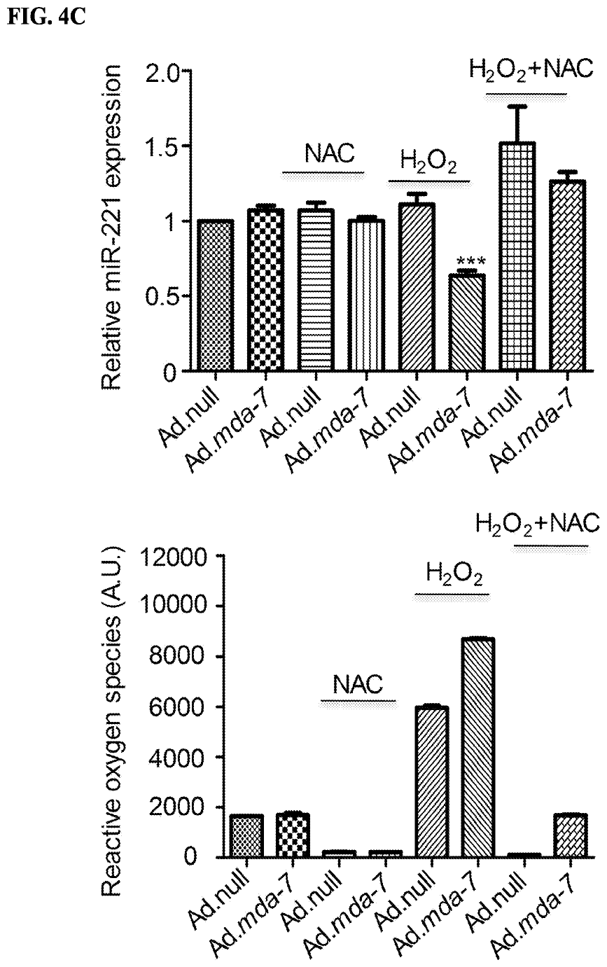

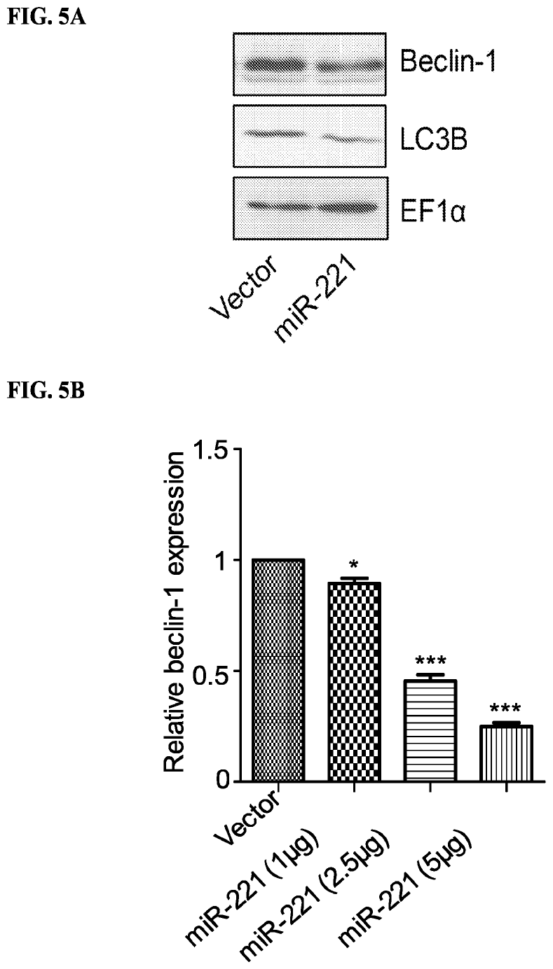

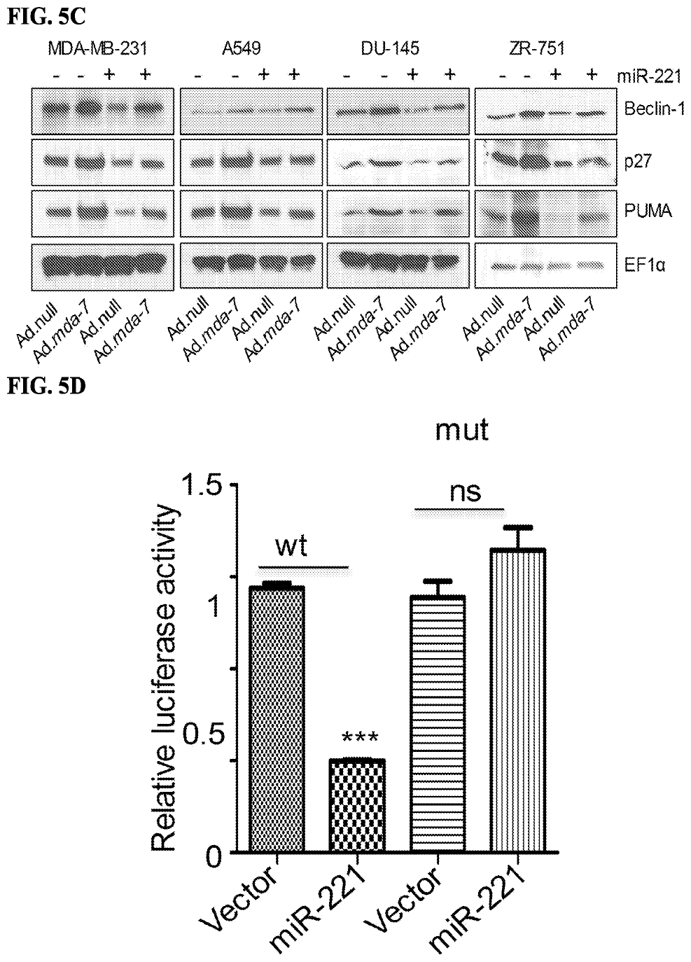

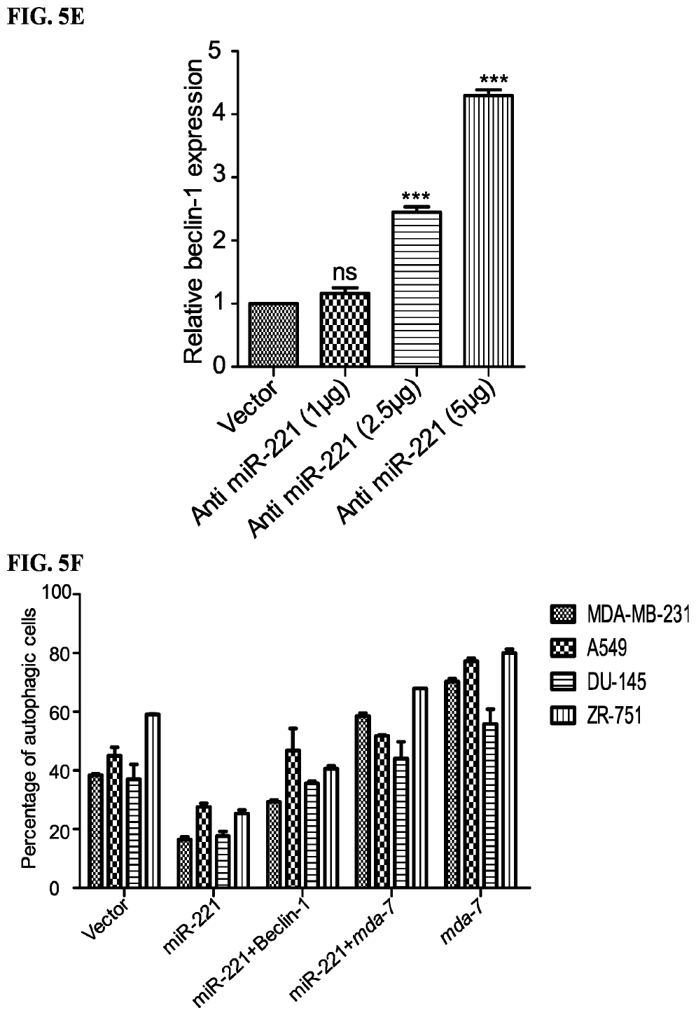

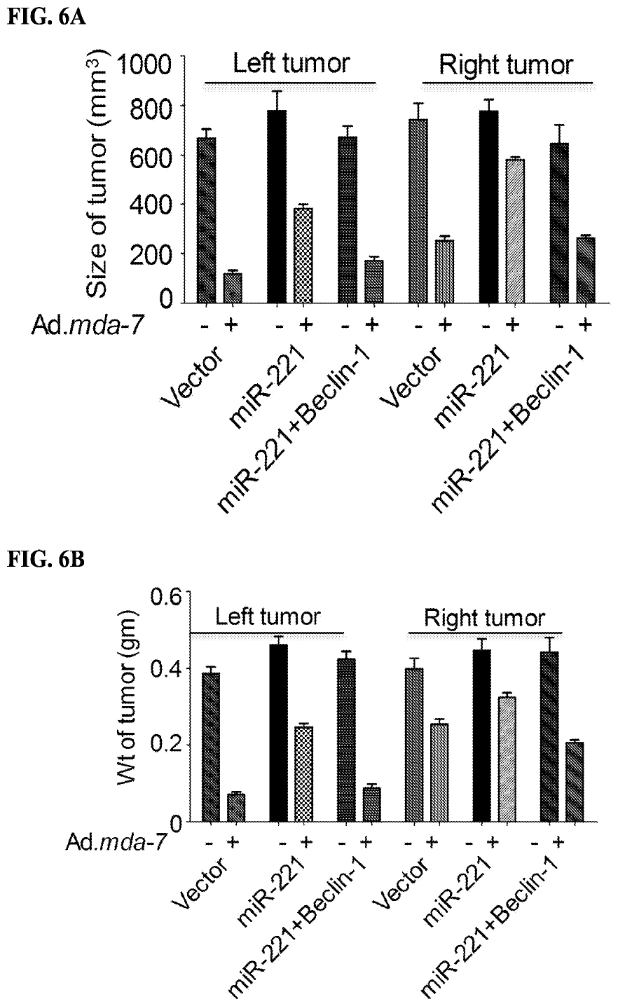

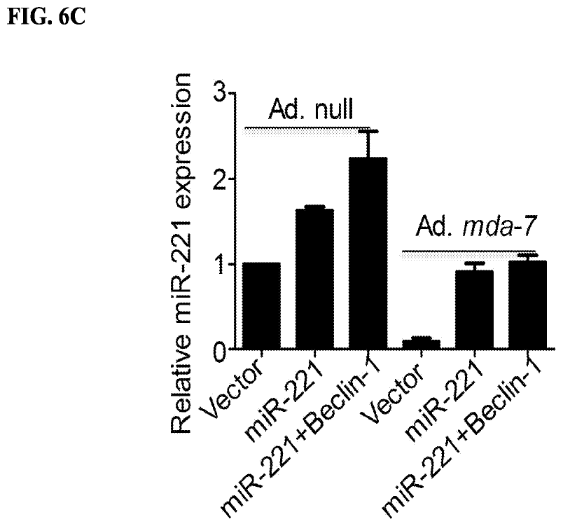

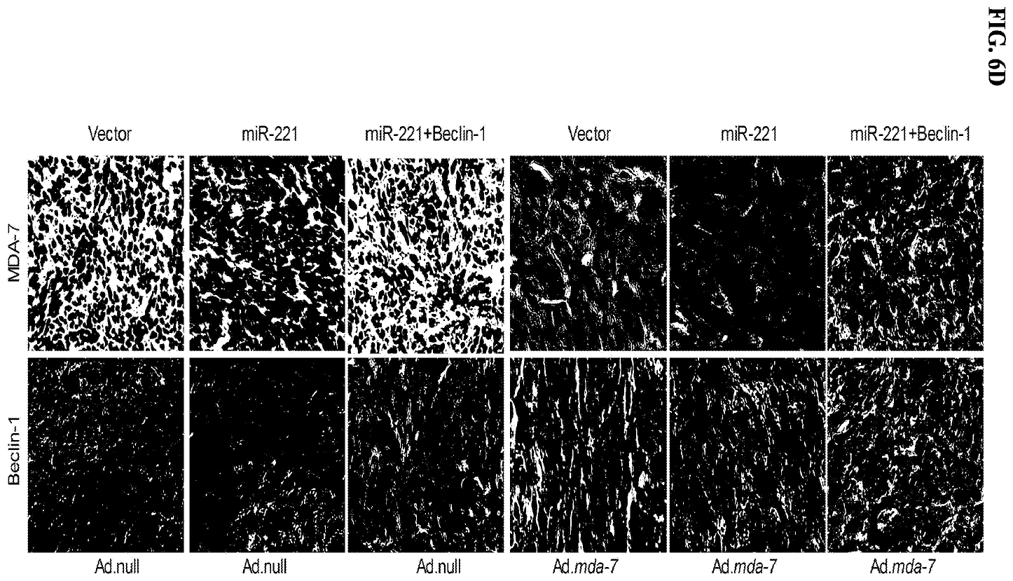

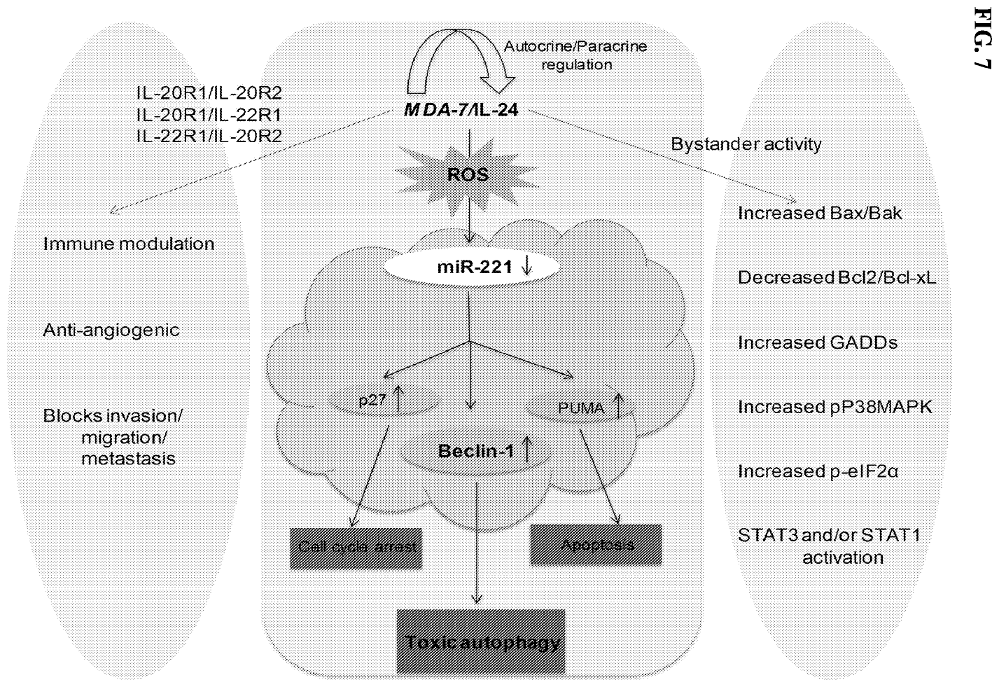

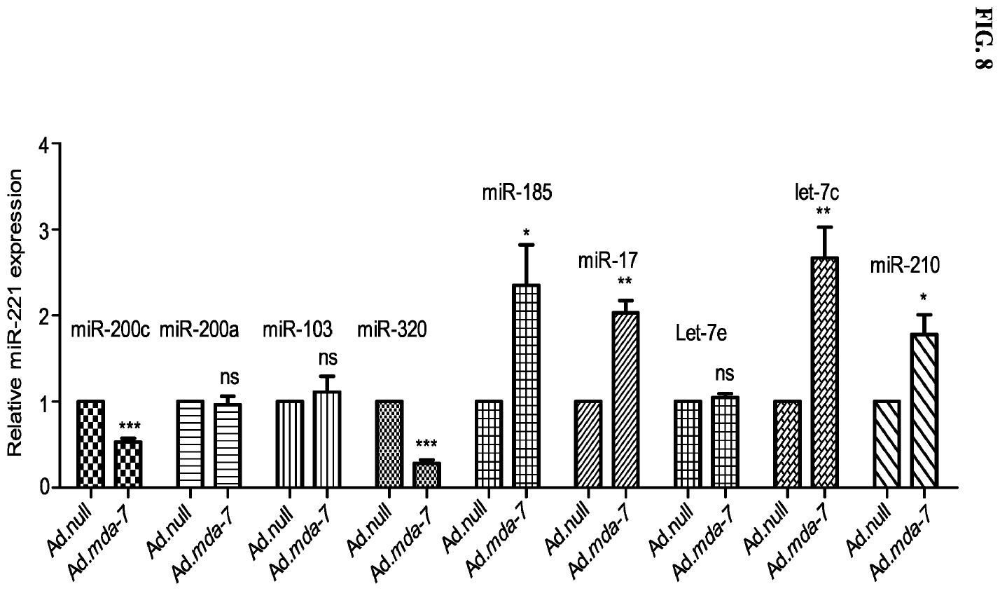

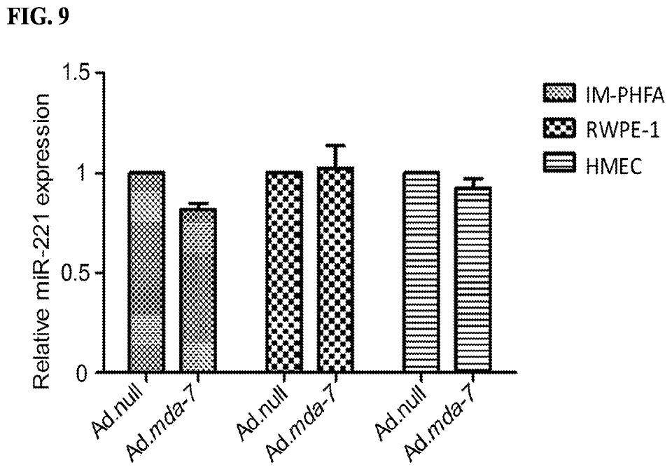

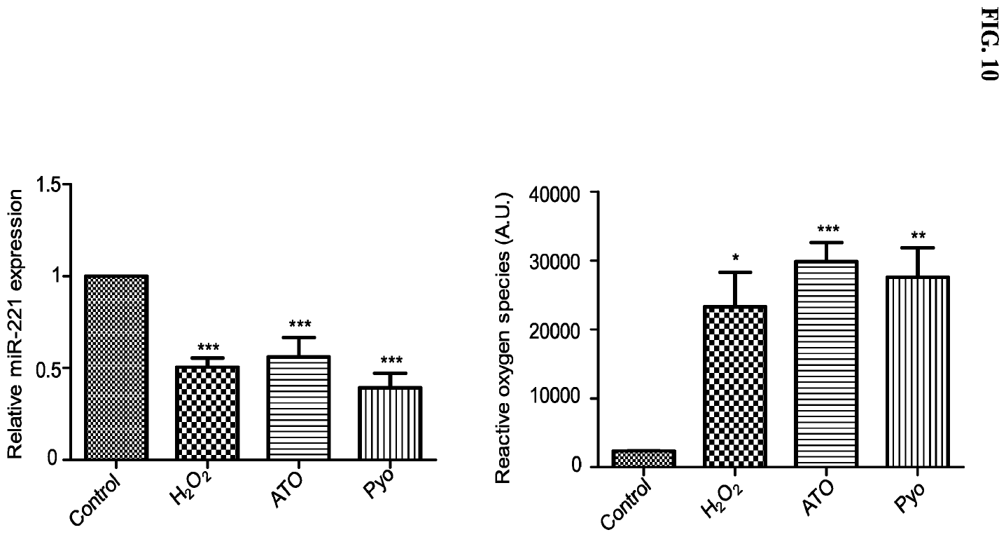

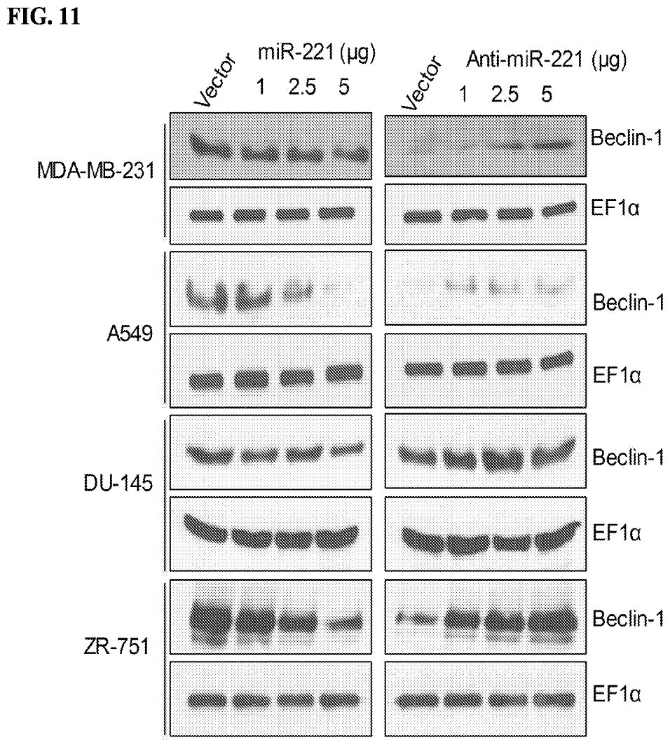

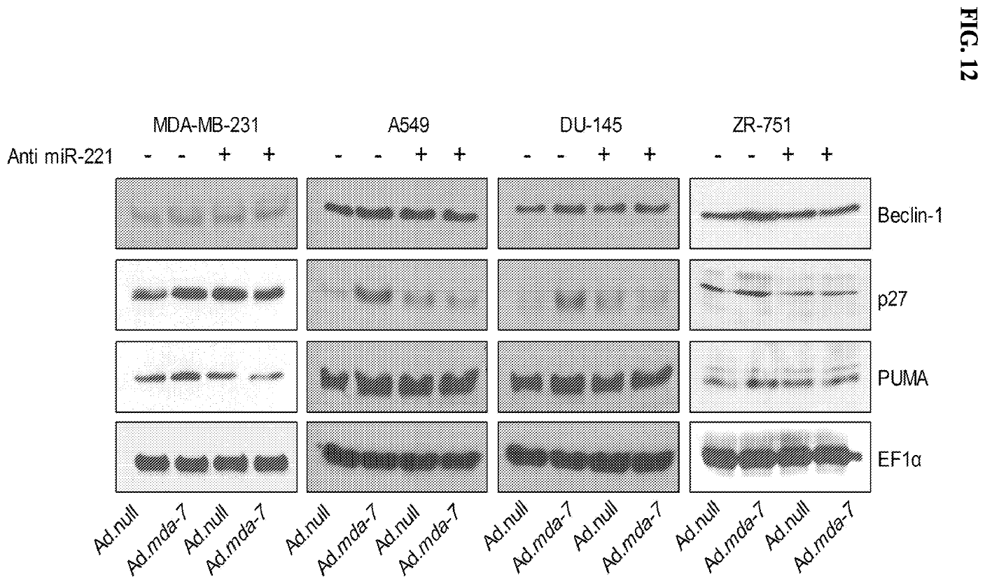

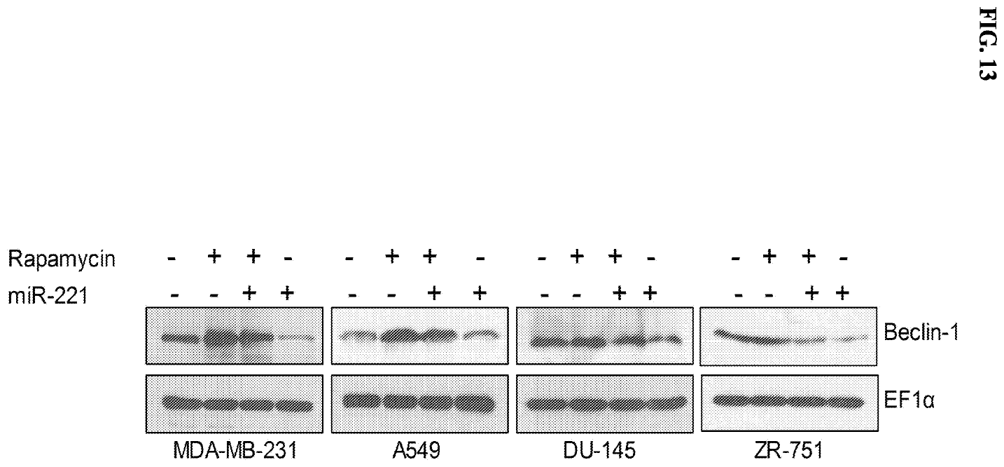

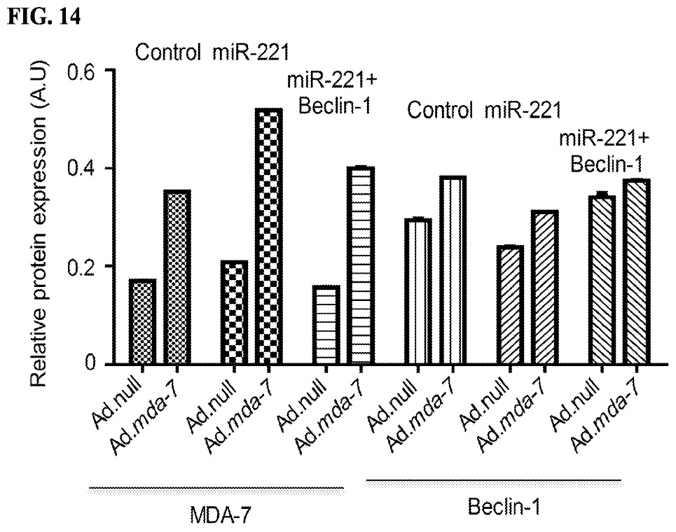

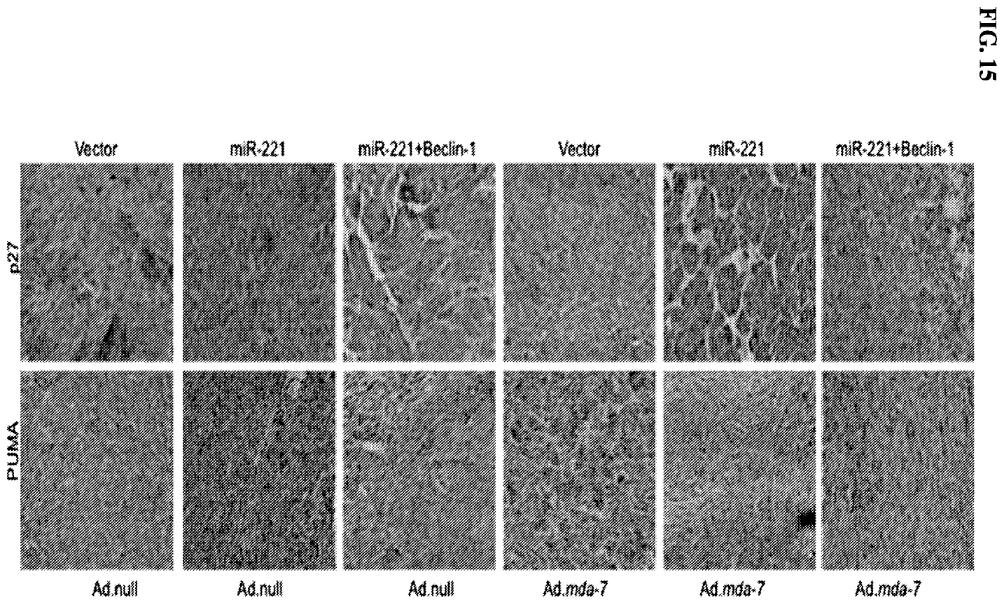

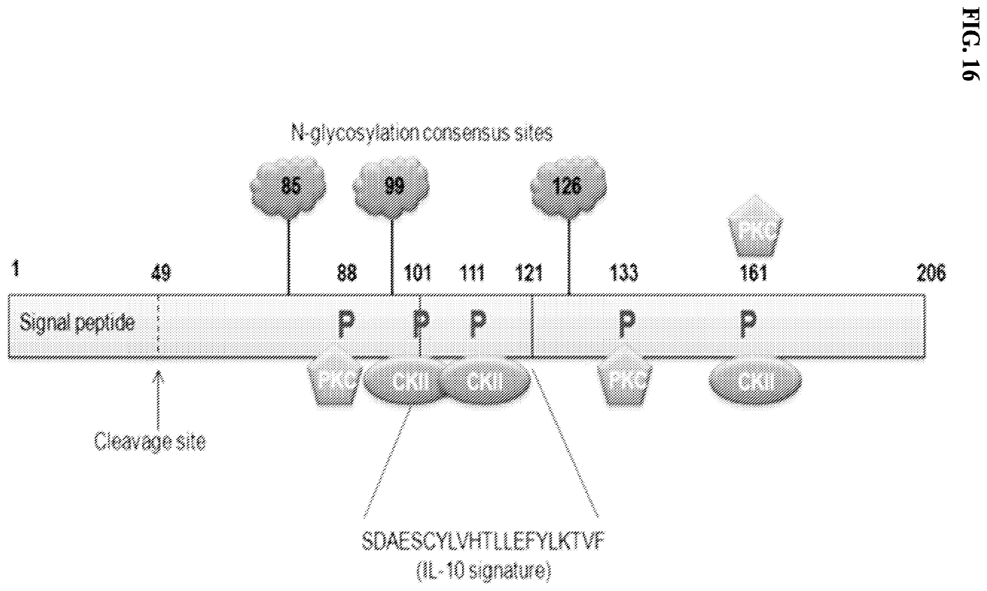

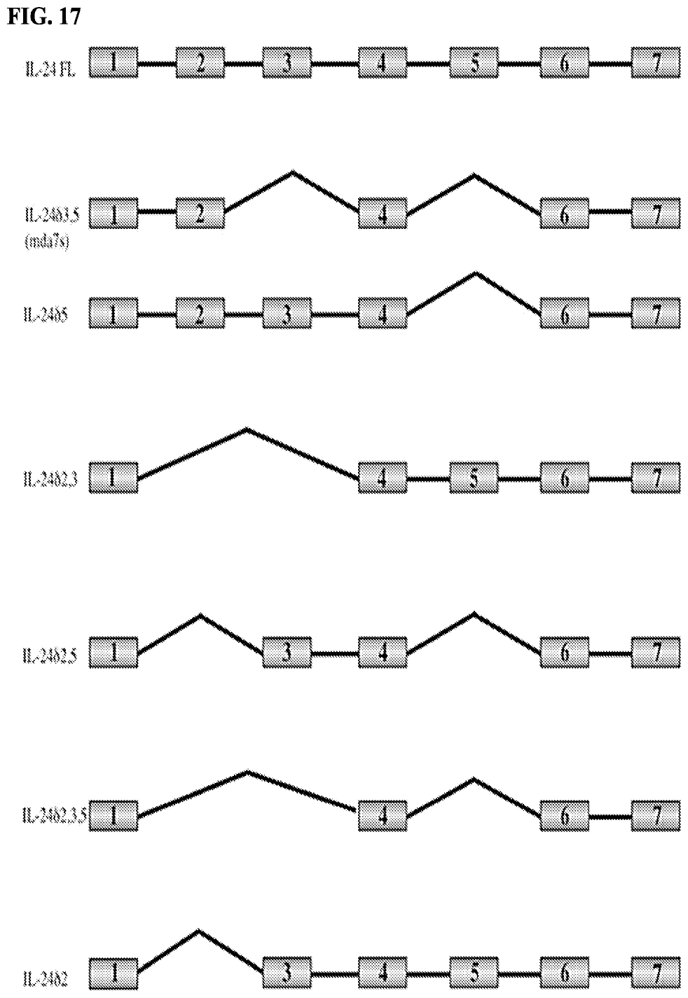

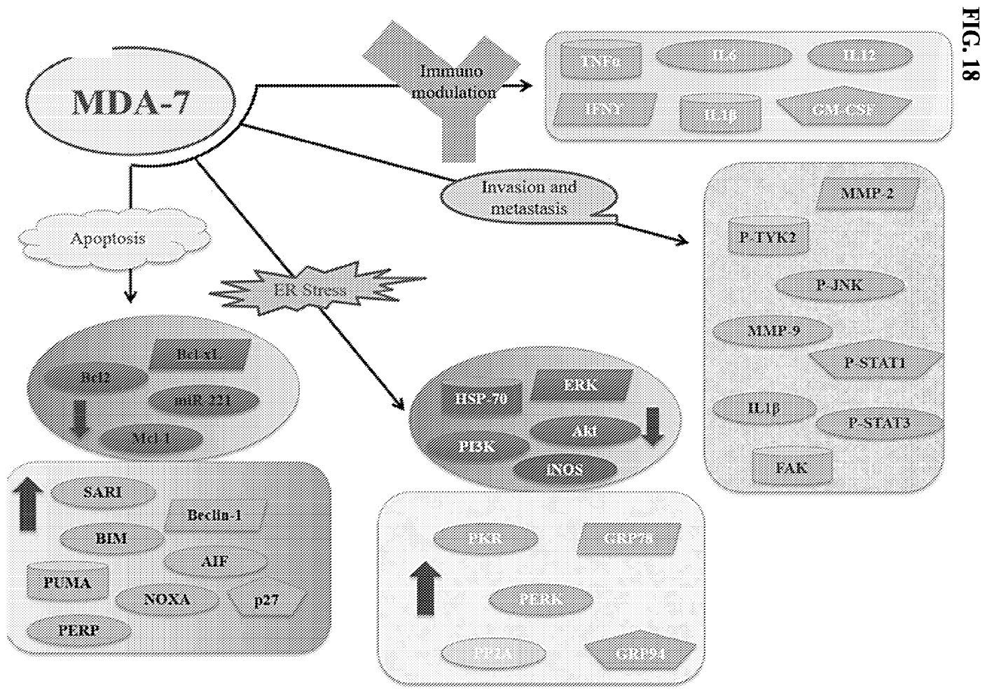

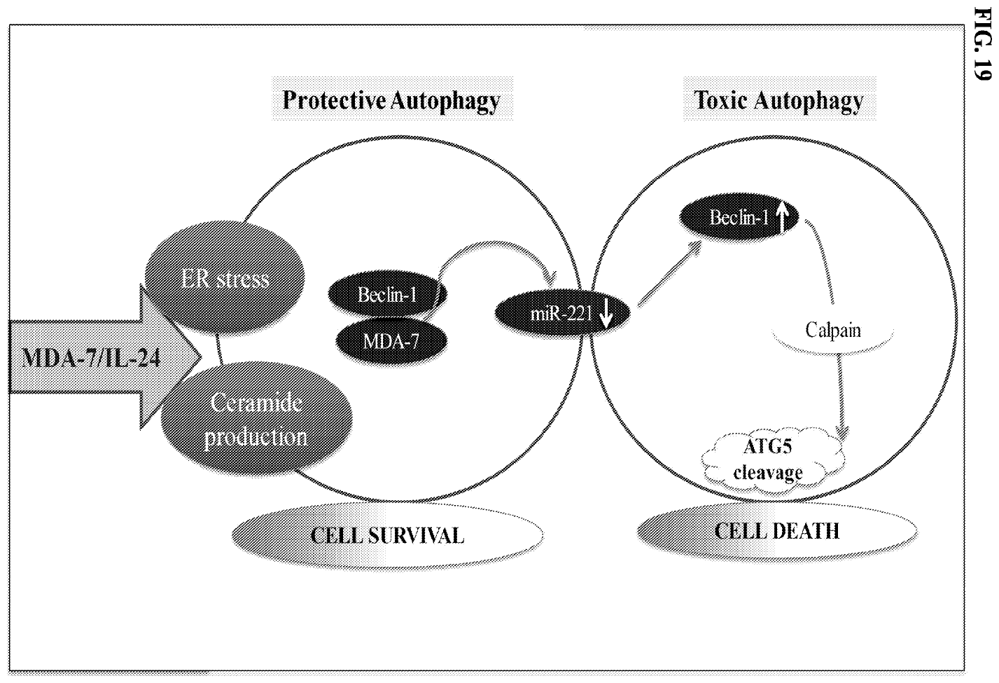

MTT assays were done to check the effect of miR-221 overexpression on mda-7/IL-24-mediated cell growth inhibition. FIG. 3C. Cells were treated as in FIG. 3A and stained with live dead staining kit 72 hours after treatment. Images were obtained using confocal microscopy. FIG. 3D. MDA-MB-231 cells were stably transfected with pCDNA3.1 (vector) or miR-221. After selection, clones were checked for miR-221 expression. FIG. 3E. MDA-MB-231 cells stably overexpressing either pCDNA3.1 (vector) or miR-221 were treated with Ad.null or Ad.mda-7. Two thousand cells were plated and after two weeks they were stained with crystal violet. Number of colonies was counted and the data were plotted in the graph.

FIGS. 4A-4C. mda-7/IL-24 regulates miR-221 expression in a ROS-dependent manner FIG. 4A. MDA-MB-231 cells were infected with Ad.null or Ad.mda-7 (500 vp/cell) for 72 hours, N-acetyl cysteine pretreatment was for 12 hours. Arsenic trioxide (ATO) was added to cells for 12 hours as indicated. RQ-PCR was performed to check the level of miR-221. The level of ROS was measured and presented below. FIG. 4B. MDA-MB-231 cells were treated as above in FIG. 4A. Cells were exposed to Pyocyanin for 12 hours as indicated. RQ-PCR was performed to check the level of miR-221. The level of ROS was measured and is represented below. FIG. 4C. MDA-MB-231 cells were treated as above in FIG. 4A. Hydrogen peroxide was added to cells for 4 hours as indicated. RQ-PCR was performed to determine the level of miR-221 expression. The graphs shown below represent the amount of ROS produced.

FIGS. 5A-5F. Beclin-1 is a direct target of miR-221. FIG. 5A. MDA-MB-231 cells were transfected with control pCDNA3.1 (vector) or miR-221. Western blotting analysis was performed to show the expression of Beclin-1/LC3B/EF1α. FIG. 5B. MDA-MB-231 cells were transfected with increasing concentrations of miR-221, RNA was isolated 48-hours post-transfection and real time PCR was done to check the level of Beclin-1. FIG. 5C. Cells were transfected with a miR-221 construct and then infected with either Ad.null or Ad.mda-7 virus (2000 vp/cell) for 72 hours. Cell lysates were probed with Beclin-1, p27, and PUMA antibodies. EF1α was used as a loading control. FIG. 5D. Reporter gene assays were done in HeLa cells using the 3′UTR Beclin-1 construct; miR-221 over expression significantly decreased the luciferase activity of the wt Beclin-1 UTR. FIG. 5E. MDA-MB-231 cells were transfected with increasing concentrations of anti-miR-221, RNA was isolated after 48 hours and real time PCR was done to check the level of Beclin-1. FIG. 5F. Cells were transfected/treated with the indicated constructs and after 24 hours of transfection they were serum-starved by growth in serum-free medium for 24 hours. Cells were stained with acridine orange and then analyzed by flow cytometry.

FIGS. 6A-6D: Intratumoral injections of mda-7/IL-24 induces miR-221-mediated cell death. FIG. 6A. MDA-MB-231 human breast cancer cells, stably expressing a control pCDNA3.1 vector, miR-221 or miR-221 and Beclin-1, were subcutaneously implanted in both flanks of nude mice. Left sided tumors were treated with 8 intratumoral injections of Ad.mda-7. Ad.null was used as control. A total of 5 mice were studied in each group. Once the control tumors reached maximum allowable limit, tumors were isolated from both flanks. A. Tumor volumes on both flanks were measured and results are presented in a graphical manner FIG. 6B. Graphical representation of the weight of the tumors on both flanks. FIG. 6C. RNA was isolated from the tumor sections (injected tumors) and real time PCR was done to validate the level of miR-221. FIG. 6D. Immunohistochemical analysis of MDA-7/IL-24 and Beclin-1 in tumor sections (injected tumors).

FIG. 7. Schematic representation of Ad. mda-7-induced cell death in cancer cells. MDA-7/IL-24 down regulates miR-221 which in turn up regulates Beclin-1 to induce toxic autophagy and cell death in cancer cells. Additionally, the pathways that are regulated by mda-7/IL-24 are depicted here schematically.

FIG. 8. MDA-MB-231 cells were infected with either Ad.null or Ad.mda-7. miRNA fractions were isolated 72 hours after infection and real time PCR was done using different taqman probes. RNU44 was used as endogenous control.

FIG. 9. Cells were infected with either Ad.null or Ad.mda-7. miRNA fractions were isolated 72 hours after infection and real time PCR was done using the miR-221 taqman probe. RNU44 was used as endogenous control.

FIG. 10. Cells were treated with hydrogen peroxide (100 μM for 4 hours), Arsenic trioxide (10 μM for 12 hours) or pyocyanin (100 μM for 12 hours). RQ-PCR was performed to determine the level of miR-221 expression. The graph on right panel represents the amounts of ROS produced.

FIG. 11. Cells were transfected with increasing concentrations of miR-221 or anti-miR-221. Cell lysates were probed against Beclin-1. EF1α was used as an endogenous control.

FIG. 12. Cells were transfected with an anti-miR-221 construct and then infected with either Ad.null or Ad.mda-7 virus (2000 vp/cell) for 72 hours. Cell lysates were probed with Beclin-1, p27, and PUMA antibodies. EF1α was used as an endogenous control.

FIG. 13. Cells were transfected with miR-221 construct and then treated with Rapamycin. Cell lysates were probed with beclin-1 antibody. EF1α was used as an endogenous control.

FIG. 14. Quantification of MDA-7/IL-24 and Beclin-1 in immunohistochemistry images (FIG. 6D). This data is graphically represented.

FIG. 15. Immunohistochemical analysis of p27 and PUMA in tumor sections.

FIG. 16. Schematic representation of MDA-7/IL-24 protein with predicted and established domains and protein modification sites indicated. Cleavage of the 49-amino acid signal peptide allows for secretion of the MDA-7/IL-24 protein. The IL-10 signature sequence is located between amino acid 101 and 121. N-glycosylation can occur at amino acids 85, 99 and 126. Protein kinase C consensus phosphorylation sites are present at amino acids 88, 133 and 161. Casein kinase II (CMI) consensus phosphorylation sites are present at amino acids 101, 111 and 161. Numbers indicate amino acids. Not drawn to scale. (Figure reproduced from Menezes et al., 2014).

FIG. 17. Schematic representation of the splice isoforms of MDA-7/IL-24. (Figure reproduced from Whitaker et al., 2011).

FIG. 18. Schematic representation of the pathways regulated by MDA-7/IL-24. MDA-7/IL-24 regulates both pro and anti-apoptotic molecules to induce tumor specific cell death. This involves a series of signaling events including down regulation of Mcl-1 and Bcl-xL and activation of tumor suppressors i.e. SARI, PUMA, AIF, PERP and others as shown in the figure. Also the cytokine induces ER stress and regulates a number of genes/proteins to block invasion and metastasis. MDA-7/IL-24 also modulates the immune pathways by deregulating a number of cytokines, which in turn activates the immune system to induce cytotoxic cell death.

FIG. 19. Model depicting the molecular mechanism of MDA-7/IL-24-mediated autophagy induction. MDA-7/IL-24 regulates autophagy mediated through ER stress and ceramide production. Also MDA-7/IL-24 down regulates miR-221, which in turn upregulates Beclin-1 to induce toxic autophagy leading to cell death. The transition of protective to toxic autophagy is mediated by the cleavage of ATGS by Calpain.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

I. Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

The terms “microRNA” and “miRNA,” and abbreviation “miR” refer to small non-coding RNA molecules (e.g., 20 nucleotides in length) that play a role in RNA (e.g., mRNA) silencing and post-transcriptional regulation of gene expression. miRNAs accomplish these functions via base pairing with complementary sequences within RNA molecules (e.g., mRNA) resulting in cleavage of the bound RNA (e.g., mRNA), destabilization of the mRNA (e.g., mRNA) through shortening of the poly(A) tail, and/or decreasing translation efficiency of the RNA (e.g., mRNA) by ribosomes. A “microRNA” or “miRNA,” is a single-stranded nucleic acid forming part of or derived from a double-stranded nucleic acid which includes complementary portions of substantial or complete identity also referred to as doublestranded hairpin structures. Upon intracellular processing of the hairpin structure the miRNA is released and able to bind its cellular target sequence which it completely or partially complementary to. A miRNA has the ability to reduce or inhibit expression of a gene or target gene when expressed in the same cell as the gene or target gene. In one embodiment, a miRNA refers to a nucleic acid that has substantial or complete identity to a target sequence. In embodiments, the miRNA inhibits gene expression by interacting with a complementary cellular mRNA thereby interfering with the expression of the complementary mRNA. Typically, the miRNA is at least about 15-50 nucleotides in length. In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

The terms “microRNA-221,” “miR-221,” and “mir-221” refer to a microRNA (including homologs, isoforms, and functional fragments thereof) with miR-221 activity. “miR-221 activity” as referred to herein is the ability of a miRNA to bind cellular sequences complementary to miR-221. In embodiments, the miR-221 is substantially identical to the mircoRNA identified by HGNC:31601 or a variant, homolog, or isoform having substantial identity thereto. In embodiments, miR-221 is substantially identical to the nucleic acid sequence set forth in RefSeq (mRNA) NR_029635, or a variant, homolog, or isoform having substantial identity thereto. In embodiments, the nucleic acid sequence is the sequence known at the time of filing of the present application. In embodiments, the miR-221 has a sequence identity of at least 70%, 75%, 80%, 85%, 90%, 95%, 96%, 97%, 98%, or 99% over the whole sequence or a portion of the sequence identified by HGNC:31601 or RefSeq (mRNA) NR_029635, a variant, homolog, or isoform having substantial identity thereto. The term includes any recombinant or naturally-occurring form of miR-221 or variants, homologs, isoforms thereof that maintain miR-221 activity (e.g. within at least 30%, 40%, 50%, 60%, 70%, 80%, 90%, 95%, or 100% activity compared to wildtype miR-221). In some aspects, the variants, homologs, or isoforms have at least 90%, 95%, 96%, 97%, 98%, 99% or 100% nucleic acid sequence identity across the whole sequence or a portion of the sequence (e.g., a 50, 100, 150 or 200 continuous amino acid portion) compared to a naturally occurring miR-221 microRNA.

The term “probe” or “primer”, as used herein, is defined to be one or more nucleic acid fragments whose specific hybridization to a sample can be detected. A probe or primer can be of any length depending on the particular technique it will be used for. For example, PCR primers (e.g., real time PCR) are generally between 10 and 40 nucleotides in length, while nucleic acid probes for, e.g., a Southern blot, can be more than a hundred nucleotides in length. The probe may be unlabeled or labeled (e.g., with a deteactable moiety) as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The words “complementary” or “complementarity” refer to the ability of a nucleic acid in a polynucleotide to form a base pair (e.g., hybridize) with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing. In embodiments, a nucleic aicd that is complementary to a target nucleic acid is capable of hybridizing to the target nucleic acid under stringent hybridation conditions.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected protein corresponds to, for example, serine at position 101 of a human MDA-7 protein when the selected residue occupies the same essential spatial or other structural relationship as a serine at position 101 in human MDA-7 protein. In some embodiments, where a selected protein is aligned for maximum homology with the human MDA-7 protein, the position in the aligned selected protein aligning with serine 101 is said to correspond to serine 101. Instead of a primary sequence alignment, a three dimensional structural alignment can also be used, e.g., where the structure of the selected protein is aligned for maximum correspondence with the human MDA-101 protein and the overall structures compared. In this case, an amino acid that occupies the same essential position as serine 101 in the structural model is said to correspond to the serine 101 residue.

“Conservatively modified variants” applies to both amino acid and nucleic acid sequences. With respect to particular nucleic acid sequences, “conservatively modified variants” refers to those nucleic acids that encode identical or essentially identical amino acid sequences.

Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “MDA-7,” “IL-24,” or “MDA-7/IL-24” refer to a protein (including homologs, isoforms, and functional fragments thereof) with MDA-7 activity. The term includes any recombinant or naturally-occurring form of MDA-7 or variants, homologs, or isoforms thereof that maintain MDA-7 activity (e.g. within at least 30%, 40%, 50%, 60%, 70%, 80%, 90%, 95%, or 100% activity compared to wildtype MDA-7). In some aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring MDA-7 protein. In embodiments, the MDA-7 protein is substantially identical to the protein identified by Accession No. NP_006841 or a variant or homolog having substantial identity thereto. In embodiments, the MDA-7 protein is substantially identical to the protein identified by UniProt Q13007 or a variant or homolog having substantial identity thereto. In embodiments, the IL-24 gene is substantially identical to the nucleic acid sequence set forth in RefSeq (mRNA) NM_006850, or a variant or homolog having substantial identity thereto. In embodiments, the IL-24 gene is substantially identical to the nucleic acid sequence set forth in Ensembl reference number ENSG00000162892, or a variant or homolog having substantial identity thereto. In embodiments, the amino acid sequence or nucleic acid sequence is the sequence known at the time of filing of the present application.

The term “beclin-1” refers to a protein (including homologs, isoforms, and functional fragments thereof) with beclin-1 activity. The term includes any recombinant or naturally-occurring form of beclin-1 or variants, homologs, or isoforms thereof that maintain beclin-1 activity (e.g. within at least 30%, 40%, 50%, 60%, 70%, 80%, 90%, 95%, or 100% activity compared to wildtype beclin-1). In some aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring beclin-1 protein. In embodiments, beclin-1 is substantially identical to the protein identified by Accession No. NP_003757 or a variant or homolog having substantial identity thereto. In embodiments, beclin-1 is substantially identical to the protein identified by UniProt Q14457, or variants, homologs, or isoforms having substantial identity thereto. In embodiments, the gene encoding beclin-1 is substantially identical to the nucleic acid sequence set forth in RefSeq (mRNA) NM_003766, or variants, homologs, or isoforms having substantial identity thereto. In embodiments, the gene encoding beclin-1 is substantially identical to the nucleic acid sequence set forth in Ensembl reference number ENSG00000126581, or variants, homologs, or isoforms having substantial identity thereto. In embodiments, the amino acid sequence or nucleic acid sequence is the sequence known at the time of filing of the present application.

The terms “TIMP3” and “metalloproteainase inhibitor 3” refer to a protein (including homologs, isoforms, and functional fragments thereof) with TIMP3 activity. The term includes any recombinant or naturally-occurring form of TIMP3 or variants, homologs, or isoforms thereof that maintain TIMP3 activity (e.g. within at least 30%, 40%, 50%, 60%, 70%, 80%, 90%, 95%, or 100% activity compared to wildtype TIMP3). In some aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring TIMP3 protein. In embodiments, the TIMP3 protein is substantially identical to the protein identified by Accession No. NP_000353 or variants, homologs, or isoforms having substantial identity thereto. In embodiments, the TIMP3 protein is substantially identical to the protein identified by UniProt P35625, or variants, homologs, or isoforms having substantial identity thereto. In embodiments, the gene encoding TIMP3 is substantially identical to the nucleic acid sequence set forth in RefSeq (mRNA) NM_000362, or variants, homologs, or isoforms having substantial identity thereto. In embodiments, the gene encoding TIMP3 is substantially identical to the nucleic acid sequence set forth in Ensembl reference number ENSG00000100234, or variants, homologs, or isoforms having substantial identity thereto. In embodiments, the amino acid sequence or nucleic acid sequence is the sequence known at the time of filing of the present application.

The terms “BMP2” and “bone morphogenetic protein 2” refer to a protein (including homologs, isoforms, and functional fragments thereof) with BMP2 activity. The term includes any recombinant or naturally-occurring form of BMP2 or variants, homologs, or isoforms thereof that maintain BMP2 activity (e.g. within at least 30%, 40%, 50%, 60%, 70%, 80%, 90%, 95%, or 100% activity compared to wildtype BMP2). In some aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring BMP2 protein. In embodiments, the BMP2 protein is substantially identical to the protein identified by Accession No. NP_001191 or variants, homologs, or isoforms having substantial identity thereto. In embodiments, the BMP2 protein is substantially identical to the protein identified by UniProt P12643 or variants, homologs, or isoforms having substantial identity thereto. In embodiments, the gene encoding BMP2 is substantially identical to the nucleic acid sequence set forth in RefSeq (mRNA) NM_001200, or a variant or homolog having substantial identity thereto. In embodiments, the gnene encoding BMP2 is substantially identical to the nucleic acid sequence set forth in Ensembl reference number ENSG00000125845, or variants, homologs, or isoforms having substantial identity thereto. In embodiments, the amino acid sequence or nucleic acid sequence is the sequence known at the time of filing of the present application.

The terms “secreted uPAR isoform2” or “uPAR2” refer to a protein (including homologs, isoforms, and functional fragments thereof) with secreted uPAR isoform2 activity. The term includes any recombinant or naturally-occurring form of secreted uPAR isoform2 or variants, homologs, or isoforms thereof that maintain secreted uPAR isoform2 activity (e.g. within at least 30%, 40%, 50%, 60%, 70%, 80%, 90%, 95%, or 100% activity compared to wildtype secreted uPAR isoform2). In some aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring secreted uPAR isoform2 protein. In embodiments, the secreted uPAR isoform2 protein is substantially identical to the protein identified by Accession No. NP_001005376 or variants, homologs, or isoforms having substantial identity thereto. In embodiments, the secreted uPAR isoform2 protein is substantially identical to the protein identified by UniProt Q03405-2 or variants, homologs, or isoforms having substantial identity thereto. In embodiments, the gene which encodes uPAR2, is substantially identical to the nucleic acid sequence set forth in RefSeq (mRNA) NM_001005376, or variants, homologs, or isoforms having substantial identity thereto. In embodiments, the gene that encodes uPAR2 is substantially identical to the nucleic acid sequence set forth in Ensembl reference number ENSG00000011422, or variants, homologs, or isoforms having substantial identity thereto. In embodiments, the amino acid sequence or nucleic acid sequence is the sequence known at the time of filing of the present application.

The terms “MMP” or “matrix metalloproteinase” refer to a family of calcium-dependent zinc-containing endopeptidases. The family includes MMP1, MMP2, MMP3, MMPI, MMP8, MMP9, MMP10, MMP11, MMP12, MMP13, MMP14, MMP15, MMP16, MMP17, MMP19, MMP20, MMP21, MMP23, MMP23B, MMP24, MMP25, MMP26, MMP27, and MMP28. The term includes any recombinant or naturally-occurring form of MMP or variants, homologs, or isoforms thereof that maintain MMP activity (e.g. within at least 30%, 40%, 50%, 60%, 70%, 80%, 90%, 95%, or 100% activity compared to wildtype MMP). In some aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring MMP protein.

In embodiments, the MMP protein is substantially identical to the protein identified by Accession No. NP_002412 or variants, homologs, or isoforms having substantial identity thereto. In embodiments, the MMP protein is substantially identical to the protein identified by UniProt P03956 or variants, homologs, or isoforms having substantial identity thereto. In embodiments, the gene encoding MMP is substantially identical to the nucleic acid sequence set forth in RefSeq (mRNA) Accession No. NM_002421, or variants, homologs, or isoforms having substantial identity thereto. In embodiments, the gene encoding MMP is substantially identical to the nucleic acid sequence set forth in Ensembl reference number ENSG00000196611, or variants, homologs, or isoforms having substantial identity thereto.

In embodiments, the MMP protein is substantially identical to the protein identified by Accession No. NP_004521 or variants, homologs, or isoforms having substantial identity thereto. In embodiments, the MMP protein is substantially identical to the protein identified by UniProt P08253 or variants, homologs, or isoforms having substantial identity thereto. In embodiments, the gene encoding MMP is substantially identical to the nucleic acid sequence set forth in RefSeq (mRNA) Accession No. NM_004530, or variants, homologs, or isoforms having substantial identity thereto. In embodiments, the gene encoding MMP is substantially identical to the nucleic acid sequence set forth in Ensembl reference number ENSG00000087245, or variants, homologs, or isoforms having substantial identity thereto.

In embodiments, the MMP protein is substantially identical to the protein identified by Accession No. NP_002413 or variants, homologs, or isoforms having substantial identity thereto. In embodiments, the MMP protein is substantially identical to the protein identified by UniProt P08254 or variants, homologs, or isoforms having substantial identity thereto. In embodiments, the gene encoding MMP is substantially identical to the nucleic acid sequence set forth in RefSeq (mRNA) Accession No. NM_002422, or variants, homologs, or isoforms having substantial identity thereto. In embodiments, the gene encoding MMP is substantially identical to the nucleic acid sequence set forth in Ensembl reference number ENSG00000149968 or variants, homologs, or isoforms having substantial identity thereto.

In embodiments, the MMP protein is substantially identical to the protein identified by

Accession No. NP_002414 or variants, homologs, or isoforms having substantial identity thereto. In embodiments, the MMP protein is substantially identical to the protein identified by UniProt P09237 or variants, homologs, or isoforms having substantial identity thereto. In embodiments, the gene encoding MMP is substantially identical to the nucleic acid sequence set forth in RefSeq (mRNA) Accession No. NM_002423, or variants, homologs, or isoforms having substantial identity thereto. In embodiments, the gene encoding MMP is substantially identical to the nucleic acid sequence set forth in Ensembl reference number ENSG00000137673 or variants, homologs, or isoforms having substantial identity thereto.

In embodiments, the MMP protein is substantially identical to the protein identified by Accession No. NP_002415 or variants, homologs, or isoforms having substantial identity thereto. In embodiments, the MMP protein is substantially identical to the protein identified by UniProt P22894 or variants, homologs, or isoforms having substantial identity thereto. In embodiments, the gene encoding MMP, is substantially identical to the nucleic acid sequence set forth in RefSeq (mRNA) Accession No. NM_002424 or variants, homologs, or isoforms having substantial identity thereto. In embodiments, the gene encoding MMP is substantially identical to the nucleic acid sequence set forth in Ensembl reference number ENSG00000118113, or variants, homologs, or isoforms having substantial identity thereto.

In embodiments, the MMP protein is substantially identical to the protein identified by Accession No. NP_004985 or variants, homologs, or isoforms having substantial identity thereto. In embodiments, the MMP protein is substantially identical to the protein identified by UniProt P14780 or variants, homologs, or isoforms having substantial identity thereto. In embodiments, the gene encoding MMP is substantially identical to the nucleic acid sequence set forth in RefSeq (mRNA) Accession No. NM_004994, or variants, homologs, or isoforms having substantial identity thereto. In embodiments, the gene encoding MMP is substantially identical to the nucleic acid sequence set forth in Ensembl reference number ENSG00000100985, or variants, homologs, or isoforms having substantial identity thereto.

In embodiments, the MMP protein is substantially identical to the protein identified by Accession No. NP_002416 or variants, homologs, or isoforms having substantial identity thereto. In embodiments, the MMP protein is substantially identical to the protein identified by UniProt P09238 or variants, homologs, or isoforms having substantial identity thereto. In embodiments, the gene encoding MMP is substantially identical to the nucleic acid sequence set forth in RefSeq (mRNA) Accession No. NM_002425, or variants, homologs, or isoforms having substantial identity thereto. In embodiments, the gene encoding MMP is substantially identical to the nucleic acid sequence set forth in Ensembl reference number ENSG00000166670, or variants, homologs, or isoforms having substantial identity thereto.

In embodiments, the MMP protein is substantially identical to the protein identified by Accession No. NP_005931 or variants, homologs, or isoforms having substantial identity thereto. In embodiments, the MMP protein is substantially identical to the protein identified by UniProt P24347 or variants, homologs, or isoforms having substantial identity thereto. In embodiments, the gene encoding MMP is substantially identical to the nucleic acid sequence set forth in RefSeq (mRNA) Accession No. NM_005940, or variants, homologs, or isoforms having substantial identity thereto. In embodiments, the gene encoding MMP is substantially identical to the nucleic acid sequence set forth in Ensembl reference number ENSG00000275365, or variants, homologs, or isoforms having substantial identity thereto.

In embodiments, the MMP protein is substantially identical to the protein identified by Accession No. NP_002417 or variants, homologs, or isoforms having substantial identity thereto. In embodiments, the MMP protein is substantially identical to the protein identified by UniProt P39900 or variants, homologs, or isoforms having substantial identity thereto. In embodiments, the gene encoding MMP is substantially identical to the nucleic acid sequence set forth in RefSeq (mRNA) Accession No. NM_002426, or variants, homologs, or isoforms having substantial identity thereto. In embodiments, the gene encoding MMP is substantially identical to the nucleic acid sequence set forth in Ensembl reference number ENSG00000262406, or variants, homologs, or isoforms having substantial identity thereto.

In embodiments, the MMP protein is substantially identical to the protein identified by Accession No. NP_002418 or variants, homologs, or isoforms having substantial identity thereto. In embodiments, the MMP protein is substantially identical to the protein identified by UniProt P45452 or variants, homologs, or isoforms having substantial identity thereto. In embodiments, the gene encoding MMP is substantially identical to the nucleic acid sequence set forth in RefSeq (mRNA) Accession No. NM_002427, or variants, homologs, or isoforms having substantial identity thereto. In embodiments, the gene encoding MMP is substantially identical to the nucleic acid sequence set forth in Ensembl reference number ENSG00000137745, or variants, homologs, or isoforms having substantial identity thereto.

In embodiments, the MMP protein is substantially identical to the protein identified by Accession No. NP_004986 or variants, homologs, or isoforms having substantial identity thereto. In embodiments, the MMP protein is substantially identical to the protein identified by UniProt P50281 or variants, homologs, or isoforms having substantial identity thereto. In embodiments, the gene encoding MMP is substantially identical to the nucleic acid sequence set forth in RefSeq (mRNA) Accession No. NM_004995, or variants, homologs, or isoforms having substantial identity thereto. In embodiments, the gene encoding MMP is substantially identical to the nucleic acid sequence set forth in Ensembl reference number ENSG00000157227 , or variants, homologs, or isoforms having substantial identity thereto.

In embodiments, the MMP protein is substantially identical to the protein identified by Accession No. NP_002419 or variants, homologs, or isoforms having substantial identity thereto. In embodiments, the MMP protein is substantially identical to the protein identified by UniProt P51511 or variants, homologs, or isoforms having substantial identity thereto. In embodiments, the gene encoding MMP is substantially identical to the nucleic acid sequence set forth in RefSeq (mRNA) Accession No. NM_002428, or variants, homologs, or isoforms having substantial identity thereto. In embodiments, the gene encoding MMP is substantially identical to the nucleic acid sequence set forth in Ensembl reference number ENSG00000102996, or variants, homologs, or isoforms having substantial identity thereto.

In embodiments, the MMP protein is substantially identical to the protein identified by Accession No. NP_005932 or variants, homologs, or isoforms having substantial identity thereto. In embodiments, the MMP protein is substantially identical to the protein identified by UniProt P51512 or variants, homologs, or isoforms having substantial identity thereto. In embodiments, the gene encoding MMP is substantially identical to the nucleic acid sequence set forth in RefSeq (mRNA) Accession No. NM_005941, or variants, homologs, or isoforms having substantial identity thereto. In embodiments, the gene encoding MMP is substantially identical to the nucleic acid sequence set forth in Ensembl reference number ENSG00000156103, or variants, homologs, or isoforms having substantial identity thereto.

In embodiments, the MMP protein is substantially identical to the protein identified by Accession No. NP_057239 or variants, homologs, or isoforms having substantial identity thereto. In embodiments, the MMP protein is substantially identical to the protein identified by UniProt Q9ULZ9 or variants, homologs, or isoforms having substantial identity thereto. In embodiments, the gene encoding MMP is substantially identical to the nucleic acid sequence set forth in RefSeq (mRNA) Accession No. NM_016155, or variants, homologs, or isoforms having substantial identity thereto. In embodiments, the gene encoding MMP is substantially identical to the nucleic acid sequence set forth in Ensembl reference number ENSG00000198598, or variants, homologs, or isoforms having substantial identity thereto.

In embodiments, the MMP protein is substantially identical to the protein identified by Accession No. NP_002420 or variants, homologs, or isoforms having substantial identity thereto. In embodiments, the MMP protein is substantially identical to the protein identified by UniProt Q99542 or variants, homologs, or isoforms having substantial identity thereto. In embodiments, the gene encoding MMP is substantially identical to the nucleic acid sequence set forth in RefSeq (mRNA) Accession No. NM_002429, or variants, homologs, or isoforms having substantial identity thereto. In embodiments, the gene encoding MMP is substantially identical to the nucleic acid sequence set forth in Ensembl reference number ENSG00000123342, or variants, homologs, or isoforms having substantial identity thereto.

In embodiments, the MMP protein is substantially identical to the protein identified by Accession No. NP_004762 or variants, homologs, or isoforms having substantial identity thereto. In embodiments, the MMP protein is substantially identical to the protein identified by UniProt O60882 or variants, homologs, or isoforms having substantial identity thereto. In embodiments, the gene encoding MMP is substantially identical to the nucleic acid sequence set forth in RefSeq (mRNA) Accession No. NM_004771, or variants, homologs, or isoforms having substantial identity thereto. In embodiments, the gene encoding MMP is substantially identical to the nucleic acid sequence set forth in Ensembl reference number ENSG00000137674, or variants, homologs, or isoforms having substantial identity thereto.

In embodiments, the MMP protein is substantially identical to the protein identified by Accession No. NP_671724 or variants, homologs, or isoforms having substantial identity thereto. In embodiments, the MMP protein is substantially identical to the protein identified by UniProt Q8N119 or variants, homologs, or isoforms having substantial identity thereto. In embodiments, the gene encoding MMP is substantially identical to the nucleic acid sequence set forth in RefSeq (mRNA) Accession No. NM_147191, or variants, homologs, or isoforms having substantial identity thereto. In embodiments, the gene encoding MMP is substantially identical to the nucleic acid sequence set forth in Ensembl reference number ENSG00000154485, or variants, homologs, or isoforms having substantial identity thereto.

In embodiments, the MMP protein is substantially identical to the protein identified by Accession No. NP_008914 or variants, homologs, or isoforms having substantial identity thereto. In embodiments, the MMP protein is substantially identical to the protein identified by UniProt 075900 or variants, homologs, or isoforms having substantial identity thereto. In embodiments, the gene encoding MMP is substantially identical to the nucleic acid sequence set forth in RefSeq (mRNA) Accession No. NM_006983, or variants, homologs, or isoforms having substantial identity thereto. In embodiments, the gene encoding MMP is substantially identical to the nucleic acid sequence set forth in Ensembl reference number ENSG00000189409, or variants, homologs, or isoforms having substantial identity thereto.

In embodiments, the MMP protein is substantially identical to the protein identified by Accession No. NP_006681 or variants, homologs, or isoforms having substantial identity thereto. In embodiments, the MMP protein is substantially identical to the protein identified by UniProt Q9Y5R2 or variants, homologs, or isoforms having substantial identity thereto. In embodiments, the gene encoding MMP is substantially identical to the nucleic acid sequence set forth in RefSeq (mRNA) Accession No. NM_006690, or variants, homologs, or isoforms having substantial identity thereto. In embodiments, the gene encoding MMP is substantially identical to the nucleic acid sequence set forth in Ensembl reference number ENSG00000125966, or variants, homologs, or isoforms having substantial identity thereto.

In embodiments, the MMP protein is substantially identical to the protein identified by Accession No. NP_071913 or variants, homologs, or isoforms having substantial identity thereto. In embodiments, the MMP protein is substantially identical to the protein identified by UniProt Q9NPA2 or variants, homologs, or isoforms having substantial identity thereto. In embodiments, the gene encoding MMP is substantially identical to the nucleic acid sequence set forth in RefSeq (mRNA) Accession No. NM_022468, or variants, homologs, or isoforms having substantial identity thereto. In embodiments, the gene encoding MMP is substantially identical to the nucleic acid sequence set forth in Ensembl reference number ENSG00000008516, or variants, homologs, or isoforms having substantial identity thereto.

In embodiments, the MMP protein is substantially identical to the protein identified by Accession No. NP_068573 or variants, homologs, or isoforms having substantial identity thereto. In embodiments, the MMP protein is substantially identical to the protein identified by UniProt Q9NRE1 or variants, homologs, or isoforms having substantial identity thereto. In embodiments, the gene encoding MMP is substantially identical to the nucleic acid sequence set forth in RefSeq (mRNA) Accession No. NM_021801, or variants, homologs, or isoforms having substantial identity thereto. In embodiments, the gene encoding MMP is substantially identical to the nucleic acid sequence set forth in Ensembl reference number ENSG00000167346, or variants, homologs, or isoforms having substantial identity thereto.

In embodiments, the MMP protein is substantially identical to the protein identified by Accession No. NP_071405 or variants, homologs, or isoforms having substantial identity thereto. In embodiments, the MMP protein is substantially identical to the protein identified by UniProt Q9H306 or variants, homologs, or isoforms having substantial identity thereto. In embodiments, the gene encoding MMP is substantially identical to the nucleic acid sequence set forth in RefSeq (mRNA) Accession No. NM_022122, or variants, homologs, or isoforms having substantial identity thereto. In embodiments, the gene encoding MMP is substantially identical to the nucleic acid sequence set forth in Ensembl reference number ENSG00000137675, or variants, homologs, or isoforms having substantial identity thereto.

In embodiments, the MMP protein is substantially identical to the protein identified by Accession No. NP_077278 or variants, homologs, or isoforms having substantial identity thereto. In embodiments, the MMP protein is substantially identical to the protein identified by UniProt Q9H239 or variants, homologs, or isoforms having substantial identity thereto. In embodiments, the gene encoding MMP is substantially identical to the nucleic acid sequence set forth in RefSeq (mRNA) Accession No. NM_024302, or variants, homologs, or isoforms having substantial identity thereto. In embodiments, the gene encoding MMP is substantially identical to the nucleic acid sequence set forth in Ensembl reference number ENSG00000278843, or variants, homologs, or isoforms having substantial identity thereto.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹ Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, immunoprecipitation, flow cytometry, immunofluorescence, immunohistochemistry, etc.) and/or nucleic acids (e.g., PCR (e.g., real time/quantitative, reverse transcriptase); in situ hybridization, including FISH; Southern blotting; Northern blotting, etc.).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia , Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome,vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo,asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

The term “infection” or “infectious disease” refers to a disease or condition that can be caused by organisms such as a bacterium, virus, fungi or any other pathogenic microbial agents. In embodiments, the infectious diseases is a viral infection (e.g., HIV, SARS, HPV, influenza), or bacterial colonization in the human gastrointestinal tract (e.g., pathenogenic bacterial colonization). In embodiments, the infectious disease is associated with elevated expression of mda-9. In embodiments, the infectious disease is characterized by the presence of virus shedding (e.g., HIV viral shedding or Herpes viral shedding). In embodiments, the infectious disease is a bacterial infection. In embodiments, the infectious disease is a gram-positive bacterial infection. In embodiments, the infectious disease is a Staphylococcus aureus infection. In embodiments, the infectious disease is Gram-positive or Gram-negative bacterial infection. In embodiments, the infectious disease is an infection associated with S. aureus, E. facium, E. faecalis, K. pneumonoiaea, H. influenzaea, or P. aeruginosa. In embodiments, the infectious disease is a S. aureus, E. facium, E. faecalis, K. pneumonoiaea, H. influenzaea, or P. aeruginosa infection.

The term “cardiovascular disease” refers to a disease or condition wherein blood vessels of the cardiovascular system are blocked, narrowed, or calcified, thereby increasing the likelihood of heart attack, heart failure, angina (chest pain), peripheral artery disease, aneurysm, sudden cardiac arrest, and/or stroke. Cardiovascular disease also includes diseases or conditions affecting heart muscles, valves, or rhythm. In embodiments, the cardiovascular disease is coronary artery disease. In embodiments, the cardiovascular disease is high blood pressure. In embodiments, the cardiovascular disease is cardiac arrest. In embodiments, the cardiovascular disease is congestive heart failure. In embodiments, the cardiovascular disease is arrhythmia. In embodiments, the cardiovascular disease is stroke. In embodiments, the cardiovascular disease is peripheral artery disease. In embodiments, the cardiovascular disease is congenital heart disease. In embodiments, the cardiovascular disease is cardiomyopathy. In embodiments, the cardiovascular disease is premature coronary artery disease (CAD). In embodiments, the cardiovascular disease is subclinical atherosclerosis.

“Patient” or “subject in need thereof” refers to a living organism suffering from or prone to a disease or condition that can be treated by administration of a compound, composition, or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Biological sample” refers to materials obtained from or derived from a subject or patient. A biological sample includes sections of tissues such as biopsy (e.g., tumor biopsy) and autopsy samples, and frozen sections taken for histological purposes. Such samples include bodily fluids such as blood and blood fractions or products (e.g., serum, plasma, platelets, red blood cells, circulating tumor cells, and the like), lymph, sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc.

The term “tumor biopsy” refers to tumor tissue sample taken by appropriate means,such as via fine needle biopsy, core needle biopsy, excisional or incisional biopsy, endoscopic biopsy, laparscopic biopsy, thorascopic mediastrinoscopic biopsy, laparotomy, thoracotomy, skin biopsy, and sentinel lymph node mapping and biopsy. Any suitable method for obtaining a tissue sample of a tumor may be used in conjunction with the methods as provided herein.

The term “circulating tumor cell” refers to a cancer cell derived form (e.g. that has detached from) a tumor (e.g. primary tumor). The circulating tumor cell may be circulating in the bloodstream and/or lymphatic system of the subject having the tumor.

A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In embodiments, a biological sample is obtained from a patient. In embodiments, a biological samples is obtained from a normal (non-disease) individual. In embodiments, the sample is obtained from a human.

In embodiments, a biological sample is obtained from a subject (patient) prior to administering a treatment to the subject. Thus, the term “pre-treatment biological sample” refers to a biological sample taken from a patient prior to the patient receiving a treatment (e.g., MDA-7 treatment). The pre-treatment sample may be obtained at any time point prior to the patient receiving a treatment (e.g., MDA-7 treatment). In embodiments, the pre-treatment biological sample is obtained 5, 4, 3, 2, or 1 year prior to the patient receiving a treatment. In embodiments, the pre-treatment biological sample is obtained 11, 10, 9, 8, 7, 6, 5, 4, 3, 2, or 1 month prior to the patient receiving a treatment. In embodiments, the pre-treatment biological sample is obtained 30, 25, 20, 15, 10, 9, 8, 7, 6, 5, 4, 3, 2, or 1 day prior to the patient receiving a treatment. In embodiments, the pre-treatment biological sample is obtained 23, 22, 21, 20, 15, 10, 9, 8, 7, 6, 5, 4, 3, 2, or 1 hour prior to the patient receiving a treatment. In embodiments, the pre-treatment biological sample is obtained 50, 40, 30, 20, 10, 9, 8, 7, 6, 5, 4, 3, 2, or 1 minute prior to the patient receiving a treatment.

In embodiments, a biological sample is obtained from a subject (patient) after administering a treatment to the subject. Thus, the term “post-treatment biological sample” refers to a biological sample taken from a patient after the patient has received a treatment (e.g., MDA-7 treatment). The post-treatment sample may be obtained at any time point after the patient has received a treatment. In embodiments, the post-treatment biological sample is taken 5, 4, 3, 2, or 1 year after the patient has received a treatment. In embodiments, the post-treatment biological sample is obtained 11, 10, 9, 8, 7, 6, 5, 4, 3, 2, or 1 month after the patient has received a treatment. In embodiments, the post-treatment biological sample is obtained 30, 25, 20, 15, 10, 9, 8, 7, 6, 5, 4, 3, 2, or 1 day after the patient has received a treatment. In embodiments, the post-treatment biological sample is obtained 23, 22, 21, 20, 15, 10, 9, 8, 7, 6, 5, 4, 3, 2, or 1 hour after the patient has received a treatment. In embodiments, the post-treatment biological sample is obtained 50, 40, 30, 20, 10, 9, 8, 7, 6, 5, 4, 3, 2, to 1 minute after the patient has received a treatment.

The term “pre-treatment” refers to the time period prior to a treatment and can be refer to, for example, a biological sample, level of protein, level of mRNA, level of microRNA, obtained or detected 5, 4, 3, 2, or 1 year prior to the patient receiving a treatment; 11, 10, 9, 8, 7, 6, 5, 4, 3, 2, or 1 month prior to the patient receiving a treatment; 30, 25, 20, 15, 10, 9, 8, 7, 6, 5, 4, 3, 2, or 1 day prior to the patient receiving a treatment; 23, 22, 21, 20, 15, 10, 9, 8, 7, 6, 5, 4, 3, 2, or 1 hour prior to the patient receiving a treatment; 50, 40, 30, 20, 10, 9, 8, 7, 6, 5, 4, 3, 2, to 1 minute prior to the patient receiving a treatment.

Likewise, the term “post-treatment” refers to the time period after a treatment and can refer to, for example, a biological sample, level of protein, level of mRNA, level of microRNA, obtained or detected 5, 4, 3, 2, or 1 year after the patient has received a treatment; 11, 10, 9, 8, 7, 6, 5, 4, 3, 2, or 1 month after the patient has received a treatment; 30, 25, 20, 15, 10, 9, 8, 7, 6, 5, 4, 3, 2, or 1 day after the patient has received a treatment; 23, 22, 21, 20, 15, 10, 9, 8, 7, 6, 5, 4, 3, 2, or 1 hour after the patient has received a treatment; 50, 40, 30, 20, 10, 9, 8, 7, 6, 5, 4, 3, 2, to 1 minute after the patient has received a treatment.

A “control” or “standard control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). Additionally, a test sample can be taken from a patient suspected of having a given disease (e.g., cancer) and compared to samples from a known disease patient (e.g., cancer patient), or a known normal (non-disease, healthy) individual (e.g., standard control). A control can also represent an average value gathered from a number of tests or results. In embodiments, the average value is attained from testing a population of non-disease individuals. In embodiments, the average value is attained from testing a population of disease patients. In embodiments, the average value is attained from testing a population of disease patients prior to the patients receiving a treatment. In embodiments, the average value is attained from testing a population of disease patients after the patients have received a treatment. One of skill in the art will recognize that controls can be designed for assessment of any number of parameters. For example, a control can be devised to compare therapeutic benefit based on pharmacological data (e.g., half-life) or therapeutic measures (e.g., comparison of side effects). A control value can also be obtained from the same individual, e.g., from an earlier-obtained sample, prior to disease, or prior to treatment (e.g., pre-treatment). One of skill will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “level of miR-221” refers to a level (e.g. an expression level) of miR-221 (i.e., amount of miR-221 molecules) detected in a biological sample. In embodiments, the miR-221 level (i.e., amount of miR-221 molecules) is a miR-221 level detected in a subject suffering from a disease (e.g., cancer, inflammatory disease, infectious disease, autoimmune disease, cardiovascular disease). In embodiments, the level of miR-221 is detected in a pre-treatment or post-treatment biological sample taken from a patient. Thus, in embodiments, the level of miR-221 is a pre-treatment miR-221 level or a post-treatment miR-221 level. In embodiments, the level of miR-221 is detected in a control sample. In embodiments, the control sample is derived from a healthy individual. The pre-treatment or post-treatment level of miR-221 may be compared to a miR-221 level in a control sample obtained from, for example, a non-disease individual. The pre-treatment or post-treatment level of miR-221 may be greater than the level of miR-221 detected in the control sample obtained from the non-diseased individual. In embodiments, the pre-treatment level of miR-221 is at least 1.02, 1.03, 1.04, 1.05, 1.1, 1.2, 1.3, 1.4, 1.5, 1.6, 1.7, 1.8, 1.9, 2, 2.5, 3, 3.5, 4, 4.5, 5, 5.5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, or 300 times greater than the miR-221 level detected in the control sample. In embodiments, the post-treatment level of miR-221 is at least 1.02, 1.03, 1.04, 1.05, 1.1, 1.2, 1.3, 1.4, 1.5, 1.6, 1.7, 1.8, 1.9, 2, 2.5, 3, 3.5, 4, 4.5, 5, 5.5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, or 300 times lower than the pre-treatment miR-221 level.

A standard control may be a level of miR-221 detected in a non-diseased tissue from the same patient. In embodiments, the pre-treatment level of miR-221 is detected in a biological sample from diseased tissue from the same patient. In embodiments, the post-treatment level of miR-221 is detected in a biological sample (e.g., tumor biopsy) from diseased tissue from the same patient.

A standard control may be a level of miR-221 detected in a population of non-diseased individuals. In embodiments, the level of miR-221 is greater than the mean of the level of miR-221 detected in the non-disease population. In embodiments, the level of miR-221 is greater than the median of the level of miR-221 detected in the non-disease population.

A standard control may be a level of miR-221 detected in a population of patients (e.g., patients suffering from a miR-221 and/or beclin-1 associated disease). For example, the standard control may be a level of miR-221 detected in a population of patients suffering from cancer. In embodiments, the level of miR-221 detected in a biological sample is compared to the mean of the level of miR-221 detected in the cancer patient population. In embodiments, the level of miR-221 detected in a biological sample is compared to the median of the level of miR-221 detected in the cancer patient population. Further, a standard control may be a level of miR-221 detected in a population of patients suffering from the same disease (e.g., cancer (e.g., lung cancer, prostate cancer, melanoma, neuroblastoma, etc.) as the subject. In embodiments, the level of miR-221 detected in a biological sample is compared to the mean of the level of miR-221 detected in the cancer patient population having the same type of cancer as the subject. In embodiments, the level of miR-221 detected in a biological sample is compared to the median of the level of miR-221 detected in the cancer patient population having the same type of cancer as the subject.

A cancer “not expressing MDA-7” refers to a cancer which has a reduced expression (e.g. lacks expression) of detectable levels (amounts of protein or RNA) of MDA-7 relative to a standard control. In embodiments, the level of MDA-7 in a sample deemed as not expressing MDA-7 is 0.8, 0.7, 0.6, 0.5, 0.4, 0.3, 0.2, 0.1 0.09, 0.08, 0.7, 0.06, .05, 0.04, 0.03, 0.02, 0.01, 0.005, 0.004, 0.003, 0.002, or 0.001 times less relative to a standard control.

A standard control may be a level of MDA-7 obtained from non-diseased tissue from the patient. The level of MDA-7 detected in a pre-treatment biological sample may be compared to the level of MDA-7 detected in the healthy tissue taken from the same patient. I

A standard control may be a level of MDA-7 obtained from a population of non-disease individuals. In this case, the standard control is the mean or median level of MDA-7 detected in the non-diseased patient population. In embodiments, a disease not expressing MDA-7 has a level less than the mean of the level of MDA-7 detected in the non-disease population. In embodiments, a disease not expressing MDA-7 has a level less than the median of the level of

MDA-7 detected in the non-disease population.

A standard control may be a level of MDA-7 detected in a population of patients (e.g., patients suffering from a miR-221 and/or beclin-1 associated disease). For example, the standard control may be a level of MDA-7 detected in a population of patients suffering from cancer. In embodiments, the level of MDA-7 detected in a biological sample is compared to the mean of the level of MDA-7 detected in the cancer patient population. In embodiments, the level of MDA-7 detected in a biological sample is compared to the median of the level of MDA-7 detected in the cancer patient population. Further, a standard control may be a level of MDA-7 detected in a population of patients suffering from the same disease (e.g., cancer (e.g., lung cancer, prostate cancer, melanoma, neuroblastoma, etc.) as the subject. In embodiments, the level of MDA-7 detected in a biological sample is compared to the mean of the level of MDA-7 detected in the cancer patient population having the same type of cancer as the subject. In embodiments, the level of MDA-7 detected in a biological sample is compared to the median of the level of MDA-7 detected in the cancer patient population having the same type of cancer as the subject.

A cancer “not expressing beclin-1” refers to a cancer which has a reduced expression (e.g. lacks expression of) detectable levels (amounts of protein or RNA) of beclin-1 relative to a standard control. In embodiments, the level of beclin-1 in a sample deemed as not expressing beclin-1 is 0.8, 0.7, 0.6, 0.5, 0.4, 0.3, 0.2, 0.1 0.09, 0.08, 0.7, 0.06, .05, 0.04, 0.03, 0.02, 0.01, 0.005, 0.004, 0.003, 0.002, or 0.001 times less relative to a standard control.

A standard control may be a level of beclin-1 obtained from non-diseased tissue from the patient. The level of beclin-1 detected in a pre-treatment biological sample may be compared to the level of beclin-1 detected in the healthy tissue taken from the same patient. I

A standard control may be a level of beclin-1 obtained from a population of non-disease individuals. In this case, the standard control is the mean or median level of beclin-1 detected in the non-diseased patient population. In embodiments, a disease not expressing beclin-1 has a level less than the mean of the level of beclin-1 detected in the non-disease population. In embodiments, a disease not expressing beclin-1 has a level less than the median of the level of beclin-1 detected in the non-disease population.

A standard control may be a level of beclin-1 detected in a population of patients (e.g., patients suffering from a miR-221 and/or beclin-1 associated disease). For example, the standard control may be a level of beclin-1 detected in a population of patients suffering from cancer. In embodiments, the level of beclin-1 detected in a biological sample is compared to the mean of the level of beclin-1 detected in the cancer patient population. In embodiments, the level of beclin-1 detected in a biological sample is compared to the median of the level of beclin-1 detected in the cancer patient population. Further, a standard control may be a level of beclin-1 detected in a population of patients suffering from the same disease (e.g., cancer (e.g., lung cancer, prostate cancer, melanoma, neuroblastoma, etc.) as the subject. In embodiments, the level of beclin-1 detected in a biological sample is compared to the mean of the level of beclin-1 detected in the cancer patient population having the same type of cancer as the subject. In embodiments, the level of beclin-1 detected in a biological sample is compared to the median of the level of beclin-1 detected in the cancer patient population having the same type of cancer as the subject.

The term “associated” or “associated with” in the context of a substance or substance activity, function, or level (i.e., amount of substance) associated with a disease (e.g. a miR-221 and/or beclin-1 protein associated disease, a cancer associated with miR-221 and/or beclin-1 protein activity, a miR-221 and/or beclin-1 protein associated cancer, a miR-221 and/or beclin-1 protein associated disease (e.g., cancer, inflammatory disease, autoimmune disease, infectious disease, or cardiovascular disease)) means that the disease (e.g. cancer, inflammatory disease, autoimmune disease, or infectious disease, or cardiovascular disease) is caused by (in whole or in part), or a symptom of the disease is caused by (in whole or in part) the substance or substance activity, function, or level (i.e., amount of substance). For example, a cancer associated with miR-221 and/or beclin-1 activity, function, or level (i.e., amount of substance) may be a cancer that results (entirely or partially) from aberrant miR-221 and/or beclin-1 function (e.g. enzyme activity, protein-protein interaction, signaling pathway) or a cancer wherein a particular symptom of the disease is caused (entirely or partially) by aberrant miR-221 and/or beclin-1 activity, function, or level (e.g., amount). As used herein, what is described as being associated with a disease, if a causative agent, could be a target for treatment of the disease. For example, a cancer associated with miR-221 and/or beclin-1 activity, function, or level (i.e., amount of substance) or a miR-221 and/or beclin-1 associated disease (e.g., cancer, inflammatory disease, autoimmune disease, cardiovascular disease, or infectious disease), may be treated with MDA-7, in the instance where aberrant miR-221 and/or beclin-1 activity, function (e.g. signaling pathway activity), or level (i.e., amount of substance) causes the disease (e.g., cancer, inflammatory disease, autoimmune disease, or infectious disease).

The term “aberrant” as used herein refers to different from normal. When used to describe enzymatic activity, microRNA activity, or protein activity, aberrant refers to activity, function, or level (i.e., amount of substance) that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms. Aberrant activity may refer to a level of a substance (i.e., amount of substance) that results in a disease, wherein returning the aberrant level to a normal or non-disease-associated level (e.g. by administering a compound or using a method as described herein), results in reduction of the disease or one or more disease symptoms. In embodiments, miR-221 level is upregulated in the miR-221 and/or beclin-1 associated disease. In embodiments, beclin-1 level is downregulated in the miR-221 and/or beclin-1 associated disease.

A level (i.e., amount of substance (e.g., miR-221, beclin-1)) may be detected (e.g., identified and/or quantified) using methods of detecting nucleic acids (e.g., microRNA, mRNA) and proteins well known in the art. For example, nucleic acids may be detected using nucleic acid hybridization methods that make use of complementary probes or primers that hybridize to specific nucleic acid sequences. Nucleic acid hybridization methods can be used to identify small amounts of DNA or RNA (e.g., after reverse transcription to create DNA from the RNA) in PCR (e.g., real-time PCR, also known as quantitative PCR (qPCR); single cell PCR). Alternatively, hybridizing probes, including probes that include a detectable moiety (e.g., fluorescently labeled, radioactively labeled) can be used to detect DNA in Southern blotting, for the detection of genes or to detect RNA in Northern blotting. Detection can be made in biological samples, for example, tumor biopsy or blood samples. Detection of DNA or RNA using hybridizing probes in an intact cell (e.g., tumor cell, circulating tumor cell) or tissue sample (e.g., tumor biopsy) can be accomplished via in situ hybridization. In in situ hybridization, probes including a detectable moieties are allowed to hybridize with nucleic acids (e.g., RNA, DNA) residing in an intact cell (e.g., tumor cell, circulating tumor cell) or tissue sample (e.g., tumor biopsy) and subsequently analyzed (e.g., quantified) by microscope examination. Non-limiting examples for performing in situ hybridization are disclosed in McFadden Meth in Cell Biol, 1995; Jensen E. The Anatomical Review, 2014; Ratan et al., Cureus 2017, which are incorporated by reference in their entirety.

Alternatively, a biological sample may be further processed to produce, for example, cellular extracts including RNA, DNA, protein. Cellular extracts may be further purified to isolate DNA, RNA, or protein. Isolated DNA can be used in Southern blotting analysis while, RNA can be used in Northern blotting analysis to determine the presence and amount of the DNA or RNA of interest. As stated above, small amounts of isolated DNA or RNA, for example amount of DNA or RNA taken from a single cell, can be identified using PCR, for example real time PCR which allows quantification of the amount of DNA or RNA in the sample. Non-limiting examples of methods for performing real time PCR are disclosed in Heid et al., Genome Res, 1996; US 2009/00537261 Al, which are hereby incorporated by reference in their entirety.

Non-limiting examples of methods for detecting total protein in a sample include absorbance measures, Bradford protein assay, Biuret test derived assays including bicinchoninic acid assay and Lowry protein assay, fluorescamine, amido black, colloidal gold, and nitrogen detection methods including the Kjeldahl method and Dumas method. Non-limiting examples of methods for detecting a single protein in a sample include spectrometry methods, including high-performance liquid chromatography (HPLC), liquid chromatography-mass spectrometry (LC/MS); antibody dependent methods, including enzyme-linked immunosorbent assay (ELISA), protein immunoprecipitation, immunoelectrophoresis, protein immunostaining, and Western blotting analysis. Western blotting analysis, sometimes referred to as protein immunoblotting, is useful for detecting and quantifying a single protein. Non-limiting examples for performing Western blotting analysis are disclosed in Kurien BT and Scofield RH, Methods 2006; Mahmood T and Yang P-C, N Am J Med Sci 2012, which are incorporated by reference in their entirety. It should also be appreciated that protein levels may be determined by use of PCR, e.g., real time PCR, by isolating mRNA and converting the mRNA to DNA via reverse transcriptase.

Any suitable method of detecting a level of nucleic acid or protein in a biological sample may be used in conjunction with the methods described herein, including embodiments thereof. In embodiments, a level is detected in a biological sample obtained from a patient prior to the patient being administered a treatment (i.e., a pre-treatment biological sample). In this case, the level is referred to as a “pre-treatment level.” In embodiments, a level is detected in biological samples obtained from a population of patients prior to the patients receiving a treatment. In embodiments, a level is detected in a biological sample obtained from a patient after the patient has been administered a treatment (i.e., a post-treatment biological sample). In this case, the level is referred to as a “post-treatment level.” In embodiments, a level is detected in biological samples obtained from a population of patients after the patients have received a treatment. In embodiments, a level is detected in a biological sample obtained from a non-disease individual. In embodiments, a level is detected in biological samples obtained from a population of non-disease individuals.

The terms “inhibitor,” “repressor,” “antagonist” or “downregulator” interchangeably refer to a substance capable of detectably decreasing the expression or activity of a given gene or protein relative to the absence of the “inhibitor,” “repressor,” “antagonist” or “downregulator”. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components. For example, delivery of an MDA-7 treatment may act to downregulate miR-221, and upregulate beclin-1. Thus, MDA-7 treatment may act on miR-221 to modulate downstream effectors or signaling pathway components (e.g., upregulate beclin-1, MMP, TIMP3, BMP2, secreted uPAR isoform2), resulting in changes in cell growth, proliferation, or survival.

As used herein, the terms “pharmaceutically” acceptable is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present invention, the dose will generally refer to the amount of pulmonary disease treatment, anti-inflammatory agent, agonist or antagonist. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical, and depends on the route of administration. For example, a dosage form can be in a liquid form for nebulization, e.g., for inhalants, in a tablet or liquid, e.g., for oral delivery, or a saline solution, e.g., for injection.

As used herein, the terms “treat” and “prevent” are not intended to be absolute terms. Treatment can refer to any delay in onset, reduction in the frequency or severity of symptoms, amelioration of symptoms, improvement in patient comfort and/or respiratory function, etc. The effect of treatment can be compared to an individual or pool of individuals not receiving a given treatment, or to the same patient prior to, or after cessation of, treatment.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, treating or treatment does not include prophylactic treatment.

The term “MDA-7 treatment” refers to administering of an effective amount of MDA-7 to a subject, such as patient in need thereof, a cancer patient or a patient having a disease associated with aberrant miR-221 and/or beclin-1 (e.g., cancer, inflammatory disease, infectious disease, autoimmune disease, or cardiovascular disease).

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cancer cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The compositions described herein can be used in combination with one another, with other active agents known to be useful in treating a cancer such as anti-cancer agents.

Examples of anti-cancer agents include, but are not limited to, radiation, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol;

cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim;

finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides;

insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin (including recombinant interleukin II, or r1L.sub.2), interferon alfa-2a; interferon alfa-2b; interferon alfa-nl; interferon alfa-n3; interferon beta-la; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin;

zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), Cryptophycin 52 (i.e. LY-355703), Vitilevuamide, Tubulysin A, Canadensol, Centaureidin (i.e. NSC-106969), Oncocidin Al (i.e. BTO-956 and DIME), Fijianolide B, Laulimalide, Narcosine (also known as NSC-5366), Nascapine, Vanadocene acetylacetonate, T-138026 (Tularik), Monsatrol, lnanocine (i.e. NSC-698666), Eleutherobins (such as Desmethyleleutherobin, Desaetyleleutherobin, lsoeleutherobin A, and Z-Eleutherobin), Caribaeoside, Caribaeolin, Halichondrin B, Diazonamide A, Taccalonolide A, Diozostatin, (-)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™) erlotinib (Tarceva™), cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), 5-FU, MCL-1 inhibitor, ROS inducer, sorafenib, imatinib, sunitinib, dasatinib, or the like. In embodiments, the compositions herein may be used in combination with adjunctive agents that may not be effective alone, but may contribute to the efficacy of the active agent in treating cancer.

“Chemotherapeutic” or “chemotherapeutic agent” is used in accordance with its plain ordinary meaning and refers to a chemical composition or compound having antineoplastic properties or the ability to inhibit the growth or proliferation of cells.

An “ROS inducer” refers to compounds or compositions useful for increasing reactive oxygen species.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

II. Methods of Monitoring

Provided herein are, inter alia, methods of monitoring levels of miR-221 and beclin-1 in patients being treated for miR-221- and/or beclin-l-associated diseases (e.g., cancer, inflammatory disease, infectious disease, autoimmune disease, cardiovascular disease). Upregulated levels of microRNA miR-221, a known oncomir (i.e., cancer promoting microRNA) that targets and degrades tumor suppressor mRNAs have been observed in various cancers and other diseases. In contrast, decreased levels of autophagy promoter beclin-1 have been detected in a number of cancers. Applicants have identified beclin-1 as a new transcriptional target of miR-221 and show that levels of beclin-1 in miR-221 associated diseases are decreased.

Applicants further show that treatment of cancer with melanoma differentiation associated gene-7/Interleukin-24 (MDA-7/IL-24), which displays broad spectrum anti-cancer activity without harming normal cells or tissues, downregulates miR-221 and consequently upregulates beclin-1 expression levels. The methods provided herein are therefore useful, inter alia, to monitor and determine treatment efficacy by determining (detecting) levels of miR-221, beclin-1, a combination thereof or of molecules downstream of the miR-221 or beclin-1 signaling pathways, in patients receiving, having received or to be received MDA-7 treatment.

Thus, in an aspect is provided a method of detecting a miR-221 level in a cancer patient, wherein the cancer patient has received a MDA-7 treatment, the method including: (i) obtaining a post-treatment biological sample from the cancer patient; and (ii) detecting a post-treatment level of miR-221 in the post-treatment biological sample. “MDA-7 treatment” as provided herein refers to administering to a subject in need thereof a therapeutically effective amount of a MDA-7 protein or vector encoding the same.

In embodiments, the post-treatment biological sample is a tumor biopsy. In embodiments, the post-treatment biological sample is a blood sample. In embodiments, the post-treatment biological sample includes a circulating tumor cell. In embodiments, the post-treatment biological sample is a circulating tumor cell.

In embodiments, the detecting includes performing real-time PCR. In embodiments, the detecting includes performing in situ hybridization.

In embodiments, the method further includes detecting a post-treatment level of beclin-1 in the post-treatment biological sample.

In embodiments, the detecting includes performing real-time PCR. In embodiments, the detecting includes performing Western blotting analysis.

As mentioned above, detecting a level of miR-221 may be accomplished by detecting a level of a downstream target (i.e., a target the expression of which is directly or indirectly regulated by miR-221) of miR-221. Downstream targets of miR-221 include without limitation MMP (i.e., the family of matrix metalloproteinases), TIMP3, BMP2, and secreted uPAR isoform2. Therefore, in embodiments, the detecting a post-treatment level of miR-221 includes detecting a post-treatment level of MMP, a post-treatment level of TIMP3, a post-treatment level of BMP2, a post-treatment level of secreted uPAR isoform2 or a combination thereof in the post-treatment biological sample. In embodiments, the detecting a post-treatment level of miR-221 includes detecting a post-treatment level of MMP in the post-treatment biological sample. In embodiments, the detecting a post-treatment level of miR-221 includes detecting a post-treatment level of TIMP3. In embodiments, the detecting a post-treatment level of miR-221 includes detecting a post-treatment level of BMP2 in the post-treatment biological sample. In embodiments, the detecting a post-treatment level of miR-221 includes detecting a post-treatment level of secreted uPAR isoform2 in the post-treatment biological sample. In embodiments, the detecting a post-treatment level of miR-221 includes detecting a post-treatment level of MMP, a post-treatment level of TIMP3, a post-treatment level of BMP2 and a post-treatment level of secreted uPAR isoform2 in the post-treatment biological sample.

In embodiments, the detecting a post-treatment level of miR-221 includes detecting a post-treatment level of miR-22, a post-treatment level of MMP, a post-treatment level of TIMP3, a post-treatment level of BMP2, a post-treatment level of secreted uPAR isoform2 or a combination thereof in the post-treatment biological sample. In embodiments, the detecting a post-treatment level of miR-221 includes detecting a post-treatment level of miR-22, a post-treatment level of MMP, a post-treatment level of TIMP3, a post-treatment level of BMP2 and a post-treatment level of secreted uPAR isoform2 in the post-treatment biological sample.

In embodiments, the method further includes (i) obtaining a pre-treatment biological sample from the cancer patient prior to the cancer patient receiving a MDA-7 treatment; and (ii) detecting a pre-treatment level of miR-221 in the pre-treatment biological sample. The pre-treatment level may be detected in a non-diseased tissue of the patient or in a diseased tissue (e.g. tumor biopsy).

In embodiments, the pre-treatment biological sample is a tumor biopsy. In embodiments, the pre-treatment biological sample is a blood sample. In embodiments, the pre-treatment biological sample includes a circulating tumor cell. In embodiments, the pre-treatment biological sample is a circulating tumor cell.

In embodiments, the detecting includes performing real-time PCR. In embodiments, the detecting includes performing in situ hybridization.

In embodiments, the post-treatment level of miR-221 detected in the post-treatment biological sample is compared to the pre-treatment level of miR-221 detected in the pre-treatment biological sample. In embodiments, the post-treatment level of miR-221 is decreased relative to the pre-treatment level of miR-221. In embodiments, the post-treatment level of miR-221 is increased relative to the pre-treatment level. In embodiments, the post-treatment level of miR-221 is essentially the same relative to the pre-treatment level of miR-221.

In embodiments, the detecting a pre-treatment level of miR-221 includes detecting a pre-treatment level of MMP, a pre-treatment level of TIMP3, a pre-treatment level of BMP2, a pre-treatment level of secreted uPAR isoform2 or any combination thereof in the pre-treatment biological sample. In embodiments, the detecting a pre-treatment level of miR-221 includes detecting a pre-treatment level of MMP in the pre-treatment biological sample. In embodiments, the detecting a pre-treatment level of miR-221 includes detecting a pre-treatment level of TIMP3 in the pre-treatment biological sample. In embodiments, the detecting a pre-treatment level of miR-221 includes a pre-treatment level of secreted uPAR isoform2 in the pre-treatment biological sample. In embodiments, the detecting a pre-treatment level of miR-221 includes detecting a pre-treatment level of MMP, a pre-treatment level of TIMP3, a pre-treatment level of BMP2, and a pre-treatment level of secreted uPAR isoform2 in the pre-treatment biological sample. In embodiments, the detecting a pre-treatment level of miR-221 includes detecting a pre-treatment level of miR-221, a pre-treatment level of MMP, a pre-treatment level of TIMP3, a pre-treatment level of BMP2, a pre-treatment level of secreted uPAR isoform2 or any combination thereof in the pre-treatment biological sample. In embodiments, the detecting a pre-treatment level of miR-221 includes detecting a pre-treatment level of miR-221, a pre-treatment level of MMP, a pre-treatment level of TIMP3, a pre-treatment level of BMP2 and a pre-treatment level of secreted uPAR isoform2 in the pre-treatment biological sample.

In embodiments, the post-treatment level of MMP detected in the post-treatment biological sample is compared to the pre-treatment level of MMP in the pre-treatment biological sample. In embodiments, the post-treatment level of MMP is increased compared to the pre-treatment level of MMP. In embodiments, the post-treatment level of MMP is decreased relative to the pre-treatment level of MMP. In embodiments, the post-treatment level of MMP is essentially the same relative to the pre-treatment level of MMP.

In embodiments, the post-treatment level of TIMP3 detected in the post-treatment biological sample is compared to the pre-treatment level of TIMP3 in the pre-treatment biological sample. In embodiments, the post-treatment level of TIMP3 is increased compared to the pre-treatment level of TIMP3. In embodiments, the post-treatment level of TIMP3 is decreased relative to the pre-treatment level of TIMP3. In embodiments, the post-treatment level of TIMP3 is essentially the same relative to the pre-treatment level of TIMP3.

In embodiments, the post-treatment level of BIMP2 detected in the post-treatment biological sample is compared to the pre-treatment level of BIMP2 in the pre-treatment biological sample. In embodiments, the post-treatment level of BIMP2 is increased compared to the pre-treatment level of BIMP2. In embodiments, the post-treatment level of TIMP3 is decreased relative to the pre-treatment level of BIMP2. In embodiments, the post-treatment level of TIMP3 is essentially the same relative to the pre-treatment level of BIMP2.

In embodiments, the post-treatment level of uPAR isoform2 detected in the post-treatment biological sample is compared to the pre-treatment level of uPAR isoform2 in the pre-treatment biological sample. In embodiments, the post-treatment level of uPAR isoform2 is increased compared to the pre-treatment level of uPAR isoform2. In embodiments, the post-treatment level of uPAR isoform2 is decreased relative to the pre-treatment level of uPAR isoform2. In embodiments, the post-treatment level of uPAR isoform2 is essentially the same relative to the pre-treatment level of uPAR isoform2.

In embodiments, the cancer patient has been further treated with an additional anti-cancer agent. In embodiments, the additional anti-cancer agent is not MDA-7. In embodiments, the additional anti-cancer agent is a ROS inducer. Non-limiting examples of ROS inducers include arsenic trioxide, hydrogen peroxide, or pyocyanin. Thus, in embodiments, the ROS inducer is arsenic trioxide, hydrogen peroxide, or pyocyanin. In embodiments, the ROS inducer is arsenic trioxide. In embodiments, the ROS inducer is hydrogen peroxide. In embodiments, the ROS inducer is pyocyanin.

In embodiments, the ROS inducer is delivered at a low dose. In embodiments, a low dose is about 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 200 μM, 300 μM, 400 μM, 500 μM, 600 μM, 700 μM, 800 μM, 900 μM, or 1 mM. In embodiments, a low dose is about 0.01 μM. In embodiments, a low dose is about 0.05 μM. In embodiments, a low dose is about 0.1 μM. In embodiments, a low dose is about 0.2 μM. In embodiments, a low dose is about 0.3 μM. In embodiments, a low dose is about 0.4 μM. In embodiments, a low dose is about 0.5 μM. In embodiments, a low dose is about 0.6 μM. In embodiments, a low dose is about 0.7 μM. In embodiments, a low dose is about 0.8 μM, 0.9 μM. In embodiments, a low dose is about 1 μM. In embodiments, a low dose is about 5 μM. In embodiments, a low dose is about 10 μM. In embodiments, a low dose is about 15 μM, 20 μM. In embodiments, a low dose is about 25 μM. In embodiments, a low dose is about 30 μM. In embodiments, a low dose is about 35 μM. In embodiments, a low dose is about 40 μM. In embodiments, a low dose is about 45 μM. In embodiments, a low dose is about 50 μM. In embodiments, a low dose is about 55 μM. In embodiments, a low dose is about 60 μM. In embodiments, a low dose is about 65 μM. In embodiments, a low dose is about 70 μM. In embodiments, a low dose is about 75 μM. In embodiments, a low dose is about 80 μM. In embodiments, a low dose is about 85 μM. In embodiments, a low dose is about 90 μM. In embodiments, a low dose is about 95 μM. In embodiments, a low dose is about 100 μM. In embodiments, a low dose is about 200 μM. In embodiments, a low dose is about 300 μM. In embodiments, a low dose is about 400 μM. In embodiments, a low dose is about 500 μM. In embodiments, a low dose is about 600 μM. In embodiments, a low dose is about 700 μM. In embodiments, a low dose is about 800 μM. In embodiments, a low dose is about 900 μM. In embodiments, a low dose is about 1 mM.

In embodiments, a low dose is 0.01 μM. In embodiments, a low dose is 0.05 μM. In embodiments, a low dose is 0.1 μM. In embodiments, a low dose is 0.2 μM. In embodiments, a low dose is 0.3 μM. In embodiments, a low dose is 0.4 μM. In embodiments, a low dose is 0.5 μM. In embodiments, a low dose is 0.6 μM. In embodiments, a low dose is 0.7 μM. In embodiments, a low dose is 0.8 μM, 0.9 μM. In embodiments, a low dose is 1 μM. In embodiments, a low dose is 5 μM. In embodiments, a low dose is 10 μM. In embodiments, a low dose is 15 μM, 20 μM. In embodiments, a low dose is 25 μM. In embodiments, a low dose is 30 μM. In embodiments, a low dose is 35 μM. In embodiments, a low dose is 40 μM. In embodiments, a low dose is 45 μM. In embodiments, a low dose is 50 μM. In embodiments, a low dose is 55 μM. In embodiments, a low dose is 60 μM. In embodiments, a low dose is 65 μM. In embodiments, a low dose is 70 μM. In embodiments, a low dose is 75 μM. In embodiments, a low dose is 80 μM. In embodiments, a low dose is 85 μM. In embodiments, a low dose is 90 μM. In embodiments, a low dose is 95 μM. In embodiments, a low dose is 100 μM. In embodiments, a low dose is 200 μM. In embodiments, a low dose is 300 μM. In embodiments, a low dose is 400 μM. In embodiments, a low dose is 500 μM. In embodiments, a low dose is 600 μM. In embodiments, a low dose is 700 μM. In embodiments, a low dose is 800 μM. In embodiments, a low dose is 900 μM. In embodiments, a low dose is 1 mM.

In embodiments, the MDA-7 treatment and the additional anti-cancer agent are administered in a combined synergistic amount.

A “combined synergistic amount” as used herein refers to the sum of a first amount of a first agent (e.g., an amount of MDA-7) and a second amount of a second agent (e.g., an anti-cancer agent (e.g., ROS inducer)), that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

In embodiments, a combined synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the first amount (e.g., MDA-7) when used separately from the second amount (e.g., an anti-cancer agent (e.g., ROS inducer)). In embodiments, a combined synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the second amount (e.g., an anti-cancer agent (e.g., ROS inducer)) when used separately from the first amount (e.g., MDA-7).

In embodiments, the cancer patient has melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer. In embodiments, the cancer patient has melanoma. In embodiments, the cancer patient has prostate cancer. In embodiments, the cancer patient has neuroblastoma. In embodiments, the cancer patient has osteosarcoma. In embodiments, the cancer patient has renal carcinoma. In embodiments, the cancer patient has leukemia. In embodiments, the cancer patient has epithelial cancer. In embodiments, the cancer patient has pancreatic cancer. In embodiments, the cancer patient has glioblastoma. In embodiments, the cancer patient has thyroid papillary carcinoma. In embodiments, the cancer patient has esophageal squamous cell carcinoma. In embodiments, the cancer patient has breast cancer. In embodiments, the cancer patient has hepatocellular carcinoma. In embodiments, the cancer patient has liver cancer. In embodiments, the cancer patient has lung cancer. In embodiments, the cancer patient being treated has a metastatic cancer.

In an aspect is provided a method of detecting a beclin-1 level in a cancer patient, wherein the cancer patient has received a MDA-7 treatment, the method including: (i) obtaining a post-treatment biological sample from the cancer patient; and (ii) detecting a post-treatment level of beclin-1 in the post-treatment biological sample.

In embodiments, the post-treatment biological sample is a tumor biopsy. In embodiments, the post-treatment biological sample is a blood sample. In embodiments, the post-treatment biological sample includes a circulating tumor cell. In embodiments, the post-treatment biological sample is a circulating tumor cell. In embodiments, the detecting includes performing real-time PCR. In embodiments, the detecting includes performing Western blotting analysis.

In embodiments, the method further includes detecting a post-treatment level of miR-221 in the post-treatment biological sample. In embodiments, the detecting includes performing real-time PCR. In embodiments, the detecting includes performing in situ hybridization.

Detecting a level of beclin-1 may be accomplished by detecting a level of a downstream target of miR-221. Thus, in embodiments, the detecting a post-treatment level of beclin-1 includes detecting a post-treatment level of MMP, a post-treatment level of TIMP3, a post-treatment level of BMP2, a post-treatment level of secreted uPAR isoform2 or any combination thereof in the post-treatment biological sample. In embodiments, the detecting a post-treatment level of beclin-1 includes detecting a post-treatment level of MMP in the post-treatment biological sample. In embodiments, the detecting a post-treatment level of beclin-1 includes detecting a post-treatment level of TIMP3 in the post-treatment biological sample. In embodiments, the detecting a post-treatment level of beclin-1 includes detecting a post-treatment level of BMP2 in the post-treatment biological sample. In embodiments, the detecting a post-treatment level of beclin-1 includes detecting a post-treatment level of secreted uPAR isoform2 in the post-treatment biological sample. In embodiments, the detecting a post-treatment level of beclin-1 includes detecting a post-treatment level of MMP, a post-treatment level of TIMP3, a post-treatment level of BMP2, and a post-treatment level of secreted uPAR isoform2 in the post-treatment biological sample. In embodiments, the detecting a post-treatment level of beclin-1 includes detecting a post-treatment level of beclin-1, a post-treatment level of MMP, a post-treatment level of TIMP3, a post-treatment level of BMP2, a post-treatment level of secreted uPAR isoform2 or any combination threof in the post-treatment biological sample. In embodiments, the detecting a post-treatment level of beclin-1 includes detecting a post-treatment level of beclin-1, a post-treatment level of MMP, a post-treatment level of TIMP3, a post-treatment level of BMP2, and a post-treatment level of secreted uPAR isoform2 in the post-treatment biological sample.

In embodiments, the method further includes further includes: (i) obtaining a pre-treatment biological sample from the cancer patient prior to the cancer patient receiving a MDA-7 treatment; and (ii) detecting a pre-treatment level of beclin-1 in the pre-treatment biological sample.

In embodiments, the pre-treatment biological sample is a tumor biopsy. In embodiments, the pre-treatment biological sample is a blood sample. In embodiments, the pre-treatment biological sample includes a circulating tumor cell. In embodiments, the pre-treatment biological sample is a circulating tumor cell. In embodiments, the detecting includes performing real-time PCR. In embodiments, the detecting includes performing Western blotting analysis.

In embodiments, the post-treatment level of beclin-1 detected in the post-treatment biological sample is relative to the pre-treatment level of beclin-1 detected in the pre-treatment biological sample. In embodiments, the post-treatment level of beclin-1 is decreased relative to the pre-treatment level of beclin-1. In embodiments, the post-treatment level of beclin-1 is essentially the same relative to the pre-treatment level of beclin-1.

In embodiments, the detecting a pre-treatment level of beclin-1 includes detecting a pre-treatment level of MMP, a pre-treatment level of TIMP3, a pre-treatment level of BMP2, a pre-treatment level of secreted uPAR isoform2 or a combination thereof in the pre-treatment biological sample. In embodiments, the detecting a pre-treatment level of beclin-1 includes detecting a pre-treatment level of MMP, a pre-treatment level of TIMP3, a pre-treatment level of BMP2 and a pre-treatment level of secreted uPAR isoform2 in the pre-treatment biological sample. In embodiments, the detecting a pre-treatment level of beclin-1 includes detecting a pre-treatment level of MMP in the pre-treatment biological sample. In embodiments, the detecting a pre-treatment level of beclin-1 includes detecting a pre-treatment level of TIMP3 in the pre-treatment biological sample. In embodiments, the detecting a pre-treatment level of beclin-1 includes detecting a pre-treatment level of BMP2 in the pre-treatment biological sample. In embodiments, the detecting a pre-treatment level of beclin-1 includes detecting a pre-treatment level of secreted uPAR isoform2 in the pre-treatment biological sample. In embodiments, the detecting a pre-treatment level of beclin-1 includes detecting a pre-treatment level of beclin-1, a pre-treatment level of MMP, a pre-treatment level of TIMP3, a pre-treatment level of BMP2, a pre-treatment level of secreted uPAR isoform2 or a combination thereof in the pre-treatment biological sample. In embodiments, the detecting a pre-treatment level of beclin-1 includes detecting a pre-treatment level of beclin-1, a pre-treatment level of MMP, a pre-treatment level of TIMP3, a pre-treatment level of BMP2, and a pre-treatment level of secreted uPAR isoform2 in the pre-treatment biological sample.

In embodiments, the post-treatment level of MMP detected in the post-treatment biological sample is compared to the pre-treatment level of MMP in the pre-treatment biological sample. In embodiments, the post-treatment level of MMP is increased relative to the pre-treatment MMP level. In embodiments, the post-treatment level of MMP is decreased relative to the pre-treatment MMP level. In embodiments, the post-treatment level of MMP is essentially the same relative to the pre-treatment MMP level.

In embodiments, the post-treatment level of TIMP3 detected in the post-treatment biological sample is compared to the pre-treatment level of TIMP3 in the pre-treatment biological sample. In embodiments, the post-treatment level of TIMP3 is increased relative to the pre-treatment TIMP3 level. In embodiments, the post-treatment level of TIMP3 is decreased relative to the pre-treatment TIMP3 level. In embodiments, the post-treatment level of TIMP3 is essentially the same relative to the pre-treatment TIMP3 level.

In embodiments, the post-treatment level of BMP2 detected in the post-treatment biological sample is compared to the pre-treatment level of BMP2 in the pre-treatment biological sample. In embodiments, the post-treatment level of BMP2 is increased relative to the pre-treatment BMP2 level. In embodiments, the post-treatment level of BMP2 is decreased relative to the pre-treatment BMP2 level. In embodiments, the post-treatment level of BMP2 is essentially the same relative to the pre-treatment BMP2 level.

In embodiments, the post-treatment level of secreted uPAR isoform2 detected in the post-treatment biological sample is compared to the pre-treatment level of secreted uPAR isoform2 in the pre-treatment biological sample. In embodiments, the post-treatment level of uPAR isoform2 is increased relative to the pre-treatment uPAR isoform2 level. In embodiments, the post-treatment level of uPAR isoform2 is decreased relative to the pre-treatment uPAR isoform2 level. In embodiments, the post-treatment level of uPAR isoform2 is essentially the same relative to the pre-treatment uPAR isoform2 level.

In embodiments, the cancer patient has been further treated with an additional anti-cancer agent. In embodiments, the additional anti-cancer agent is not MDA-7. In embodiments, the additional anti-cancer agent is a ROS inducer. Non-limiting examples of in clinic ROS inducers include arsenic trioxide, hydrogen peroxide, or pyocyanin. Thus, in embodiments, the ROS inducer is arsenic trioxide, hydrogen peroxide, or pyocyanin. In embodiments, the ROS inducer is arsenic trioxide. In embodiments, the ROS inducer is hydrogen peroxide. In embodiments, the ROS inducer is pyocyanin.

In embodiments, the ROS inducer is delivered at a low dose. As described above a low dose may be about 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 200 μM, 300 μM, 400 μM, 500 μM, 600 μM, 700 μM, 800 μM, 900 μM, 1 mM.

As described herein the MDA-7 treatment and the additional anti-cancer agent may be administered in a combined synergistic amount.

In embodiments, the cancer patient has melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer. In embodiments, the cancer patient has melanoma. In embodiments, the cancer patient has prostate cancer. In embodiments, the cancer patient has neuroblastoma. In embodiments, the cancer patient has osteosarcoma. In embodiments, the cancer patient has renal carcinoma. In embodiments, the cancer patient has leukemia. In embodiments, the cancer patient has epithelial cancer. In embodiments, the cancer patient has pancreatic cancer. In embodiments, the cancer patient has glioblastoma. In embodiments, the cancer patient has thyroid papillary carcinoma. In embodiments, the cancer patient has esophageal squamous cell carcinoma. In embodiments, the cancer patient has breast cancer. In embodiments, the cancer patient has hepatocellular carcinoma. In embodiments, the cancer patient has liver cancer. In embodiments, the cancer patient has lung cancer. In embodiments, the cancer patient being treated has a metastatic cancer.

III. Methods of Treating Cancer

It is contemplated herein that downregulating miR-221, thereby upregulating beclin-1, via MDA-7 treatment is useful for treating cancers expressing miR-221 and not expressing MDA-7. Thus in an aspect is provided a method of cancer in a subject in need thereof, wherein the subject has a cancer expressing miR-221 and not expressing MDA-7, the method including administering to the subject an effective amount of MDA-7.

In embodiments, the cancer does not express beclin-1.

In embodiments, the method further includes, prior to administering the effective amount of MDA-7: (i) obtaining a pre-treatment biological sample from the subject; and (ii) detecting a pre-treatment level of miR-221 in the pre-treatment biological sample.

In embodiments, the pre-treatment biological sample is a tumor biopsy. In embodiments, the pre-treatment biological sample is a blood sample. In embodiments, the pre-treatment biological sample includes a circulating tumor cell. In embodiments, the pre-treatment biological sample is a circulating tumor cell. In embodiments, the detecting includes performing real-time PCR. In embodiments, the detecting includes performing in situ hybridization.

In embodiments, the pre-treatment level of miR-221 in the pre-treatment biological sample is compared to a standard control. In embodiments, the pre-treatment level of miR-221 is increased relative to the standard control. In embodiments, the pre-treatment level of miR-221 is decreased relative to the standard control. In embodiments, the pre-treatment level of miR-221 is essentially the same relative to the standard control. In embodiments, the standard control is a median level of miR-221 obtained from a population of non-disease individuals. In embodiments, the standard control is a level of miR-221 obtained from non-disease tissue obtained from the patient. In embodiments, the standard control is a mean level of miR-221 obtained from a population of patients having a miR-221 and/or miR-221 associated disease (e.g., cancer). In embodiments, the standard control is a median level of miR-221 obtained from a population of patients having a miR-221 and/or miR-221 associated disease (e.g., cancer).

Similar to the embodiments described above, the detecting a pre-treatment level of miR-221 may include detecting a pre-treatment level of MMP, a pre-treatment level of TIMP3, a pre-treatment level of BMP2, a pre-treatment level of secreted uPAR isoform2 or any combination thereof in the pre-treatment biological sample.

In embodiments, the pre-treatment level of MMP in the pre-treatment biological sample is compared to a standard control. In embodiments, the pre-treatment level of MMP is decreased relative to the standard control. In embodiments, the pre-treatment level of MMP is increased relative to the standard control. In embodiments, the pre-treatment level of MMP is essentially the same relative to the standard control. In embodiments, the standard control is a median level of MMP obtained from a population of non-disease individuals. In embodiments, the standard control is a level of MMP obtained from non-disease tissue obtained from the patient. In embodiments, the standard control is a mean level of MMP obtained from a population of patients having a MMP and/or MMP associated disease (e.g., cancer). In embodiments, the standard control is a median level of MMP obtained from a population of patients having a MMP and/or MMP associated disease (e.g., cancer).

In embodiments, the pre-treatment level of TIMP3 in the pre-treatment biological sample is compared to a standard control. In embodiments, the pre-treatment level of TIMP3 is decreased relative to the standard control. In embodiments, the pre-treatment level of TIMP3 is increased relative to the standard control. In embodiments, the pre-treatment level of TIMP3 is essentially the same relative to the standard control. In embodiments, the standard control is a median level of TIMP3 obtained from a population of non-disease individuals. In embodiments, the standard control is a level of TIMP3 obtained from non-disease tissue obtained from the patient. In embodiments, the standard control is a mean level of TIMP3 obtained from a population of patients having a TIMP3 and/or TIMP3 associated disease (e.g., cancer). In embodiments, the standard control is a median level of TIMP3 obtained from a population of patients having a TIMP3 and/or TIMP3 associated disease (e.g., cancer).

In embodiments, the pre-treatment level of BMP2 in the pre-treatment biological sample is compared to a standard control. In embodiments, the pre-treatment level of BMP2 is decreased relative to the standard control. In embodiments, the pre-treatment level of BMP2 is increased relative to the standard control. In embodiments, the pre-treatment level of BMP2 is essentially the same relative to the standard control. In embodiments, the standard control is a median level of BMP2 obtained from a population of non-disease individuals. In embodiments, the standard control is a level of BMP2 obtained from non-disease tissue obtained from the patient. In embodiments, the standard control is a mean level of BMP2 obtained from a population of patients having a BMP2 and/or BMP2 associated disease (e.g., cancer). In embodiments, the standard control is a median level of BMP2 obtained from a population of patients having a BMP2 and/or BMP2 associated disease (e.g., cancer).

In embodiments, the pre-treatment level of secreted uPAR isoform2 in the pre-treatment biological sample is compared to a standard control. In embodiments, the pre-treatment level of secreted uPAR isoform2 is decreased relative to the standard control. In embodiments, the pre-treatment level of secreted uPAR isoform2 is increased relative to the standard control. In embodiments, the pre-treatment level of secreted uPAR isoform2 is essentially the same relative to the standard control. In embodiments, the standard control is a median level of secreted uPAR isoform2 obtained from a population of non-disease individuals. In embodiments, the standard control is a level of secreted uPAR isoform2 obtained from non-disease tissue obtained from the patient. In embodiments, the standard control is a mean level of secreted uPAR isoform2 obtained from a population of patients having a secreted uPAR isoform2 and/or secreted uPAR isoform2 associated disease (e.g., cancer). In embodiments, the standard control is a median level of secreted uPAR isoform2 obtained from a population of patients having a secreted uPAR isoform2 and/or secreted uPAR isoform2 associated disease (e.g., cancer).

In embodiments, administering the effective amount of MDA-7 reverses a multidrug chemoresistance. As used herein, “multidrug chemoresistance” refers to the mechanism by which cancers develop resistance to multiple chemotherapy drugs, resulting in the failure of chemotherapy drugs to induce cancer cell death, thereby leading to the expansion of drug resistant tumors.

In embodiments, the method further includes administering to the subject an additional anti-cancer agent. In embodiments, the additional anti-cancer agent is not MDA-7. In embodiments, the cancer patient has been further treated with an additional anti-cancer agent. In embodiments, the additional anti-cancer agent is a ROS inducer. Non-limiting examples of in clinic ROS inducers include arsenic trioxide, hydrogen peroxide, or pyocyanin. Thus, in embodiments, the ROS inducer is arsenic trioxide, hydrogen peroxide, or pyocyanin. In embodiments, the ROS inducer is arsenic trioxide. In embodiments, the ROS inducer is hydrogen peroxide. In embodiments, the ROS inducer is pyocyanin.

As described above, the ROS inducer may be delivered at a low dose. Thus, in embodiments, a low dose is about 0.01 μM, 0.05 μM, 0.1 μM, 0.2 μM, 0.3 μM, 0.4 μM, 0.5 μM, 0.6 μM, 0.7 μM, 0.8 μM, 0.9 μM, 1 μM, 5 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 200 μM, 300 μM, 400 μM, 500 μM, 600 μM, 700 μM, 800 μM, 900 μM, 1 mM.

It is also contemplated that downregulating miR-221, thereby upregulating beclin-1, via MDA-7 treatment is useful for treating cancers not expressing beclin-1 and not expressing MDA-7. Thus, in an aspect is provided a method of treating cancer in a subject in need thereof, wherein the subject has a cancer not expressing beclin-1 and not expressing MDA-7, the method including administering to the subject an effective amount of MDA-7.

In embodiments, the cancer expresses miR-221.

In embodiments, the method further includes, prior to administering the effective amount of MDA-7: (i) obtaining a pre-treatment biological sample from the subject; and (ii) detecting a pre-treatment level of beclin-1 in the pre-treatment biological sample.

In embodiments, the pre-treatment biological sample is a tumor biopsy. In embodiments, the pre-treatment biological sample is a blood sample. In embodiments, the pre-treatment biological sample includes a circulating tumor cell. In embodiments, the pre-treatment biological sample is a circulating tumor cell. In embodiments, the detecting includes performing real-time PCR. In embodiments, the detecting includes performing Western blotting analysis.

In embodiments, the pre-treatment level of beclin-1 in the pre-treatment biological sample is compared to a standard control. In embodiments, the pre-treatment level of beclin-1 is decreased relative to the standard control. In embodiments, the standard control is a level of beclin-1 obtained from a non-disease individual. In embodiments, the standard control is a mean level of beclin-1 obtained from a population of non-disease individuals. In embodiments, the standard control is a median level of beclin-1 obtained from a population of non-disease individuals. In embodiments, the standard control is a level of beclin-1 obtained from non-disease tissue obtained from the patient. In embodiments, the standard control is a mean level of beclin-1 obtained from a population of patients having a miR-221 and/or beclin-1 associated disease (e.g., cancer). In embodiments, the standard control is a median level of beclin-1 obtained from a population of patients having a miR-221 and/or beclin-1 associated disease (e.g., cancer).

In embodiments, the detecting a pre-treatment level of beclin-1 includes detecting a pre-treatment level of MMP, a pre-treatment level of TIMP3, a pre-treatment level of BMP2, a pre-treatment level of secreted uPAR isoform2 or a combination thereof in the pre-treatment biological sample. In embodiments, the detecting a pre-treatment level of beclin-1 includes detecting a pre-treatment level of MMP, a pre-treatment level of TIMP3, a pre-treatment level of

BMP2 and a pre-treatment level of secreted uPAR isoform2 in the pre-treatment biological sample. In embodiments, the detecting a pre-treatment level of beclin-1 includes detecting a pre-treatment level of MMP in the pre-treatment biological sample. In embodiments, the detecting a pre-treatment level of beclin-1 includes detecting a pre-treatment level of TIMP3 in the pre-treatment biological sample. In embodiments, the detecting a pre-treatment level of beclin-1 includes detecting a pre-treatment level of BMP2 in the pre-treatment biological sample. In embodiments, the detecting a pre-treatment level of beclin-1 includes detecting a pre-treatment level of secreted uPAR isoform2 in the pre-treatment biological sample. In embodiments, the detecting a pre-treatment level of beclin-1 includes detecting a pre-treatment level of beclin-1, a pre-treatment level of MMP, a pre-treatment level of TIMP3, a pre-treatment level of BMP2, a pre-treatment level of secreted uPAR isoform2 or a combination thereof in the pre-treatment biological sample. In embodiments, the detecting a pre-treatment level of beclin-1 includes detecting a pre-treatment level of beclin-1, a pre-treatment level of MMP, a pre-treatment level of TIMP3, a pre-treatment level of BMP2, and a pre-treatment level of secreted uPAR isoform2 in the pre-treatment biological sample.

Any of the embodiments of the methods described above are useful and applicable and therefore contemplated for the methods described in this section. Thus, as described for the methods above, the pre-treatment level of MMP, BMP2 or secreted uPAR isoform2 in the pre-treatment biological sample may be compared to a standard control. In embodiments, administering the effective amount of MDA-7 reverses a multidrug chemoresistance.

Similar to the methods described above, in embodiments, the method further includes administering to the subject an additional anti-cancer agent. The additional anti-cancer agent is not MDA-7. In embodiments, the cancer patient has been further treated with an additional anti-cancer agent. In embodiments, the additional anti-cancer agent is a ROS inducer as described herein (e.g., arsenic trioxide, hydrogen peroxide, or pyocyanin).

In embodiments, the MDA-7 treatment and the additional anti-cancer agent are administered in a combined synergistic amount.

In embodiments, the cancer patient has melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer. In embodiments, the cancer patient has melanoma. In embodiments, the cancer patient has prostate cancer. In embodiments, the cancer patient has neuroblastoma. In embodiments, the cancer patient has osteosarcoma. In embodiments, the cancer patient has renal carcinoma. In embodiments, the cancer patient has leukemia. In embodiments, the cancer patient has epithelial cancer. In embodiments, the cancer patient has pancreatic cancer. In embodiments, the cancer patient has glioblastoma. In embodiments, the cancer patient has thyroid papillary carcinoma. In embodiments, the cancer patient has esophageal squamous cell carcinoma. In embodiments, the cancer patient has breast cancer. In embodiments, the cancer patient has hepatocellular carcinoma. In embodiments, the cancer patient has liver cancer. In embodiments, the cancer patient has lung cancer. In embodiments, the cancer is a metastatic cancer.

It is further that the methods described herein may be effective in preventing or reducing cancer-associated angiogenesis. Therefore, in an aspect is provided a method of inhibiting cancer-associated angiogenesis in a subject in need thereof, the method including administering to the subject an effective amount of MDA-7.

IV. Methods of Additional Diseases

It is further contemplated that the method described herein, including embodiments thereof, are useful for treating diseases associated with aberrant miR-221 levels and/or activity.

In an aspect is provided a method of treating an autoimmune disease in a subject in need thereof, the method including administering to the subject an effective amount of MDA-7. In embodiments, the autoimmune disease is rheumatoid arthritis.

In an aspect is provided a method of treating an infectious disease in a subject in need thereof, the method including administering to the subject an effective amount of MDA-7. In embodiments, the infectious disease is tuberculosis. In embodiments, the infectious disease is influenza.

In an aspect is provided a method of treating an inflammatory disease in a subject in need thereof, the method including administering to the subject an effective amount of MDA-7. In embodiments, the inflammatory disease is psoriasis. In embodiments, the inflammatory disease is inflammatory bowel disease.

In an aspect is provided a treating a cardiovascular disease in a subject in need thereof, the method including administering to the subject an effective amount of MDA-7. In embodiments, the cardiovascular disease includes vascular calcification. In embodiments, the cardiovascular disease is premature coronary artery disease. In embodiments, the cardiovascular disease is subclinical atherosclerosis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

### mda-7/IL-24 Mediates Cancer Cell-Specific Death through Regulation of miR-221 and the beclin-1 Axis

Melanoma differentiation associated gene-7/Interleukin-24 (mda-7/IL-24) displays broad-spectrum anti-cancer activity in vitro, in vivo in preclinical animal models and in a phase I/II clinical trial in patients with advanced cancers, without harming normal cells or tissues. We presently demonstrate that mda-7/IL-24 regulates a specific subset of miRNAs, including cancer-associated miR-221. Both ectopic expression of mda-7/IL-24 or treatment with recombinant His-MDA-7 protein down regulate miR-221, while up regulating p27 and PUMA, in a panel of cancer cells culminating in cell death. Mda-7/IL-24-induced cancer cell death was dependent on reactive oxygen species induction and was rescued by over expressing miR-221. Beclin-1 was identified as a new transcriptional target of miR-221 and mda-7/IL-24 regulated autophagy through a miR-221/beclin-1 feedback loop. In a human breast cancer xenograft model, miR-221 overexpressing MDA-MB-231 clones were more aggressive and resistant to mda-7/IL-24-mediated cell death than MDA-MB-231 parental clones. This is the first demonstration that mda-7/IL-24 directly regulates microRNA expression in cancer cells and highlights the importance of a novel mda-7/IL-24-miR-221-beclin-1 loop in mediating cancer cell-specific death by this therapeutic IL-10 gene-family member cytokine.

mda-7/IL-24 (1) has potent anti-tumor activity in almost all types of cancers (2-6). mda-7/IL-24 is a member of the IL-10-related cytokine gene family, which was cloned using subtraction hybridization and induction of terminal cancer cell differentiation in melanoma cells (1). Extensive research has confirmed the ubiquitous anti-tumor properties of mda-7/IL-24 both in in vitro cell cultures and animal models (6). mda-7/IL-24 displayed safety and efficacy in a Phase I/II clinical trial in patients with several advanced cancers (7, 8). Forced over expression of mda-7/IL-24 inhibits angiogenesis (9, 10), sensitizes cancer cells to radiation or chemotherapy (3-6) and elicits potent ‘bystander’ antitumor activity (11). Physical interaction of MDA-7/IL-24 protein with the chaperone protein BiP/GRP78 initiates an unfolded protein response (UPR) in cancer cells that leads to apoptosis (12). Gene expression studies have illustrated a number of apoptotic and cell cycle molecules regulated by mda-7/IL-24 (13).

MicroRNAs are small noncoding RNAs, which degrade RNAs, negatively affect the stability of RNAs or block the translation of mRNAs (14-16). MicroRNAs are aberrantly expressed in many diseases including cancer (17, 18). MicroRNA-221 is an important regulator, whose up regulation has been described in several types of cancers and several reports suggest that miR-221 can be used as a therapeutic target for cancer. Many tumor suppressors have been reported to be targets of miR-221. miR-221 regulates cell cycle through p27 (19) and apoptosis through PUMA (20). Additionally, by targeting the estrogen receptor (ER) it blocks the action of tamoxifen and hence targeting miR-221 can promote susceptibility to tamoxifen-mediated cell death in ER positive breast cancers (21). Other tumor suppressor targets of miR-221 include PTEN (22), p57 (23), FOXO3A (24), and TIMP3 (22). By regulating these targets miR-221 plays a critical role in cancer progression. Felicetti et al. reported that the promyelocytic leukemia zinc finger (PLZF) transcription factor functions as a transcriptional repressor of miR-221 (25), however, the mechanism(s) regulating miR-221 requires further elucidation.

Beclin-1, the mammalian homologue of Atg6 of yeast is a promoter of autophagy. Expression of beclin-1 is altered in different disease states including cancer. In several types of cancer aberrant mRNA/protein expression of beclin-1 has been observed (26). The underlying mechanism of this altered expression of beclin-1 is largely unknown. In the present study, we document crosstalk between tumor suppressors and oncogenes, i.e., mda-7/IL-24, miR-221, and beclin-1. Ad.mda-7 infection down regulates miR-221, which in turn up regulates beclin-1 and promotes toxic autophagy that switches to apoptosis. Our findings suggest that miR-221 is a downstream participant in mda-7/IL-24-mediated cell death and cells overexpressing miR-221 are resistant to mda-7/IL-24-mediated cell death. Finally, we show that ROS plays a key role in this pathway and a novel mda-7/IL-24-miR-221-beclin-1 axis is critical in mda-7/IL-24-mediated cell death.

Materials and Methods

Plasmids, cell lines and stable clones. The miR-221 and anti-miR-221 constructs were from GeneCopoeia (Rockville, Md.). Beclin-1 3′UTR construct was from Origene (Rockville, Md.). Beclin-1 construct was from Addgene (Cambridge, Mass.). The Beclin-1-UTR mutant was cloned from the wild type Beclin-1-UTR by standard site-directed mutagenesis (27). Cell lines used in this study included DU-145, MCF-7, T-47D, MDA-MB-231, ZR-751, SK-BR-3, RPMI-7951, NB-1691, SK-N-SH, IM-PHFA, RWPE-1, HMEC, and A549. These cells were obtained from the American type culture collection (ATCC) (Manassas, VA), with the exception of IM-PHFA, which was established in our laboratory (28), and were maintained as described by the ATCC. ATCC authenticates these cell lines using short tandem repeat (STR) analysis. All the cell lines were expanded and frozen immediately after receipt. The cumulative culture length of the cells was less than 6 months after recovery. Early passage cells were used for all experiments. Human mammary epithelial cells (HMEC) were purchased from Lonza, Basel, Switzerland. NB-1691 cells were a kind gift from Dr. Alan Houghton from St Jude children's research hospital (Memphis, Tenn.). All the cell lines were frequently tested for mycoplasma contamination using a mycoplasma detection kit from Sigma. Stable clones expressing miR-221 and beclin-1 were established in MDA-MB-231 cells as described previously (28).

Western blotting analysis. Western blotting was done as described (29). The primary antibodies used in this study were MDA-7/IL-24 (Genhunter Corporation, Nashville, Tenn.), EF1α (Upstate biotechnology, Lake Placid, N.Y.), p27 and PUMA (Cell Signaling Technology, Danvers, Mass.), and beclin-1 (Abcam, Cambridge, Mass.). Secondary antibodies used in this study were from Sigma, (St. Louis, Mo.).

Real-time PCR. Total RNA and microRNA-enriched fractions of RNA were isolated from cells using the RNA and microRNA isolation kits, respectively, from Qiagen (Hilden, Germany) Real time PCR was performed with the taqman master mix and probe were from Applied Biosystems, Foster City, CA. Data were analyzed by Graphpad prism software.

Transient transfection and reporter gene assay. Transient transfection used the lipofectamine reagent from Invitrogen, Carlsbad, Calif. For luciferase assay, cells were transfected with the 3′UTR construct of beclin-1 with or without miR-221 with the pRLTK luc construct encoding renilla luciferase control. Cells were incubated for 24 hours and then luciferase assays were done using the dual-luciferase assay kit from Promega, Madison, Wis..

Cell proliferation assay. Cell proliferation was measured by standard MTT (3-(4, 5-di methyl thiazol-2-yl)-2,5 diphenyl tetrazolium bromide) assay as described earlier (11). Colony formation assays were done as described previously (29).

Tumor xenograft studies. Tumor xenografts were established subcutaneously in both flanks of 6-week old female athymic mice (Charles River Laboratories, Wilmington, Mass.) by injecting 0.5×106 MDA-MB-231 or MDA-MB-231 cells overexpressing miR-221 or beclin-1 mixed with Matrigel in a 1:1 ratio. Once tumors reached a measurable size of approximately 100 mm3, the mice were divided into different groups and treated as described in the figure and figure legend. When the tumors in the control group reached the maximum allowable limit, mice were sacrificed and tumor weight was measured. Tumor size was also measured and plotted. Animals were maintained under the guidelines of the National Institute of Health and under evaluation and approval of the Institutional Animal Care and Use Committee (Virginia Commonwealth University). Food and water were provided ad libitum.

Reactive oxygen species (ROS) measurement. The amount of reactive oxygen species that is produced was quantified by staining cells with carboxy-2′, 7′-dichloro dihydro fluorescein diacetate (Life technologies, Molecular probes, Grand Island, N.Y.) in 1× phosphate buffered saline. Fluorescence was measured using a green filter after 30 minutes. Experimental conditions are described in the figures and figure legends.

Live-dead assay. The number of live and dead cells was observed by confocal laser microscope (Zeiss, Germany) after staining with live/dead staining reagent (Invitrogen, Carlsbad, Calif.) as per the manufacturer's protocol. The images were analyzed by Zeiss software.

Apoptosis assay. MDA-MB-231 cells were treated as indicated in the figure. After 72 hours, cells were analyzed for apoptosis using the Annexin-V-FITC/propidium iodide apoptosis detection kit (BD Biosciences, San Jose, Calif.) and subjected to flow cytometry analysis using BDFACS Cantoll and BDFACS DIVA software (BD Biosciences, San Jose, Calif.).

Autophagy assay. The cellular acidic compartment was observed as a marker of autophagy and quantified by staining with acridine orange as described previously (30). Briefly, cells were stained with 1 μg/ml acridine orange (Sigma, St. Louis, Mo.) for 10 minutes. Cells were washed with 1× PBS and then the numbers of cells, which have increased acidic vacuoles, were measured using flow cytometry (BDFACS Cantoll) and analyzed with BDFACS DIVA software. Approximately, 10000-gated cells were analyzed.

Statistical analysis. The data are presented as the mean +/−S.D. of the values from 3 to 5 independent experiments and statistical analysis was performed using either student's t-test or one-way anova. P-value<0.05 was considered to be significant. This was done using the graph pad prism software.

Results

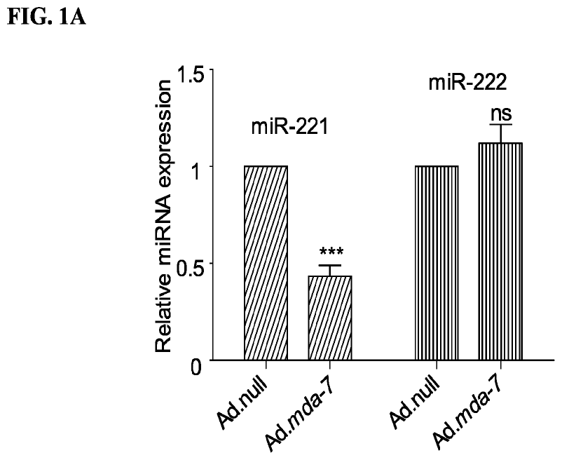

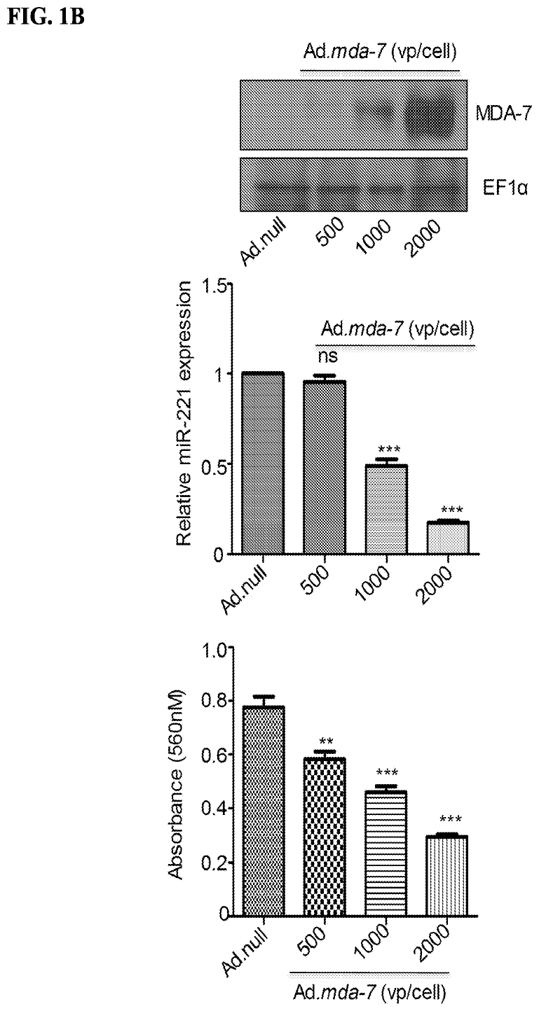

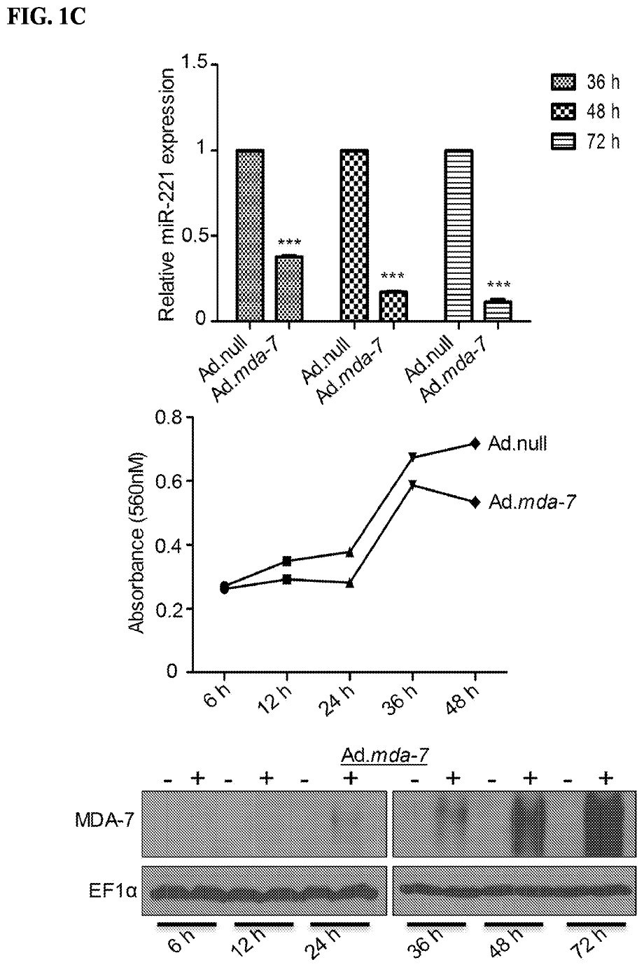

MDA-7 regulates miR-221. mda-7/IL-24 is recognized for its specific and selective tumor cell-killing effects without harming normal cells. To examine the role of mda-7/IL-24 on the regulation of different microRNAs that are potentially involved in cell death or apoptosis, we overexpressed mda-7/IL-24 using a viral vector expressing mda-7/IL-24 (Ad.mda-7) in MDA-MB-231 cells, an aggressive triple negative breast cancer cell line. The microRNA-enriched fraction was isolated and real time PCR was done for a series of microRNAs related to cell death/apoptosis. A number of microRNAs including miR-200c, let7c, and miR-320 were found to be deregulated after treatment with mda-7/IL-24 (FIG. 8). miR-200c which regulates tumor metastasis and epithelial-mesenchymal transition, was found to be down regulated by mda-7/IL-24. Another microRNA, miR-17, reported in G1-S transition of cell cycle, was found to be up regulated by mda-7/IL-24. microRNA-185, a tumor suppressor, reported in many cancers was found to be up regulated in mda-7/IL-24-infected cells. The members of let-7 microRNA family were also deregulated in mda-7/IL-24-infected cells. While let-7e showed no change, let-7c was up regulated in mda-7/IL-24-infected cells. Among these miRNAs, miR-221 is one of the microRNAs reported in a number of cancers and it exhibits an expansive role in different pathways deregulated in cancer. miR-221 targets p27, a key modulator of cell cycle (19) and PUMA, a proapoptotic gene that is degraded by miR-221 (20). PTEN, a potent tumor suppressor is also down regulated by miR-221 (22). As shown in FIG. 1A, we found miR-221 was down regulated in mda-7/IL-24-treated MDA-MB-231 cells, while no alteration was observed in the level of miR-222. mda-7/IL-24-mediated down regulation of miR-221 occurred in a dose-dependent manner, which correlated with exogenous protein expression (MDA-7/IL-24) and inhibition of cell growth (FIG. 1B). The down regulation of miR-221 by mda-7/IL-24 also occurred in a temporal manner in a time point kinetic study (FIG. 1C).

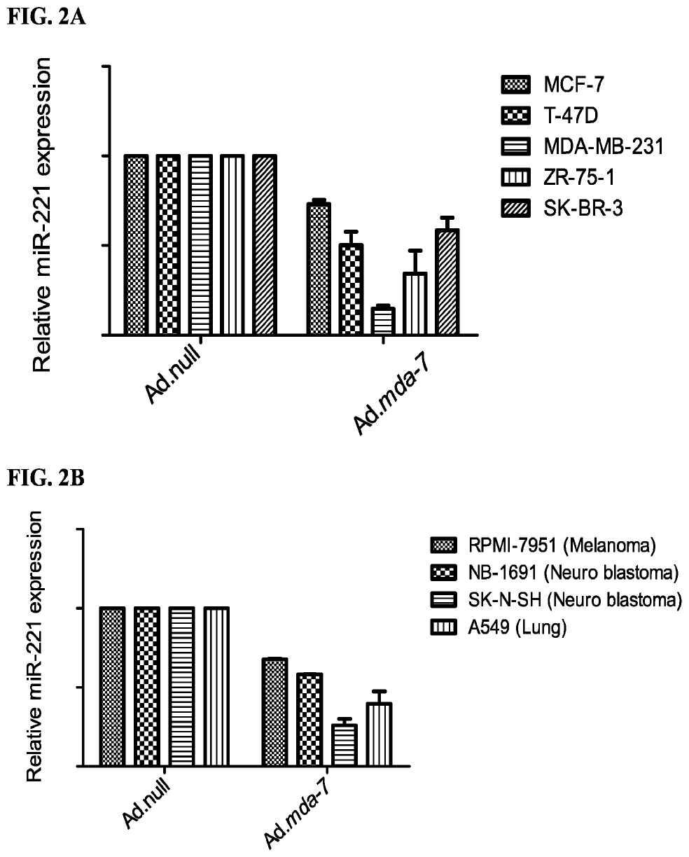

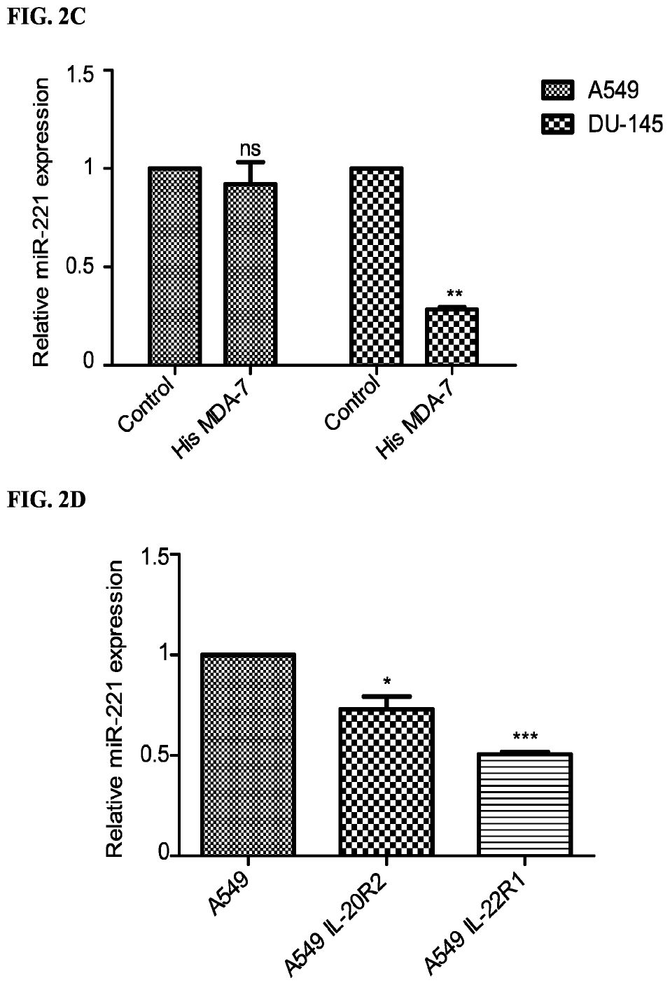

mda-7/IL-24 down regulates miR-221 in diverse cancer cell lines. Breast cancer is classified on the basis of hormone receptor expression [estrogen receptor (ER) and progesterone receptor (PR)] and also HER2/Neu status. Triple negative breast cancers express higher levels of miR-221 than ER/PR/HER2 positive breast cancers (31). Our initial observation confirmed that miR-221 was down regulated by mda-7/IL-24 in MDA-MB-231 cells, a triple negative breast cancer cell line. Next we checked regulation of miR-221 after mda-7/IL-24 expression in a panel of breast cancer cell lines with variable ER/PR/HER2 status. We infected MCF-7, T-47D, ZR-75-1 (triple positive) and SK-BR-3 (ER −ve, PR −ve, HER2 +ve) with Ad.null and Ad.mda-7 and collected miRNA enriched fractions and checked the level of miR-221. Interestingly, we found that miR-221 levels decreased with mda-7/IL-24 over expression irrespective of the breast cancer cells receptor status (FIG. 2A). Additionally, we assayed other cancer cell lines, i.e., melanoma, neuroblastoma, and lung cancer, and found a similar downregulation of miR-221 following overexpression of mda-7/IL-24 (FIG. 2B). This endorses the hypothesis that miR-221 may be a potential target for mda-7/IL-24 in a diverse array of cancers and suggests a new pathway of mda-7/IL-24-mediated gene regulation.

As a cytokine and a member of the IL-10 cytokine gene family, MDA-7/IL-24 signals through receptor dimers consisting of an R1 type receptor and an R2 type receptor (IL-20R1 and IL-20R2; IL-22R1 and IL-20R2; or a unique receptor pair IL-20R1 and IL-22R1) in order to activate downstream signaling events (5, 6). We used purified recombinant MDA-7/IL-24 protein (11) to confirm further the regulation of miR-221 by MDA-7/IL-24. We treated A549 cells (lung cancer cells which lack a full set of R1 and R2, IL-20/IL-22, receptors) and DU-145 cells (prostate cancer cells containing both receptor types) with His tagged MDA-7 and measured the level of miR-221. miR-221 expression decreased in DU-145 cells following treatment with His-MDA-7, while the level remained unchanged in A549 cells, which lacks the cognate receptor pairs (FIG. 2C). Overexpression of the IL-20R2 or IL-22R1 receptors in A549 cells rendered these cells sensitive to miR-221 down regulation after treatment with MDA-7/IL-24 recombinant protein (FIG. 2D).

To determine if the ability of mda-7/IL-24 to regulate miR-221 was a general phenomenon in both cancer and normal cells we also checked the level of miR-221 following Ad.mda-7 infection in a series of normal immortal human cell lines (IM-PHFA, RWPE-1 and HMEC). No substantial changes in miR-221 levels were evident in any of these normal cells following infection with Ad.mda-7 further supporting the cancer specificity of this cytokine (FIG. 9).

Over expression of miR-221 rescues cells from mda-7/IL-24-mediated cell death. To study the effect of miR-221 on mda-7/IL-24-mediated cell death, MDA-MB-231 cells were transfected with a pCDNA3.1 or miR-221 vector and infected with Ad.null or Ad.mda-7 (2000vp/cells). After 72 hours cells were analyzed for cell death using the Annexin-V binding assay and flow cytometry. As shown in FIG. 3A, MDA-MB-231 cells showed increased apoptosis following infection with Ad.mda-7 vs. Ad.null. Cells that overexpress miR-221 had significantly less cell death suggesting a protective role of miR-221 in mda-7/IL-24-induced apoptotic death. This observation was also supported using a cell proliferation assay. Cells were transfected with miR-221 and infected with Ad.null or Ad.mda-7 and cell proliferation was analyzed with a MTT assay at 72 hours post-infection. As predicted, overexpression of mda-7/IL-24 decreased cell growth, which was rescued in miR-221 overexpressing cells (FIG. 3B). A live dead staining assay further confirmed this data (FIG. 3C). Next, we generated stable cell lines overexpressing miR-221 in MDA-MB-231 cells (FIG. 3D) and did a clonogenic assay following infection with Ad.null or Ad.mda-7 in pCDNA3.1 control vector or miR-221 vector overexpressing stable clones. Infection with Ad.mda-7 resulted in a complete inhibition of colony formation in pCDNA3.1-transfected cells, however there were viable colonies observed in miR-221 overexpressing stable cell clones Cl. 1 and Cl. 4 (FIG. 3E). A direct correlation between enhanced colony formation and levels of miR-221 expression was evident when plating Ad.mda-7 infected cells at low density (50 as opposed to 2000 cells/6-cm plate) (data not shown).

mda-7/IL-24 regulation of miR-221 expression is ROS-dependent. Ectopic expression of MDA-7/IL-24 in glioblastoma multiforme cells increases thioredoxin and manganese super oxide dismutase (SOD2) levels, without altering SOD1 protein levels (32). MDA-7/IL-24-mediated cell killing relies on reactive oxygen species (ROS) generation, which is one of the key mediators of MDA-7/IL-24 toxicity in cancer cells (33). In these contexts, we determined if ROS inducers could enhance mda-7/IL-24 down regulation of miR-221. Infection of MDA-MB-231 breast cancer cells with 500 vp/cell of Ad.mda-7 did not significantly alter miR-221 levels. Similarly, treatment with low doses of ROS inducers, Arsenic trioxide (ATO, 1 μM), hydrogen peroxide (10 μM) or pyocyanin (50 μM) did not alter miR-221 levels in MDA-MB-231 cells. In contrast, a combination of Ad.mda-7 plus ATO (FIG. 4A), pyocyanin (FIG. 4B) or hydrogen peroxide (FIG. 4C) at the low doses indicated above significantly decreased the level of miR-221. These observations confirm a role of reactive oxygen species in mda-7/IL-24-mediated down regulation of miR-221. While ATO, hydrogen peroxide or pyocyanin down regulated the miR-221 level with Ad.mda-7, treatment with NAC, a known anti-oxidant abrogated down regulation of miR-221. This also supports earlier published studies, which have shown the role of ROS in mda-7/IL-24-mediated cell death (33). As shown in FIG. 10, elevated levels of ROS inducers decreased the level of miR-221 further supporting a ROS-mediated regulation of miR-221.

Beclin-1 is a direct target of miR-221. miR-221 inhibits autophagy induction, which leads to heart failure by deregulating the p27/CDK2/mTOR pathway (34). A regulatory link between miR-221 and Beclin-1 has been suggested, since Beclin-1 is regulated by HDAC6 (35) and HDAC6 is regulated by miR-221 (36). Additionally, a regulatory role of mda-7/IL-24 in toxic autophagy has been described (37). These observations prompted us to investigate the regulatory role of mda-7/IL-24-miR-221 axis in the autophagy process. First, we checked the expression pattern of Beclin-1 following overexpression of miR-221. MDA-MB-231 cells were transfected with a pCDNA3.1 vector or a miR-221 expressing construct. Overexpression of miR-221 resulted in beclin-1 down regulation (FIG. 5A) and increasing doses of miR-221 significantly down regulated beclin-1 at the transcript and protein level in a dose-dependent manner in MDA-MB-231 cells (FIGS. 5B, 5C and FIG. 11). To validate the role of miR-221 on the transcriptional regulation of beclin-1, we performed a luciferase reporter gene assay using a 3′ UTR beclin-1 construct that covers 600-bp downstream of the beclin-1 stop codon. The miR-221 transfected HeLa cells showed a significantly lower luciferase activity than the vector-transfected cells (FIG. 5D) suggesting that beclin-1 is a potential target of miR-221. The assay was validated with a mutated 3′ UTR construct of beclin-1. miR-221 failed to down regulate the mutant construct, which has no binding site for miR-221 (FIG. 5D). It was reported earlier that mda-7/IL-24 over expression led to enhanced beclin-1 expression (35). These data confirm that beclin-1 is a potential target of miR-221 and suggest a mechanism of mda-7/IL-24-mediated autophagy regulation through a miR-221 and beclin-1 pathway. To further confirm miR-221 regulation of beclin-1 we blocked miR-221 by a specific anti-miR-221 in MDA-MB-231 cells and measured the expression of beclin-1. The addition of anti-miR-221 prevented the degradation of beclin-1 in basal (FIG. 5E and FIG. 12) and Ad.mda-7-infected cells (FIG. 12). Expression of p27 and PUMA were also checked to validate the experimental controls (FIG. 12). The same trend was observed in two other cell lines derived from other cancers, i.e., lung (A549) and prostate (DU-145) and also in an additional breast cancer cell line, i.e. ZR-751 (FIG. 5C, FIGS. 11 and 12). These data confirm that beclin-1 is a potential target of miR-221.

To investigate further the potential regulatory axis of mda-7/IL-24-miR-221-beclin-1, we transfected cells with a miR-221 construct and infected cells with Ad.mda-7. As observed earlier, mda-7/IL-24 up regulated beclin-1 and simultaneous over expression of mda-7/IL-24 with miR-221 diminished the mda-7/IL-24-mediated enhanced expression of beclin-1 (FIG. 5C). Additionally, we evaluated autophagy induction using an acridine orange-based staining method. As shown in FIG. 5F, ectopic expression of miR-221 diminished autophagy induction and overexpression of beclin-1 or mda-7/IL-24 rescued the autophagy process in these cells confirming further a role of the mda-7/IL-24-miR-221-beclin-1 regulatory loop in autophagy induction.

Rapamycin, another autophagy inducer, up regulates beclin-1 (38). To study the role of Rapamycin and miR-221 on beclin-1 levels, different cancer cell lines were transfected with miR-221 and treated with Rapamycin. While Rapamycin up regulated beclin-1 protein levels, ectopic expression of miR-221 decreased this upregulation (FIG. 13). This result confirms miR-221-mediated beclin-1 regulation and also explains yet another mechanism showing miR-221 can deregulate Rapamycin-induced autophagy.

mda-7/IL-24 regulates miR-221 expression in vivo. To investigate the role of mda-7/IL-24 on miR-221 in vivo, MDA-MB-231 cells over expressing miR-221 or miR-221 plus beclin-1 were injected subcutaneously to establish tumor xenografts in female athymic nude mice. After a palpable tumor (100 mm3) developed in approximately 10 days, the tumors were injected with 8 intramural injections over a 3-week period with 1×108 viral particles of Ad.null or Ad.mda-7. In control vector-transfected cells a significant growth inhibitory effect was evident, but in miR-221 over expressing cells the effect of Ad.mda-7 was less apparent both in the injected left tumor, and in the uninjected right tumor, as previously observed when infecting these cells in vitro (FIG. 6A and B). Interestingly, overexpressing beclin-1 in miR-221-transfected cells sensitized these cells to mda-7/IL-24-induced cell death. The expression of miR-221 was confirmed by RQ-PCR (FIG. 6C) and the expression of MDA-7/IL-24 and beclin-1 was validated and quantified in the tumor sections by immunohistochemistry (FIG. 6D, FIG. 14). A modest increase in p27 and PUMA expression was also evident in tumor sections from Ad.mda-7-treated tumors vs. Ad.null-treated tumors (FIG. 15). Taken together these results demonstrate the significance of miR-221 and beclin-1 in triggering mda-7/IL-24-mediated cell death in cancer cells.

Discussion

Reprogramming of cancerous cells to undergo toxic autophagy (39) or apoptosis (40) is a viable strategy for treatment of cancer. The discovery of mda-7/IL-24 using subtraction hybridization in melanoma has further advanced this opportunity (1, 2). The multiple distinctive functions of mda-7/IL-24 in cancer therapy include tumor-specific killing through combined effects of apoptosis and toxic autophagy, potent “bystander” anti-cancer activity, immunomodulation, inhibition of cell proliferation, and suppression of angiogenesis (3-6). We now demonstrate that miR-221, an oncogenic miRNA, is down regulated by over expression of mda-7/IL-24 in a cancer cell-specific manner Using a panel of breast, lung, prostate, and neuroblastoma cell lines we document a significant decrease in the level of miR-221 following adenoviral-mediated delivery of mda-7/IL-24. This down regulation of miR-221 correlates with mda-7/IL-24-mediated cell death and over expression of miR-221 blocks cell death induced by mda-7/IL-24. Production and secretion of MDA-7/IL-24 following treatment with purified recombinant cytokine or infection with Ad.mda-7 decreases cell growth and induces apoptosis in cancer cells, but not in normal cells. Additionally, secreted MDA-7/IL-24 also induces apoptosis in surrounding cells as well as distant tumor cells through “bystander” antitumor effects (28). Furthermore, MDA-7/IL-24 protein induces production of endogenous MDA-7/IL-24 through an autocrine/paracrine loop (11). Using recombinant His-MDA-7 we found that MDA-7/IL-24 also down regulates miR-221, uniquely in IL-20/IL-22 receptor positive cancer cells. Reconstruction of cognate receptors in receptor complex negative cells renders them vulnerable to His MDA-7 treatment and consequently results in down regulation of miR-221. These results further demonstrate the relevance of the MDA-7/IL-24-miR-221 axis in promoting cancer-specific cell death. In these contexts, miR-221 represents a novel downstream target of mda-7/IL-24 specific to cancer cells that mediates its biological anti-cancer functions both in vitro and in vivo.

The profound anticancer action of mda-7/IL-24 in cell culture and pre-clinical animal models led to its entry into the clinic and has culminated in a successful phase-I/II clinical trial (3, 4, 7, 8, 41). These observations reinforce the relevance of defining the mechanism of anti-cancer activity of mda-7/IL-24. Additionally, many combinatorial approaches have been shown to further enhance mda-7/IL-24's antitumor activities. Defining ways of making this therapeutic even better is of significant import and very relevant for the treatment of primary and advanced cancers.

Reactive oxygen species play a prominent role in mda-7/IL-24-restricted antitumor functions (33). Multiple cellular and physiological processes impacted on by mda-7/IL-24 are regulated by ROS. In the context of pancreatic cancer, where there is a ‘translational block’ of mda-7/IL-24 mRNA into protein, ROS can reverse this inhibition resulting in enhanced association of mda-7/IL-24 mRNA with polyribosomes and translation into protein thereby resulting in pancreatic cancer cell death (42-44). We now show that mda-7/IL-24-mediated down regulation of miR-221 is ROS-dependent and treatment with anti-oxidants can reverse this process. These results accentuate a path for the development of rational combinatorial approaches for the treatment of aggressive tumors by combining mda-7/IL-24 with other in-clinic ROS-inducing chemotherapeutic agents.

MicroRNAs (miRNAs) play a central role in regulating different normal and pathological pathways, including development and cancer, respectively. Different microRNAs instigate diverse effects in a cell and tissue context-dependent manner depending on the target gene they regulate (45). Prior studies indicate that miR-221 is significantly upregulated in different cancers (17). To identify potential new targets of miR-221 we investigated the expression of some of the major proteins involved in autophagy, apoptosis and cell cycle. The regulation of beclin-1 expression by miR-221 was demonstrated by transfection of tumor cells with a miR-221 mimic, which resulted in a decrease in beclin-1 expression at both the mRNA and protein level. This relationship was confirmed further by transfection with anti-miR-221, which resulted in up regulation of beclin-1 expression. Beclin- 1-mediated protective/toxic autophagy, depending on cellular context, plays a decisive role in cell survival/death and aberrant expression of beclin-1 has been reported in different diseases including cancer (26, 46-49). Rapamycin is a well described autophagy inducer (50) and it also up regulates beclin-1 (36). Our results confirm that miR-221 not only degrades basal but also Rapamycin- and mda-7/IL-24-induced beclin-1 expression. Although it has been shown that mda-7/IL-24 promotes toxic autophagy, the detailed mechanism is not well understood. We now show that beclin-1, a key player in autophagy, is a new target of miR-221 and mda-7/IL-24 can promote toxic autophagy through a miR-221/beclin-1 axis. In addition to other regulators, i.e., p27, BAX, GADDs, Stat3, p38MAPKs, Bc12/Bc1-xL and PUMA, Beclin-1 is a new target of mda-7/IL-24 (summarized in FIG. 7). This study of beclin-1 regulation by miR-221 and miR-221 regulation by mda-7/IL-24 warrants further investigation and these studies have potential to yield new insights into the regulation of autophagy and the association of this phenomenon with various disease states.

### Example 2

### Novel Mechanism of mda-7/IL-24-Induction of Cancer Cell Specific Death

Melanoma differentiation associated gene-7/Interleukin-24 (mda-7/IL-24) displays broad spectrum anti-cancer activity in vitro, in vivo in preclinical animal models and in a phase I/II clinical trial in patients with advanced cancers, without harming normal cells or tissues. We demonstrate here that mda-7/IL-24 regulates a specific subset of miRNAs, including cancer associated miR-221. Both ectopic expression of mda-7/IL-24 or treatment with recombinant His-MDA-7 protein down regulate miR-221, while up regulating p27 and PUMA, in a panel of cancer cells culminating in cell death. Mda-7/IL-24-induced cancer cell death was dependent on reactive oxygen species induction and was rescued by over expressing miR-221. Beclin-1 was identified as a new transcriptional target of miR-221 and mda-7/IL-24 regulated autophagy through a miR-221/beclin-1 feedback loop. In a human breast cancer xenograft model, miR-221 overexpressing MDA-MB-231 clones were more aggressive and resistant to mda-7/IL-24-mediated cell death than MDA-MB-231 parental clones.

This is the first demonstration that mda-7/IL-24 directly regulates microRNA expression in cancer cells and highlights the importance of a novel mda-7/IL-24-miR-221-beclin-1 loop in mediating cancer cell-specific death by this therapeutic IL-10 gene-family member cytokine.

Considering the pivotal role of miR-221 in determining tumor aggressiveness and survival, directly targeting miR-221 expression by mda-7/IL-24 would be useful in developing targeted therapies for this important component of cancer pathogenesis.

Current studies state that mda-7/IL-24 can be used as a therapeutic cytokine. We show that mda-7/IL-24-mediated downregulation of miR-221 is ROS-dependent and treatment with anti-oxidants can reverse this process. Use of ROS inducers can increase its efficacy in different cancers. These results accentuate a path for the development of rational combinatorial approaches for the treatment of aggressive tumors by combining mda-7/IL-24 with other in clinic ROS-inducing chemotherapeutic agents.

The mechanism by which ROS inducers regulate miR-221 expression remains to be deciphered. We are working on this pathway to have a detailed knowledge of the system. This could lead to new combinatorial approaches to selectively target cancers for cell death.

The survival rate of a cancer patients with specific advanced cancers is dismal despite current progress in cancer therapies. This may be the result of chemo resistance or therapy resistance resulting in poor patient prognosis and relapse. MDA-7/IL-24 is a protein that is known to act as a tumor suppressor in several cancers, and its role in negatively regulating cancer cell survival is experimentally suggested and our results show that MDA-7/IL-24 can be used as a therapy for multiple diverse cancers.

### Example 3

### mda-7/IL-24 in Human Diseases

Subtraction hybridization identified genes displaying differential expression as metastatic human melanoma cells terminally differentiated and lost tumorigenic properties by treatment with recombinant fibroblast interferon and mezerein. This approach permitted cloning of multiple genes displaying enhanced expression when melanoma cells terminally differentiated, called melanoma differentiation associated (mda) genes. One mda gene, mda-7, has risen to the top of the list based on its relevance to cancer and now inflammation and other pathological states, which based on presence of a secretory sequence, chromosomal location and an IL-10 signature motif has been named interleukin-24 (MDA-7/IL-24). Discovered in the early 1990's, MDA-7/IL-24 has proven to be a potent, near ubiquitous cancer suppressor gene capable of inducing cancer cell death through apoptosis and toxic autophagy in cancer cells in vitro and in pre-clinical animal models in vivo. In addition, MDA-7/IL-24 embodied profound anti-cancer activity in a Phase I/II clinical trial following direct injection with an adenovirus (Ad.mda-7; INGN-241) in tumors in patients with advanced cancers. In multiple independent studies, MDA-7/IL-24 has been implicated in many pathological states involving inflammation and may play a role in inflammatory bowel disease, psoriasis, cardiovascular disease, rheumatoid arthritis, tuberculosis and viral infection. This review provides an up-to-date review on the multifunctional gene mda-7/IL-24, which may hold potential for the therapy of not only cancer, but also other pathological states.

Melanoma differentiation associated gene-7 (MDA-7), also known as interleukin-24 (IL-24), is a secreted cytokine and a member of the IL-10 gene family Although MDA-7/IL-24 was discovered several decades ago, new discoveries of the role that MDA-7/IL-24 plays in normal physiology as well as in multiple human pathologies are still unfolding. So far, researchers have confirmed that MDA-7/IL-24 is not only involved in normal immune function and wound healing, but it also has several additional beneficial effects in a variety of human diseases. As examples, MDA-7/IL-24 functions as an anti-cancer gene in multiple diverse cancers including melanoma (Sarkar et al., 2008), prostate cancer (Greco et al., 2010; Lebedeva et al. 2003a; 2003b), breast cancer (Bhutia et al., 2013; Menezes et al., 2015; Pradhan et al., 2013; Sarkar et al., 2005), osteosarcoma (Zhuo et al., 2017), neuroblastoma (Bhoopathi et al., 2016), pancreatic cancer (Sarkar et al., 2015), renal carcinoma (Park et al., 2009), leukemia (Rahmani et al., 2010), lung cancer (Lv et al., 2016; Shapiro et al., 2017), esophageal squamous cell carcinoma (Ma et al., 2016a), and hepatocellular carcinoma (Wang et al., 2007). MDA-7/IL-24 provides protection against autoimmune diseases and bacterial infections (Leng et al., 2011; Ma et al., 2009). MDA-7/IL-24 is also relevant in inflammation (Pasparakis et al., 2014), rheumatoid arthritis (Kragstrup et al., 2008) and cardiovascular diseases (Vargas-Alarcon et al., 2014). In this review, we discuss in detail the roles of MDA-7/IL-24 in both normal physiology as well as the various disease states mentioned above. We begin with a discussion of the characteristic features of MDA-7/IL-24 that allows this molecule to play a key role in normal cellular function as well as contributing to a variety of disease states.

CHARACTERISTIC FEATURES OF MDA-7/IL-24. We begin with an overview of the initial cloning of the MDA-7/IL-24 gene, followed by its structure, isoforms, and modifications that have helped enhance MDA-7/IL-24 potency. We then discuss the receptors that MDA-7/IL-24 utilizes for cellular signaling.

IDENTIFICATION OF MDA-7/IL-24. As the name suggests, MDA-7 was initially identified and cloned from terminally differentiating human melanoma cells in the Fisher laboratory by Jiang in 1993 and reported in detail in 1995 (Jiang and Fisher, 1993; Jiang et al., 1995). HO-1 human metastatic melanoma cells were treated with a combination of recombinant human fibroblast interferon (IFN-beta) and mezerein (MEZ) to induce terminal differentiation and suppression of growth and tumorigenic abilities. Next subtraction hybridization of cDNA libraries was performed to assess genes that were differentially expressed in melanoma cells before and after terminal differentiation (Jiang and Fisher, 1993). MDA-7 was identified as one of the transcripts that was induced in terminally differentiating melanoma cells (Jiang and Fisher, 1993; Jiang et al., 1995). In subsequent years, MDA-7 was found to have tumor suppressive abilities against several different cancer indications, while leaving normal counterparts unharmed (Jiang et al., 1996; Su et al., 1998). In 2001, Huang and colleagues in the Fisher laboratory identified the genomic structure and chromosomal localization of MDA-7 (Huang et al., 2001). They determined that MDA-7 was located in a region of the chromosome that contained a cluster of genes associated with the IL-10 cytokine family (Huang et al., 2001). MDA-7 also had an IL-10 signature sequence and was specifically expressed in tissues associated with the immune system including the spleen, thymus and peripheral blood leukocytes (Huang et al., 2001). Given the conserved chromosomal location, presence of a putative secretory motif, an IL-10 signature sequence and the expression profile of MDA-7, the Human Gene Organisation (HUGO) designated this gene as interleukin-24 (IL-24) (Sarkar et al., 2002a). Additionally in 2002, Caudell and colleagues provided evidence that MDA-7/IL-24 had functional immunostimulatory attributes justifying its designation as an interleukin (Caudell et al., 2002).

STRUCTURE OF MDA-7/IL-24. Located on chromosome 1q32-33 in humans, MDA-7/IL-24 is a secreted cytokine that belongs to the IL-10 gene family (Caudell et al., 2002; Huang et al., 2001). MDA-7/IL-24 contains seven exons and six introns. The cDNA of MDA-7/IL-24 is 1,718 base pairs and the protein encodes 206-amino acids (Huang et al., 2001). Being a secreted cytokine, MDA-7/IL-24 has a 49-amino acid N terminal hydrophobic signal peptide that allows for protein secretion (FIG. 16). Sauane and colleagues in the Fisher laboratory utilized the Prosite database to analyze the peptide sequence of MDA-7/IL-24 and identified three putative N-glycosylation sites at amino acid 85, 99 and 126 (Sauane et al., 2003b). In addition, an IL-10 signature motif was identified from amino acids 101 to 121, three protein kinase C consensus phosphorylation sites were identified at amino acids 88, 133 and 161 and three casein kinase II consensus phosphorylation sites were identified at amino acids 101, 111 and 161 using this database (Sauane et al., 2003b). The predicted tertiary structure of MDA-7/IL-24 is that of a compact globular molecule comprised of four strongly helical regions interspersed by loops of unpredicted structure (Sauane et al., 2003b). MDA-7/IL-24 can also form N-linked glycosylated dimers though intermolecular disulfide bonds and these dimers are functionally active (Mumm et al., 2006).

SPLICE VARIANTS/ISOFORMS OF MDA-7/IL-24. Allen and colleagues identified a splice variant of MDA-7/IL-24, mda-7s, which lacked exons 3 and 5 (Allen et al., 2004). They observed that MDA-7S could heterodimerize with full length MDA-7/IL-24 but noted that this interaction did not affect the apoptotic abilities of MDA-7/IL-24 in melanoma cells. Since the expression of mda-7s was reduced or absent in melanoma as compared to normal melanocytes, the authors also suggested an association between loss of mda-7s and metastatic melanoma (Allen et al., 2004). This same group also identified and published a short study on the presence of two splice variants that lacked exon 3 and exon 5, respectively, that were expressed in normal human melanocytes but not in metastatic melanoma (Allen et al., 2005). Filippov and colleagues identified splice isoforms of MDA-7/IL-24 while studying the effects of a ubiquitous splicing factor SRp55, that is upregulated by DNA damage in the absence of p53 and whose inactivation enhanced DNA damage resistance in a p53-dependent manner (Filippov et al., 2008). U2OS human osteosarcoma cells treated with siRNA to SRp55 were assessed using a splice-specific microarray analysis to identify the relevance of SRp55 on the splicing patterns of genes involved in apoptosis. At least 4 isoforms of MDA-7 were identified, out of which one isoform (that lacks exons 2 and 3) was sensitive to splicing by SRp55 and silencing SRp55 splicing activity caused an increase in this isoform. In a follow-up study, Whitaker and colleagues identified and characterized 5 alternatively spliced isoforms of MDA-7/IL-24 (Whitaker et al., 2011). Overall, they observed 6 differentially spliced transcripts of MDA-7/IL-24 in addition to the full-length transcript (FIG. 17). The splice variants identified were mda-7/IL-24δ3,5—lacking exons 3 and 5 (described and characterized previously by (Allen et al., 2004)); mda-7/IL-24δ5—lacking exon 5; mda-7/IL-24δ2,3—lacking exons 2 and 3; mda-7/IL-24δ2,5—lacking exons 2 and 5; mda-7/IL-24δ2,3,5—lacking exons 2, 3 and 5; and mda-7/IL-24δ2—lacking exon 2. All 7 exons were present in the full-length transcript. An important point to note is that the expression and distribution of mda-7/IL-24 isoforms might vary based on different cell-types. Full length MDA-7/IL-24 as well as spliced isoforms δ5, δ2,3,5, δ2,5 and δ2 were capable of reducing U2OS cell viability with no effect on the viability of non-cancerous immortalized NOK cells (Whitaker et al., 2011). Interestingly, in U2OS cells expressing mda-7/IL-24δ2,3,5 apoptosis was higher than cells expressing full length MDA-7/IL-24.

DELETIONS, MODIFICATIONS AND ENHANCING STABILITY OF MDA-7/IL-24. In an effort to identify the molecular basis of tumor-cell selectivity of MDA-7/IL-24, Gupta and colleagues in the Fisher laboratory constructed several amino terminal deletion mutants of MDA-7/IL-24 and labeled them M1 to M6 (Gupta et al., 2006b). The signal peptide was deleted in Ml; a-helical domain A was disrupted in M2; α-helical domain B was disrupted in M3; α-helical domains C, D, E and F were present in M4; α-helical domains D, E and F were present in M5; and α-helical domains E and F were present in M6. As would be expected, deletion of the signal peptide (M1) did not disrupt the tumor inhibitory effects of MDA-7/IL-24. Interestingly, however, all the other deletions, except M4, caused a loss of tumor inhibitory effects. M4 showed tumor suppressive effects in Hela and DU-145 cells but did not affect normal prostate epithelial P69 cells and was capable of inducing cancer cell-specific apoptosis (Gupta et al., 2006b).

MDA-7/IL-24 protein gets ubiquitinated and degraded via the 26S proteasome. In order to determine the exact site of ubiquitination, Tian and colleagues mutated each of the 10 lysine sites within the MDA-7/IL-24 protein and converted them to arginine (Tian et al., 2012). They identified lysine 123 as the critical internal lysine involved in MDA-7/IL-24 ubiquitination. Further conversion of lysine 123 to arginine was shown to enhance MDA-7/IL-24 protein stability as well as tumor suppressive abilities (Tian et al., 2012).

RECEPTORS OF MDA-7/IL-24. IL-10 cytokine family members signal through receptor dimers that consist of an R1 type receptor (with a long cytoplasmic domain) and an R2 type receptor (with a short cytoplasmic domain) The IL-10 cytokine family of receptors has three R1 and two R2 subunits. The R1 subunits are IL-10R1, IL-20R1 and IL-22R1 and the R2 subunits are IL-20R2 and IL-10R2. In order to identify the receptors of MDA-7/IL-24, Wang and colleagues utilized a biochemical approach using IL-24 affinity-tagged to the secreted human placental alkaline phosphatase (IL-24-AP) (Wang et al., 2002). They observed that MDA-7/IL-24 utilized two heterodimeric receptors, IL-22R1/IL-20R2 and IL20-R1/IL-20R2, to activate downstream signaling (Wang et al., 2002). Dumoutier and colleagues utilized ligand-dependent STAT (signal transducer and activator of transcription) activation as readout for receptor activation and independently identified these same receptors (Dumoutier et al, 2001). More recent studies by Dash and colleagues in the Fisher laboratory demonstrated that MDA-7/IL-24 can also signal and induce growth suppression and apoptosis in a cancer-selective manner using the IL-20R1/IL22-R1 heterodimeric receptors (Dash et al., 2014; Pradhan et al., 2017). However, the mechanism through which these two R1 receptor dimers promote signaling after interacting with MDA-7/IL-24 remains to be determined.

PHYSIOLOGICAL ROLE OF MDA-7/IL-24. Extensive studies were performed to understand the role of MDA-7/IL-24 in cancer, however, our understanding of the physiological role of MDA-7/IL-24 is fairly limited. In the following section, we discuss the cellular source of MDA-7/IL-24 and its functions in normal physiology.

NATURALLY OCCURRING CELLULAR SOURCE OF MDA-7/IL-24. MDA-7/IL-24 can be produced by immune cells (myeloid cells and lymphoid cells and monocytes) in response to treatment with lipopolysaccharides or specific cytokines (Buzas et al., 2011). Physiological levels of MDA-7/IL-24 are induced in Th2 lymphocytes by stimulation with phorbol myristate acetate (PMA) and Ionomycin and in T cells, especially CD4+naïve and memory cells activated by anti-CD3 monoclonal antibody (Sahoo et al., 2011; Schaefer et al., 2001). In monocytes, MDA-7/IL-24 is induced by antigenic stimulation with lipopolysaccharide, concanavalin A and cytokines (Caudell et al., 2002; Wolk et al., 2004). B cell receptor signaling also triggers MDA-7/IL-24 expression in B-lymphocytes (Maarof et al., 2010). Non-lymphoid cells can also produce physiological levels of MDA-7/IL-24 in response to cytokines secreted by immune cells (Persaud et al., 2016). Several in vitro and in vivo studies established that epithelial cells when stimulated with cytokines can secret MDA-7/IL-24 (Buzas et al., 2011; Persaud et al., 2016; Whitaker et al., 2012). Additionally, IL-1 can stimulate MDA-7/IL-24 expression in keratinocytes and human colon cells (Andoh et al., 2009). Basal expression of MDA-7/IL-24 at physiological levels is found in melanocytes and expression gradually decreases as the melanocytes begin to transform into metastatic melanoma (Ekmekcioglu et al., 2001; Ellerhorst et al., 2002; Jiang et al., 1995).

MDA-7/IL-24 FUNCTIONS UNDER PHYSIOLOGICAL CONDITIONS. MDA-7/IL-24 is produced by various immune cells and exerts a range of immune functions (Persaud et al., 2016). At lower physiological concentrations, MDA-7/IL-24 mainly functions as a cytokine. MDA-7/IL-24, when secreted, interacts with distinct sets of receptors including IL-20R1/IL-20R2, IL-22R1/IL-20R2 or IL-22R1/IL-20R1 receptor complexes (Dash et al., 2014; Dumoutier et al., 2001; Wang and Liang, 2005; Wang et al., 2002). Most immune cells lack the cognate pairs of receptors and chiefly express IL-20R2. One study by Caudell and colleagues assessed the secretion profile of peripheral blood mononuclear cells treated with MDA-7/IL-24 protein, which showed increased secretion of immune modulatory cytokines such as IL-6, IL-1(3, IFN-γ, TNF-α, IL-12 and GM-CSF (Caudell et al., 2002). The enhanced secretion of IFN-γ in turn upregulates IL-22R1 expression in keratinocytes, which facilitates formation of IL-22R1/IL-20R2 receptor pairs and induces innate immunity responses (Wolk et al., 2004). In addition to immune functions, MDA-7/IL-24 also induces several additional changes in normal skin cells. He and colleagues developed transgenic mice, which overexpress MDA-7/IL-24 specifically in skin (He and Liang, 2010). This genetically modified mouse is embryonic lethal and exhibits epidermal hyperplasia and abnormal keratinocyte differentiation. In contrast, treatment of human keratinocytes with MDA-7/IL-24 in a wound-healing model results in suppression of keratinocyte proliferation, suggesting a potential therapeutic role of this cytokine in proliferating skin lesions (Liang et al., 2011; Poindexter et al., 2010). MDA-7/IL-24 also impedes B cell maturation to plasma cells by regulating several transcription factors, which are important for plasma cell differentiation (Maarof et al., 2010). Additionally, MDA-7/IL-24 plays a diverse role in pro-inflammatory, infectious and autoimmune skin diseases, which is discussed in further detail below (Persaud et al., 2016).

Apart from these immune and dermatologic functions, several studies have also reported other biological functions of MDA-7/IL-24 in vascular diseases and inflammatory bowel disease (Persaud et al., 2016). MDA-7/IL-24 is also expressed in normal cultured fetal membranes, suggesting a potential role in normal pregnancy (Nace et al., 2010).

FUNCTIONAL ROLE OF MDA-7/IL-24 IN CANCER. The role of mda-7/IL-24 has been extensively studied in cancer. In this section, we describe briefly some of the important findings.

STEM CELLS AND DIFFERENTIATION. Tumors are comprised of heterogeneous cell populations with diverse biological properties. Cancer stem cells are immortal cells within tumors which display the property of self-renewal. They can divide and differentiate to give rise to a heterogeneous cell population, in which subsets of cells can form distant tumors (Talukdar et al., 2016). Stem cells detach from the primary tumor, migrate and generate tumors at distant sites. Cancer stem cells can relapse and metastasize making the need for specific therapies against them essential (Talukdar et al., 2016). They are also resistant to conventional therapies and divide more rapidly (Morrison et al., 2013).

mda-7/IL-24 inhibits the growth of breast cancer stem cells. Specifically, infection of Ad.mda-7 decreased proliferation of breast cancer initiating cells without harming normal stem cells (Bhutia et al., 2013). Over expression of mda-7/IL-24 induces apoptosis and ER stress in sorted stem cell populations of breast cancer cells, which is similar to what is observed in unsorted breast cancer cells (Bhutia et al., 2013). Over expression of mda-7/IL-24 also decreases the self-renewal capabilities of cancer stem cells. mda-7/IL-24 suppresses β-catenin/Wnt signaling (Chada et al., 2005; Sieger et al., 2004) and regulates the proliferation of stem cells. The Wnt/β catenin pathway is one of the key signaling pathways that promotes self-renewal of stem cells (Xu et al., 2016). Wnt proteins interact with Frizzled and LRP receptors to signal (β-catenin to activate Wnt target genes (MacDonald and He, 2012). It can also signal through ROR/RYK receptors as an alternative pathway (Green et al., 2014). In cancer, these are dynamically expressed and this causes an imbalance in the proliferation and differentiation of cancer stem cells. Alteration of the β-catenin signaling pathway increases the survival of stem cells. This suggests that mda-7/IL-24-mediated blockage in proliferation of stem cells is facilitated through the β-catenin pathway. In a subcutaneous human tumor xenograft nude mouse model, injection of Ad.mda-7 inhibited the growth of subcutaneous tumors. Tumor growth inhibition is associated with inhibition in cellular proliferation and angiogenesis (Bhutia et al., 2013).

Over expression of mda-7/IL-24 by an adenoviral system increased the expression of tumor suppressors including PTEN, E-cadherin, GSK-3β, and APC and down regulated proto-oncogenes involved in β-catenin and PI3K signaling (Gupta et al., 2006a). β-catenin translocates to the plasma membrane from the nucleus upon mda-7/IL-24 treatment, which reduces the transcriptional activity of TCF/LEF (Mhashilkar et al., 2003). This up-regulates the expression of E-cadherin-(β-catenin adhesion in a cancer-selective manner In lung and breast cancer, mda-7/IL-24 regulates cell-cell adhesion by modulating these signaling cascades (Mhashilkar et al., 2003). These effects are not common in normal cells and are specific for cancer cells.

Ad.mda-7 down regulates the tendency of breast cancer cells to form mammospheres and also inhibits the formation of distant tumors (Bhutia et al., 2013). A small proportion of stem cells are the progenitors of metastatic tumors, even after surgery, and they tend to be resistant to radiotherapy (Eyler and Rich, 2008). MDA-7/IL-24 regulates the PI3K/Akt pathway, decreases β-catenin phosphorylation and proteosomal degradation pathways (Bhutia et al., 2013; Mhashilkar et al., 2003). Stem cells also display over expression of Akt, Bcl2, and Bcl-xL (Wang and Scadden, 2015). mda-7/IL-24 can induce apoptosis by down regulating Akt, Bcl2, and Bcl-xL as described earlier (FIG. 18).

APOPTOSIS. Programmed cell death or apoptosis plays a pivotal therapeutic role in cancer drug sensitivity (Naik et al., 1996). One of the hallmarks of cancer is apoptosis (Hanahan and Weinberg, 2000). It involves a series of signaling events that are disrupted in cancer. Cancer cells bypass the apoptotic signaling pathway and evade this mechanism of cell death (Fernald and Kurokawa, 2013). Much of the research focusing on cancer therapeutics involves the ability of the therapy to induce apoptosis, specifically in cancer cells (Lebedeva et al., 2003a). Side effects of chemotherapy are due to non-selective toxicity toward normal cells. Understanding the mechanism by which cancer cells evade the general apoptotic pathways is critical to develop new therapies against cancer.

mda-7/IL-24 regulates ER (endoplasmic reticulum) stress and the mitochondrial apoptotic pathway (Fisher, 2005; Gopalkrishnan et al., 2004; Lebedeva et al., 2003c; Lebedeva et al., 2005a; Lebedeva et al., 2005b; Sauane et al., 2008; Sieger et al., 2004). Over expression of mda-7/IL-24 has been shown to induce apoptosis in different cancer cells without any harmful effect to normal cells (reviewed in Fisher, 2005). This cancer cell-specific death is both time- and dose-dependent. SB203580, an inhibitor of the p38MAPK pathway, inhibits Ad.mda-7-induced apoptosis. The p38MAPK or mitogen protein kinase pathway is altered due to over expression of mda-7/IL-24 (Sarkar et al., 2002b). This induces GADD genes (growth arrest and DNA damage) leading to cell cycle arrest and cell death (Sarkar et al., 2002b). AIF-mediated apoptosis by mda-7/IL-24 has recently been demonstrated to occur uniquely in neuroblastoma (Bhoopathi et al., 2016). A recent study from our group showed that mda-7/IL-24 regulates a subset of microRNAs (Pradhan et al., 2017). One microRNA, miR-221, was down regulated following treatment with mda-7/IL-24. miR-221 targets PUMA, a proapoptotic gene, blocking apoptosis (Pradhan et al., 2017). mda-7/IL-24 down regulates miR-221, which in turn up regulates PUMA inducing cell death (Pradhan et al., 2017). mda-7/IL-24 down regulates the expression of anti-apoptotic proteins Mcl-1, Bcl-xL, and Bcl2, while inducing pro-apoptotic proteins such as Bid, Bim, Bax, and Bak (Menezes et al., 2014). In so doing, mda-7/IL-24 increases the Bax/Bcl2 ratio (Pei et al., 2012). Previous studies also demonstrated a role of PERK in mda-7/IL-24-mediated cell death (Park et al., 2008).

Enhanced expression of mda-7/IL-24 induces the production of reactive oxygen species (ROS), which regulates multiple signaling cascades deregulating the mitochondrial integrity and cell death (Dent et al., 2010; Lebedeva et al., 2005b; Lebedeva et al., 2003c). The role of ROS in mda-7/IL-24 mediated cell death is well established. N-acetyl cysteine (Nace et al., 2010) inhibits cell death mediated by mda-7/IL-24 (Lebedeva et al., 2005a). Simultaneously, ROS inducers enhance cell death mediated by mda-7/IL-24 (Lebedeva et al., 2005b; Sauane et al., 2008). These results confirm the role of ROS and mitochondrial membrane potential as an important component in cell death promoted in cancer cells by the cytokine MDA-7/IL-24.

mda-7/IL-24 also up regulates SARI, a tumor suppressor, which is cancer-specific (Dash et al., 2014). Ectopic expression of mda-7/IL-24 induces SARI mRNA and protein in a broad panel of cancer cells (Dash et al., 2014). SARI expression is required for the anti-tumor effects of mda-7/IL-24. Recombinant MDA-7/IL-24 protein also induces SARI expression through binding to its cognate receptors, IL-20R1/IL-20R2/IL-22R1 (Dash et al., 2014).

The FasL signaling pathway is another pathway activated by Ad.mda-7, which results in cancer-cell selective apoptosis (Gopalan et al., 2005). Ad.mda-7 induces activation of the transcription factors c-Jun and ATF2 (activating transcription factor 2) inducing FasL-Fas (Gopalan et al., 2005). siRNA targeting Fas decreased mda-7/IL-24-induced cell death in ovarian cancer cells (Gopalan et al., 2005). This work reveals a role of mda-7/IL-24 in regulating the Fas-FasL signaling cascade to induce cancer cell death.

mda-7/IL-24 up regulates PKR (serine/threonine protein kinase) in non-small cell lung cancer, which is independent of p53 expression (Mhashilkar et al., 2003). The regulation of PKR by mda-7/IL-24 is post transcriptional (Gupta et al., 2006a). Exogenous recombinant mda-7/IL-24 also induces PKR and mda-7/IL-24 interacts with PKR in cancer cells (Pataer et al., 2005).

Apoptosis mediated by mda-7/IL-24 is independent of p53 mutations and functions (Gupta et al., 2006a; Su et al., 2003). It is established that mda-7/IL-24 induces apoptosis in diverse breast cancer cells, i.e., MCF7 (p53-wt), MDA-MB-231 (mutant p53), MDA-MB-453 (mutant p53), and T47D (mutant p53) (Chada et al., 2006). Based on different genetic backgrounds, these results indicate that cell death induction by mda-7/IL-24 is also independent of ER/PR/HER2 status in breast cancer cells. In these contexts, mda-7/IL-24-induced apoptosis is distinct from other identified tumor suppressors.

Secreted MDA-7/IL-24 protein functions as an anti-angiogenic molecule (Chada et al., 2004a; Nishikawa et al., 2004). It binds to its cognate receptor pairs and induces phosphorylation and nuclear translocation of STAT3 (Chada et al., 2004a). This receptor interaction induces BAX protein leading to cell death (Gupta et al., 2006a). This process is STAT3-independent as other interleukins (IL-10, IL-19, IL-20, and IL-22) also activate STAT3 without promoting cell death (Mosser and Zhang, 2008). mda-7/IL-24 binds IL-20/IL-22 receptor complexes resulting in activation of the JAK/STAT cascade. Studies have shown that mda-7/IL-24 induces apoptosis of cancer cells independent of the JAK/STAT pathway (Sauane et al., 2003a). Specifically, inhibitors of JAK/STAT pathway do not inhibit apoptosis mediated by mda-7/IL-24 (Sauane et al., 2003a). These results demonstrate that mda-7/IL-24 is independent of tyrosine kinase activation.

AUTOPHAGY. Autophagy is the process of degradation of organelles located in the cytoplasm. This process is complex owing to its differential context dependent role. “Is autophagy good or bad for life and cancer?” is a difficult question to answer (Bhutia et al., 2013). Sometimes it is protective, helping cancer cells to survive adverse conditions but it can also be toxic towards cancer cells (Bhutia et al., 2013; Liu and Debnath, 2016) (FIG. 19). Small molecules that can control autophagy may in certain contexts provide therapeutic benefit. Autophagy is a conserved phenomenon and it promotes tumor growth in advanced cases of cancer. mda-7/IL-24 induces autophagy, which is mediated by PERK (Park et al., 2008) and Beclin-1 (Bhutia et al., 2010). mda-7/IL-24 regulates a subset of microRNAs, including the oncogenic microRNA, miR-221 (Pradhan et al., 2017). Beclin-1 was identified as a new transcriptional target of miR-221 (Pradhan et al., 2017). mda-7/IL-24 down regulates miR-221, which in turn induces beclin-1, leading to autophagy (Pradhan et al., 2017). Cleavage of LC3, a marker of autophagy, is also observed. In renal and ovarian cancers, CD95 is an important regulatory molecule in the induction of autophagy mediated by mda-7/IL-24 (Park et al., 2009).

ANGIOGENESIS. Cancer and metastatic spread depends on an adequate supply of nutrients and oxygen to cells (Welch and Fisher, 2016). Additionally, removal of waste products also requires new blood and lymph vessels. The process of formation of new blood vessels is called angiogenesis, which represents another hallmark of cancer (Hanahan and Weinberg, 2011). Angiogenesis is regulated by a number of activator and inhibitor molecules. Although not as effective as anticipated when used as a single agent, angiogenesis inhibitors combined with other therapeutic agents are showing promise in the treatment of various cancers.

Over expression of mda-7/IL-24 in HUVEC cells or human umbilical vascular endothelial cells inhibits endothelial cell differentiation (Dash et al., 2010; Wang et al., 2016). Similarly, treatment of tumor xenografts with mda-7/IL-24 reduces expression of angiogenesis markers (Bhutia et al., 2012). VEGF (Vascular endothelial growth factor) and bFGF (basic fibroblast growth factor), which induce angiogenesis, are inhibited by MDA-7/IL-24 protein (Nishikawa et al., 2004). The PI3K/Akt pathway is another signaling cascade known to regulate angiogenesis (Karar and Maity, 2011) and mda-7/IL-24 down regulates phospho Akt and can therefore negatively modulate angiogenesis (Dash et al., 2010).

INVASION AND METASTASIS. mda-7/IL-24 has been shown to impede the migration of cancer cells (Ramesh et al., 2004). Also, over expression of mda-7/IL-24 results in a decrease in the in vitro invasion of an array of different cancer cell types (Ramesh et al., 2004). Lung cancer cells showed an inhibition in migration and invasion by modulating a number of signaling cascades (Panneerselvam et al., 2015). Focal adhesion kinase (FAK) and matrix metalloproteinases (MMPs) play a critical role in migration and invasion of cells (Hauck et al., 2002; Lin et al., 2000). mda-7/IL-24 down regulates FAK and MMP-2/MMP-9 protein, which indirectly inhibits migration and invasion of cancer cells (Menezes et al., 2014; Ramesh et al., 2004). mda-7/IL-24 has been shown to promote potent anti-invasive activity in lung cancer cells, cervical cancer cells, and liver cancer cells (Emdad et al., 2009; Lebedeva et al., 2007). mda-7/IL-24 regulates a number of molecules related to metastasis, i.e., cyclin-B1, TGF-β, Survinin, Twist, ICAM-1, and CD44 (Huo et al., 2013). Also, E-cadherin, NF-KB, and PERK are regulated by mda-7/IL-24 (Panneerselvam et al., 2013). mda-7/IL-24-mediated inhibition in invasion and metastasis is both receptor-dependent and receptor-independent (Menezes et al., 2014).

SYNERGISTIC EFFECTS. Cancer is a complex process that is mediated by multiple genetic and epigenetic changes that impact directly and indirectly on a number of pivotal signaling pathways involved in cell growth, survival, resistance to apoptosis, and additional physiologically relevant processes (Hanahan and Weinberg, 2000, 2011). Considering this complexity, it is not surprising that a single targeting molecule fails to provide complete therapy resulting in a cure in most cancers. Conversely, a combinatorial approach using multiple target-selective agents directed toward specific signaling abnormalities in defined cancers have shown promise in cancer therapy.

Based on Phase I/II clinical studies mda-7/IL-24 has been shown to have a therapeutic role in cancer (Cunningham et al., 2005; Fisher et al., 2003; Fisher et al., 2007; Tong et al., 2005). Also, pre-clinical studies have confirmed synergistic therapeutic responses when mda-7/IL-24 is combined with existing therapies, including radiation, chemotherapy, antibody-based therapies, small molecule and immunotherapies) (Table 1). The mechanisms underlying this synergy include the regulation of similar pathways as well as different pathways by mda-7/IL-24. This is tabulated in Table 1.

TABLE 1
Combinatorial enhancement of therapy by combining
mda-7/IL-24 with other therapeutic modalities.

| Therapeutic Agent | Cancer types | References |
|---|---|---|
| Trastuzumab | Breast cancer | (Li et al., 2015; McKenzie |
| | | et al., 2004) |
| Bevacizumab | Lung cancer | (Inoue et al., 2007) |
| Erlotinib | Melanoma | (Deng, Kwon et al. 2011) |
| Gefitinib | NSCLC | (Emdad et al., 2007a) |
| Temozolamide | Glioblastoma | (Hamed et al., 2010b) |
| Tarceva | NSCUC | (Gupta et al., 2008) |
| Arsenic Trioxide | Renal carcinoma | (Yacoub et al., 2003) |
| Cisplatin | Liver, Colorectal | (Wu et al., 2009) |
| Sabutoclax (Mcl-1 | Prostate | (Dash et al., 2011) |
| inhibitor) | | |
| Sabutoclax (Mcl-1 | Colorectal | (Azab et al., 2012) |
| inhibitor) | | |
| BI-97D6 (Mcl-1 | Prostate | (Sarkar et al., 2015b) |
| inhibitor) | | |
| Grp170 | Prostate | (Gao et al., 2008a) |
| Radiation | Prostate | (Su et al., 2006) |
| BI-69A11 | Colon | (Pal et al., 2014) |
| 5-FU | Esophageal | (Ma et al., 2014) |
| HSP90 inhibitors | Pancreatic | (Zhang et al., 2013) |
| HDAC inhibitors | Renal carcinoma | (Hamed et al., 2013a) |
| HDAC inhibitors | Glioblastoma | (Hamed et al., 2013b) |
| 5-FU, Doxorubicin | Colon | (Xu et al., 2013) |
| Sorafenib | Renal carcinoma | (Eulitt et al., 2010) |
| Doxorubicin | Hepatocellular | (Wang et al., 2010) |
| | carcinoma | |
| Dichloroacetate | Hepatocellular | (Xiao et al., 2010) |
| | carcinoma | |
| osu-03012 | Glioblastoma | (Hamed et al., 2010a) |
| Perillyl alcohol | Pancreatic cancer | (Lebedeva et al., 2008) |
| CDDP, Epirubicin, VCR | B cell lymphoma | (Ma et al., 2016a) |
| Doxorubicin | Colorectal | (Emdad et al., 2007b) |
| Vit E Succinate | Ovarian cancer | (Shanker et al., 2007) |
| Geldanamycin | Lung cancer | (Pataer et al., 2007) |
| Radiation | Ovarian | (Emdad et al., 2006) |
| Celecoxib | Breast cancer | (Suh et al., 2005) |
| Sulindac | Lung cancer | (Oida et al., 2005) |

BYSTANDER ACTIVITY. Evidence of bystander activity of mda-7/IL-24 (Su et al., 2005) was shown in vivo in animal studies, where tumor cells were injected in both flanks of nude mice (Pradhan et al., 2017; Sarkar et al., 2007; Sarkar et al., 2008; Sarker et al., 2005; Su et al., 2005). A tumor on one flank was treated while the tumor on the other flank was left untreated. Tumor measurements showed a decrease in tumor size in the treated as well as the untreated tumor. The inhibitory action on distant tumors can be explained by the anti-tumor “bystander” activity of the secreted mda-7/IL-24 cytokine and its ability to induce apoptosis and promote production of MDA-7/IL-24 through dimeric receptor pairs in the untreated tumor (Menezes et al., 2014; Sauane et al., 2008). Additionally, in a syngeneic model this distant anti-tumor effect can also be explained by the activation of immune pathways, i.e., cytotoxic T cells and NK cells by administration of mda-7/IL-24 (Gao et al., 2008; Menezes et al., 2015; Miyahara et al., 2006). Over expression of mda-7/IL-24 gene results in production of MDA-7/IL-24 protein which is secreted as a glycosylated protein (Dash et al., 2010; Fuson et al., 2009; Sauane et al., 2006). Infection of Ad.mda-7, which is dependent on CAR (Coxsackie and adenovirus) viral receptors on cells, or treatment with GST-MDA-7 are not dependent on the IL-20R1/IL-20R2/IL-22R1 receptors (Dent et al., 2010; Sauane et al., 2004). In contrast, to provoke a signaling and biological effect, secreted MDA-7/IL-24 requires a complete set of dimeric cell surface receptors (Dash et al., 2014; Dumoutier et al., 2001; Wang et al., 2002). Secreted MDA-7/IL-24 binds to the dimeric receptor pair and induces cancer cell death (Dash et al., 2014; Menezes et al., 2014). By the use of IL-20R1/IL-20R2 antibodies, it has been demonstrated that mda-7/IL-24-mediated cell death is receptor-dependent (Chada et al., 2004a). Zheng and colleagues described the role of IL-20R1/IL-20R2 receptor pair in mda-7/IL-24-mediated cell death and its independence of STATS phosphorylation (Zheng et al., 2007a). Biological activity of mda-7/IL-24 was also shown to be independent of JAK/STAT signaling using inhibitors and various receptor mutant cells (Sauane et al., 2003a).

Normal cells also promote “bystander” activity after exposure to mda-7/IL-24, which results in production and secretion of MDA-7/IL-24 without inducing toxicity or cell death. Infection of normal primary or immortal human cells, such PHFA, FM-516 or P69, results in secretion of MDA-7/IL-24. Addition of supernatant from normal cells infected with Ad.mda-7 to cancer cells results in suppression of their growth and induction of apoptosis. Since Ad.mda-7 will result in MDA-7/IL-24 protein production in normal and cancer cells, this can result in a robust “bystander” effect that is observed both in pre-clinical animal models and in a Phase I/II clinical trial in patients with advanced cancers (Dash et al., 2010).

Activation of the immune system provides another important mechanism underlying the “bystander” activity of mda-7/IL-24. MDA-7/IL-24 induces IL-6, TNF-α, IFN-γ, IL-1β, and IL-12, which are potent immunoregulatory molecules (Caudell et al., 2002; Deng et al., 2011; Menezes et al., 2014). Also, these immunoregulatory molecules can regulate APCs to present tumor antigens to trigger immune response (Gupta et al., 2006a) (I do not think this is the correct reference- please check Gupta references--). In addition to immune mediated effects, the “bystander” antitumor activity of MDA-7/IL-24 is also elicited through its direct proapoptotic and anti-angiogenic activity (Dash et al., 2010).

Role of MDA-7/IL-24 in other diseases. MDA-7/IL-24 has been extensively studied in cancer. In addition to its function as a tumor suppressor and apoptosis-toxic autophagy inducing cytokine in cancer, MDA-7/IL-24 has also been reported to play a significant role in inflammation, cardiovascular disease, autoimmune diseases and viral replication.

Inflammation. The skin is the largest organ in the body and plays an essential role in promoting immunity and defense against pathogenic microorganisms. However, dysregulated immune reactions can cause chronic inflammatory skin diseases. Extensive crosstalk between the different cellular and microbial components of the skin regulates local immune responses to ensure efficient host defense, to maintain and restore homeostasis, and to prevent chronic disease. In this section, we briefly discuss recent findings that highlight a role of MDA-7/IL-24 in inflammation. IL-19 and MDA-7/IL-24 belong to the IL-20 subfamily and are known to be involved in host defense against bacteria and fungi, tissue remodeling and wound healing (Fonseca-Camarillo et al., 2014). These groups of cytokines are involved in protecting the epithelial tissue from damage that is a consequence of bacterial and viral infections. MDA-7/IL-24 may be a member of a complex cascade of cytokines involved in inflammation as MDA-7/IL-24 can induce expression of many cytokines, including TNF-α, IL-6 and interferon-γ (IFN-γ) (Wang and Liang, 2005). MDA-7/IL-24 and its receptor expression pattern supports a major physiological function related to epidermal functions, such as wound healing, and abnormalities may be part of the cause of pathological skin conditions such as psoriasis.

Inflammatory bowel disease. Chronic inflammation of all parts of the digestive tract may bring about inflammatory bowel disease (IBD). This includes primarily ulcerative colitis and Crohn's disease. The symptoms for both of these conditions include severe diarrhea, pain, fatigue and weight loss. Genomic abnormalities and environmental factors can trigger IBD. Andoh and colleagues assessed the expression of MDA-7/IL-24 in inflamed mucosa of IBD patients and determined the molecular mechanism that resulted in MDA-7/IL-24 expression in colonic subepithelial myofibroblasts (Andoh et al., 2009). They demonstrated that MDA-7/IL-24 expression is enhanced in the inflamed mucosa of active IBD patients. Their data suggest that IL-24 targets epithelial cells and plays anti-inflammatory and protective roles in the intestinal mucosa. This elevated expression of MDA-7/IL-24 leads to increased Jak/Stat pathway signals leading to increased expression of different MUC genes in the mucosa. MUC genes are the primary component of the mucin barrier that divides the intestinal microbiota and the intestinal epithelium. MUC genes also play an important role in the pathogenesis of IBD. This study showed that IL-24 expression is elevated in inflamed mucosa of IBD patients compared to control patients. Work done by other researchers show that the IL-10 subfamily of cytokines is involved in immune regulation and inflammatory responses. To obtain an enhanced understanding of this group of cytokines for potential therapeutic applications, more focus is required on mechanism; some of them may in the future reduce adverse side effects and/or increase the efficacy typically observed in IL-10 therapy for IBD. In active IBD, MDA-7/IL-24 is synthesized by peripheral B cells, CD4+ T cells, CD8+ T cells and monocytes. Overall, MDA-7/IL-24 can promote a suppressive inflammatory effect on colonic epithelial cells and mucosal inflammation in IBD.

Studies by Fonseca-Camarillo and colleagues explored the role of MDA-7/IL-24 in Mexican matzo patients with IBD (Fonseca-Camarillo et al., 2014). The authors studied a total of 113 patients that included 77 patients with ulcerative colitis (UC) and 36 patients with Crohn's disease (CD). This study also included 33 patients as control. They compared the gene expression profiles of IL-19 and MDA-7/IL-24 in these patients. The study found that IL-19 and MDA-7/IL-24 levels were elevated significantly with active IBD disease compared with inactive IBD at both a transcriptional and translational level. Additionally, they showed that when compared with active ulcerative colitis and non-inflammatory tissue an increasd in IL-19 and MDA-7/IL-24 producing cells were observed in active Crohn's disease. This study indicates that in patients with active IBD, circulating B cells and monocytes produce IL-19 and peripheral B cells, CD4+ T cells, CD8+ T cells and monocytes produce MDA-7/IL-24.

Psoriasis. Psoriasis is a common chronic inflammatory skin disease resulting from a complex interplay among the immune system, keratinocytes, susceptibility genes, and environmental factors with a prevalence of 2% in the Caucasian population. Kumari and colleagues observed the presence of MDA-7/IL-24 as well as IL-19 and IL-20 in psoriatic skin lesions (Kumari et al., 2013). Results from these studies showed that MDA-7/IL-24 was elevated in psoriatic skin compared to normal skin. It is also reported that MDA-7/IL-24 can induce different psoriasis-associated factors, which can promote inflammation and epidermal hyperplasia (Kumari et al., 2013).

The interleukin 10 family of cytokines including MDA-7/IL-24 has been implicated in the pathogenesis of psoriasis (Kunz et al., 2006; Leng et al., 2011; Romer et al., 2003; Weiss et al., 2004; Wolk et al., 2009). These reports also showed an increased expression of MDA-7/IL-24 in psoriatic skin compared to normal skin. MDA-7/IL-24 was mainly produced by keratinocytes, myeloid cells, and T cells (Conti et al., 2003; Kunz et al., 2006; Zheng et al., 2007b). High expression of MDA-7/IL-24 receptors are also found in keratinocytes and they signal by activating STAT3 (Dumoutier et al., 2001; Kunz et al., 2006; Parrish-Novak et al., 2002). STAT3 over expression is also observed in psoriatic skin conditions and the expression of constitutively active STAT3 in epidermal keratinocytes also caused psoriasis-like skin inflammation in mice (Sano et al., 2005), which suggests an important role for epidermal STAT3 signaling in psoriasis (Kumari et al., 2013).

Kumari and colleagues report that epidermis-specific NF-κB inhibition increased MDA-7/IL-24 and STAT3 expression in keratinocytes in a TNFR1-dependent manner in psoriasis-like skin inflammation. In the psoriasis epidermis, MDA-7/IL-24 expression was elevated and inhibition of NF-κB increased MDA-7/IL-24 expression in TNF-stimulated human primary keratinocytes. This suggests the importance of this molecular pathway in human psoriasis. They also showed a new keratinocyte-intrinsic mechanism that linked TNFR1, NF-κB, ERK, MDA-7/IL-24, IL-22R1, and STAT3 signaling to disease initiation in psoriasis pathogenesis. The authors also show that skin inflammation requires both TNFR 1 signaling in IKK2-deficient epidermal keratinocytes and also identified skin epithelial cells as the major cellular target of this model. This manuscript also demonstrates that in keratinocytes, TNFR1-induced, ROS-, and ERK-dependent expression of MDA-7/IL-24 is a key early event in skin infammation. In the inflammatory process epidermis specific inhibition of NF-κB activates Stat3 and increases MDA-7/IL-24 expression in primary keratinocytes (Persaud et al., 2016). Taken together, the studies on MDA-7/IL-24 in psoriasis indicate a significant role in the expression of pro-inflammatory mediators thereby resulting in psoriatic skin lesions. The studies also provide evidence suggesting that MDA-7/IL-24 may play a key role in psoriasis initiation.

Cardiovascular Disease. Vascular calcification is a symptom of cardiovascular disease. Wang and colleagues showed that low concentration of H202 treatment induced abnormal proliferation of vascular endothelial cells and MDA-7/IL-24 inhibited this proliferation (Wang et al., 2016). They also showed that MDA-7/IL-24 could inhibit apoptosis by inhibiting ROS production in vascular endothelial cells. MDA-7/IL-24 is also involved in the down regulation of several genes that regulate cardiovascular disease. The authors concluded that MDA-7/IL-24 can provide a basic therapeutic strategy for treating vascular disease and cancer by inhibiting ROS production in vascular cells. Lower levels of MDA-7/IL-24 were observed in hypertensive rats compared to controls, and anti-hypertensive therapy increased MDA-7/IL-24 levels. Hypertension is also a hallmark of cardiovascular disease. MDA-7/IL-24 was identified as one of the 16 differentially regulated genes in spontaneously hypertensive rats. MDA-7/IL-24 also regulates the expression of inflammation- and hypertension-related genes in a H202-treated mouse vascular smooth muscle cell line, MOVAS. This study also showed that MDA-7/IL-24 attenuates H2O2-induced activation of PI3K/Akt and Erk. Studies by Ki-Mo Lee and colleagues also suggests that MDA-7/IL-24 can inhibit ROS production by regulating mitochondrial ROS release mediated by PI3K/Akt and Erk pathway in H2O2-treated vascular smooth muscle cells, VSMC's (Lee et al., 2012). This inhibition of ROS in VSMC leads to reduced cell growth and migration. Another study by Chen and colleagues also indicated that adenovirus-mediated expression of MDA-7/IL-24 could inhibit pulmonary arterial smooth muscle cell line (PAC1-SMC) migration and proliferation, leading to reduced intimal hyperplasia (Chen et al., 2003). This study also emphasizes the role of MDA-7/IL-24 in cancer-specific cell death as the authors validated the inhibition of proliferation and induction of apoptosis in PAC1-SMCs (these cells have tumorigenic potential) compared to normal human coronary artery SMC and rat aortic SMC. Based on this data MDA-7/IL-24 could be used as a therapeutic option for vascular proliferative disorders. Taken together, these studies suggest that MDA-7/IL-24 may be a novel therapeutic target for cardiovascular disease and/or hypertension.

Another study showed that MDA-7/IL-24 inhibits β-GP-induced vascular smooth muscle cell calcification. Activation of the Wnt/β-catenin pathway by β-GP is inhibited by MDA-7/IL-24, which indicates that the inhibitory effect of MDA-7/IL-24 on VSMC calcification correlates with the inactivation of the Wnt/β-catenin pathway. Although the authors did not explore the role of Jak/Stat pathway mediated β-GP-induced VSMC calcification by MDA-7/IL-24 in this study, they showed an effect of MDA-7/IL-24 on inhibition of the Wnt/β-catenin pathway using a neutralizing antibody to MDA-7/IL-24. This inhibition by MDA-7/IL-24 correlates with suppression of apoptosis, and the expression of osteoblast markers and calcification by down regulation of BMP-2 and the Wnt/β-catenin pathway. They also showed that β-GP increased the expression of calcification and osteoblastic markers in VSMCs (Persaud et al., 2016). This effect is specifically inhibited by MDA-7/IL-24 suggesting that MDA-7/IL-24 suppresses downstream molecules by inhibiting BMP-2 expression. The inhibitory effect MDA-7/IL-24 on VSMC calcification is mediated at least in part through anti-apoptotic activity. The effect of MDA-7/IL-24 on VSMC calcification is similar to statins, which are hydroxy-3-methylglutaryl coenzyme A reductase inhibitors. These results explain the role of MDA-7/IL-24 in pathophysiology of vascular calcification.

Vargas-Alarcon and colleagues showed in a case-control association study that individuals with premature coronary artery disease (CAD), subclinical atherosclerosis (SA), and healthy controls who had several metabolic and cardiovascular risk factors was associated with MDA-7/IL-24 polymorphisms (Vargas-Alarcon et al., 2014). The authors used an informatics approach and showed that the rs1150253 and rs1150258 polymorphisms in MDA-7/IL-24 had a functional effect generating DNA binding sites for transcription factors. In Mexican populations, these polymorphisms can be used as risk factors for cardiovascular disease, hypertension, diabetes and increased levels of lipids. The authors concluded that the association of MDA-7/IL-24 polymorphisms with metabolic parameters and cardiovascular risk factors was due to characteristic genetic background with important differences in Mexican populations compared to other populations.

Based on the available literature, MDA-7/IL-24 appears to play a distinct role in cardiovascular disease. MDA-7/IL-24 can promote the growth of vascular smooth muscle cells by suppressing calcification and osteoblast marker expression, which is associated with atherosclerosis pathogenesis. MDA-7/IL-24 also may provide benefit in the treatment of vascular disorders since it selectively inhibits rat pulmonary arterial smooth muscle cell growth and migration. Polymorphisms in the MDA-7/IL-24 gene also correlate with cardiovascular and metabolic risk factors, further supporting a relationship between MDA-7/IL-24 and cardiovascular diseases.

Rheumatoid arthritis (RA). Rheumatoid arthritis (RA) is an inflammatory auto-immune disease that can lead to progressive joint damage and disability. Cytokines including IL-1, IL-6, IL-8, IL-10, monocyte chemo-attractant protein 1 (CCL2/MCP-1), and tumor necrosis factor (TNFα) play an important role in RA. A study in RA and spondyloarthropathy (SpA) patients with osteoarthritis (OA) patients as controls, analyzed the role of IL-20 and MDA-7/IL-24 by measuring levels of expression, cellular sources, and targets and effects on cytokine production. This study indicated increased levels of IL-20 and MDA-7/IL-24 in RA and SpA patients as compared with inflammatory disease controls and normal controls. They also found that MDA-7/IL-24 levels were almost 10-times greater in these samples as compared to IL-20 levels in synovial fluid, demonstrating the dominant role of MDA-7/IL-24 locally in the joints, because these two cytokines share the same receptors. This study also showed that IL-20R1 and IL-22R are expressed in granulocytes from the RA and SpA patients' synovial fluid. This indicates that these two cytokines could be involved in neutrophil chemotaxis in arthritis. This study also showed that IL-20 and MDA-7/IL-24 are not involved directly in TNF-alpha and IL-6 production in arthritis, whereas increased expression of CCL2/MCP-1 in SFMC cultures was evident indicating a positive correlation in RA and SpA patients. Taken together, this study demonstrates the association of IL20 and MDA-7/IL-24 to the synovium of RA and SpA. It also implicates the importance of IL-20 and MDA-7/IL-24 in endothelial cell function and recruitment of granulocytes and mononuclear cells to the synovial joint (Kragstrup et al., 2008).

Kragstrup and colleagues observed an increased plasma concentration of IL-20 and MDA-7/IL-24 in early RA patients as compared to normal healthy controls, and with conventional or anti-inflamatory treatment these levels decreased (Kragstrup et al., 2008). Radiographic progression of the disease and the association of IL-20 and MDA-7/IL-24 suggest an involvement of these cytokines in bone destruction. These two cytokines link RA-associated autoantibodies and radiographic progression of IL-22R1. By showing the relationship between IL-20 and MDA-7/IL-24 and RA-associated immune complexes (ICs) and osteoclasts (OCs) stimulation via IL-22R1, the investigators demonstrate a correlation between the IL-20R axis and they also provide evidence for a relationship between the IL-20R axis and progression of structural damage. This study showed that targeting the IL-20R axis could be a viable treatment option for bone destruction in rheumatic disease. It also suggests that the dual inhibition of IL-20 and MDA-7/IL-24 or inhibition of IL-22R1 could be helpful in seropositive RA. These changes in this IL-20R axis provide a promising treatment modality for RA. These studies show a clear association of MDA-7/IL-24 and RA, though additional research is vital to fully understand the potential role of MDA-7/IL-24 in RA.

Tuberculosis. Tuberculosis (TB) is an infectious disease caused by Mycobacterium tuberculosis in humans. Although the lungs are the primary organs altered by TB infection, other parts of the body can also be affected. Wu and colleagues reported that active TB patients had decreased expression of MDA-7/IL-24 compared to individuals with latent TB infection. This observation led them to investigate the role of MDA-7/IL-24 in pulmonary TB patients. Since IFN-γ plays an important role in TB infection, and the levels of IFN-γ0 were similar to MDA-7/1L-24 levels in these patients, they investigated the role of MDA-7/IL-24 on IFN-γ expression. PBMCs isolated from these individuals were stimulated with Mycobacterium tuberculosis early secreted Ag of 6 kDa (EAST-6) to determine the levels of gene expression . Exogenous MDA-7/IL-24 in the presence of EAST-6 stimulation in PBMCs increased IFN-γ levels and neutralizing MDA-7/IL-24 decreased IFN-γ. This upregulation of IFN-γ with exogenous MDA-7/IL-24 was due to increased levels of IL-12α, IL-12β, IL-23α and IL-27. Taken together, these results show that MDA-7/IL-24 regulates IFN-γ in TB patients and targeting MDA-7/IL-24 might be a treatment option for these patients. Another study by Kumar and colleagues indicated significantly lower levels of MDA-7/IL-24 in TB patients (Kumar et al., 2015; Ma et al., 2011). Additional research is required in this area to decipher molecular mechanism of MDA-7/IL-24 in the pathophysiology of TB in patients.

Influenza virus replication. Influenza infection also known as flu, is associated with mild to severe symptoms including fever, headaches, runny nose and fatigue. Weiss and colleagues studied the role of MDA-7/IL-24 in Influenza A virus replication, as MDA-7/IL-24 is known to influence TLR3-mediated apoptosis and influenza virus can stimulate the TLR3 receptor (Weiss et al., 2015). In this study, the investigators demonstrated that the expression of MDA-7/IL-24 could decrease influenza A virus subtypes replication by inducing apoptosis. The reduction of viral replication by MDA-7/IL-24 could be independent of type I interferon. MDA-7/IL-24 could inhibit Mcl1 and induce caspase 3 cleavage due to initiation of TLR3-mediated apoptosis. This was further demonstrated by TLR3 knockdown or by treating cells with a Pan-Caspase inhibitor. Inhibition of anti-apoptotic proteins Bcl2, Bax, and Bcl-xL was also observed following MDA-7/IL-24 expression. They established that Mcl1 is the key factor in MDA-7/IL-24-mediated inhibition of influenza A virus replication. They also showed that MDA-7/IL-24 expressed by influenza A virus vector does not have any toxicity in mice. Another study by Seong and colleagues also showed that MDA-7/IL-24 expression decreased influenza viral replication (Seong et al., 2016). In this study, the authors also observed that Influenza virus infection regulated MDA-7/IL-24 expression. MDA-7/IL-24 decreased the transcript level of the viral nucleoprotein (NP) gene following influenza virus infection as compared to viral infection alone, confirming an inhibitory role of MDA-7/IL-24 in viral replication. Furthermore, an MDA-7/IL-24 expressing recombinant adenovirus did not induce toxicity as compared to a wild type adenovirus, suggesting that MDA-7/IL-24 can specifically target virus infected cells. Taken together, these studies suggest that MDA-7/IL-24 exerts potent inhibitory activity of influenza viral replication and can be used as a promising novel approach to suppress viral infections (Seong et al., 2016; Weiss et al., 2015).

IMMUNOLOGICAL EFFECTS OF MDA-7/IL-24. The role of MDA-7/IL-24 in normal physiology and disease pathology is quite diverse and depends principally on the source of production/secretion, and the target tissue. As a cytokine, MDA-7/IL-24 exerts immune-modulatory functions in diverse autoimmune, infectious and immuno-pathological diseases including Rheumatoid arthritis, Psoriasis, Inflammatory bowel diseases and others, as discussed in detail above (also reviewed in Persaud, De Jesus et al. 2016). MDA-7/IL-24 also plays a prominent role in host defense by inducing innate immune response in epithelial tissue during infection and inflammation by induction of chemokines and recruitment/activation of leukocytes (Jin et al., 2014; Tamai et al., 2012).

Apart from these immune-modulatory roles in diverse biological diseases, MDA-7/IL-24 also exerts a profound immune stimulatory effect in the context of cancer. Forced expression of MDA-7/IL-24 induces IFN-γ and IL-6 secretion from melanoma cells and displays potent anti-tumor functions (Caudell et al., 2002; Chada et al., 2004b). Transduction of MDA-7/IL-24 via an adenoviral vector resulted in a significant increase in the CD3+ and CD8+ population, thereby facilitating immune activation and antitumor immunity. In one recent study, Ma et al. evaluated the efficacy of MDA-7/IL-24 in inhibiting colon cancer progression in murine models with an intact immune system and explored the immune-modulatory role of MDA-7/IL-24 in colon cancer progression (Ma et al., 2016b). The investigators found that MDA-7/IL-24 promoted CD4+ CD8+ T cells to secrete IFN-γ0 and facilitated the cytotoxicity of CD8+ T cells. In another recent study, Menezes and colleagues in the Fisher laboratory assessed the relevance of immune response in MDA-7/IL-24-mediated tumor suppression in a transgenic murine mouse model of breast cancer with an intact immune system (Menezes et al., 2015). The investigators found that intratumoral injection of Ad.5-CTV (replication competent cancer-selective adenovirus expressing MDA-7/IL-24; a Cancer Terminator Virus) resulted in a marked increased IFN-γ expression and intra-tumoral CD8+ T cell infiltration. Interestingly, a significant increase in infiltrating CD8+9 T cells, along with increased IFN-γ and granzyme B expression was also observed in non-treated tumors derived from MMTV-PyMT transgenic mice that received Ad5-CTV suggesting that MDA-7/IL-24 is capable of inducing a systemic immune response in an intact immune microenvironment (Menezes et al., 2015). Another study by the Wang and Fisher laboratories evaluated the therapeutic efficacy of Ad.mda-7 in combination with an endoplasmic reticulum resident chaperone grp170 (Ad.sgrp170) in a prostate cancer model (Gao et al., 2009; Gao et al., 2008). The investigators demonstrated that the combination treatment of MDA-7/IL-24 and grp170 was more effective in inhibiting TRAMP-C2 prostate tumor growth as compared to a single agent. The combination treatment resulted in increased IFN-γ production and cytolytic activity suggesting an antigen and tumor-specific T-cell response. Interestingly, the combination treatment was able to reduce distant tumor burden suggesting induction of profound “bystander” systemic antitumor immunity (Gao et al., 2009; Gao et al., 2008). Additionally, a vaccine effect was evident with subsequent tumor challenge experiments associated with a significant increase in the CD3+ and CD8+ cell populations. All of these studies in diverse cancer models strongly support an anticancer immune modulatory role of MDA-7/IL-24.

Evidence of immune activation was also evident in a Phase I/II clinical trial of Ad.mda-7 (INGN-241) in patients with advanced cancers (Cunningham et al., 2005; Dash et al., 2010; Fisher et al., 2007; Sarkar et al., 2007; Tong et al., 2005). A majority of the patients receiving intermediate- or high-dose injections of Ad.mda-7 (INGN-241) showed a marked increase in CD3+ and CD8+ T cells at day 15 following injection as well as transient increases in circulating cytokines, such as IL-6, IL-10 and TNF-α (Cunningham et al., 2005; Tong et al., 2005). A few patients showed elevated levels of GM-CSF and IL-2 as well. These immune and cytokine profiles following injection of Ad.mda-7 (INGN-241) in patients mimic a TH-1 type immune response and strongly support an immune stimulatory function of MDA-7/IL-24 in eliciting an antitumor response.

CONCLUSIONS AND FUTURE PERSPECTIVES. As described in this review, MDA-7/IL-24 plays significant roles in a number of different human diseases. When initially identified, MDA-7/IL-24 was primarily recognized for its role as a tumor suppressor in cancer. However, as more information regarding the role of MDA-7/IL-24 became available our understanding of its relevance in other diseases has also increased. A detailed understanding of the molecular mechanisms defining the function of MDA-7/IL-24 have helped develop several preclinical therapeutic options as well as therapeutic targets against cancer. As mentioned previously, MDA-7/IL-24 has already been tested in clinical trials for cancer and a Phase I clinical trial with MDA-7/IL-24 (INGN 241) showed promising results (Cunningham et al., 2005; Tong et al., 2005). Currently, research is focused on developing novel approaches to enhance MDA-7/IL-24 potency and tumor specific delivery. The search for new molecules and compounds that can enhance or stabilize MDA-7/IL-24 protein are also ongoing. Finally, combination therapies that would enhance MDA-7/IL-24-mediated tumor cell killing and prevent tumor growth and metastasis are also being identified and tested preclinically (Menezes et al., 2014). As new MDA-7/IL-24 therapeutic options are developed in one disease indication, they will also be valuable against other human diseases with MDA-7/IL-24 involvement. Further information gained regarding the role of MDA-7/IL-24 in diseases where MDA-7/IL-24 is over expressed will allow researchers and clinicans to develop newer approaches to manage these conditions. Given the currently known functions of MDA-7/IL-24, it is likely that MDA-7/IL-24 will also be implicated in other disease indications. Such information will be critical for understanding the multifaceted role of MDA-7/IL-24 in human physiology.

### REFERENCES

Example 1 references:

1. Jiang H, Lin J J, Su Z Z, Goldstein N I, Fisher P B. Subtraction hybridization identifies a novel melanoma differentiation associated gene, mda-7, modulated during human melanoma differentiation, growth and progression. Oncogene. 1995;11(12):2477-86.

2. Jiang H, Su Z Z, Lin J J, Goldstein N I, Young C S, Fisher P B. The melanoma differentiation associated gene mda-7 suppresses cancer cell growth. Proceedings of the National Academy of Sciences of the United States of America 1996;93(17):9160-5.

3. Emdad L, Lebedeva I V, Su Z Z, Gupta P, Sauane M, Dash R, et al. Historical perspective and recent insights into our understanding of the molecular and biochemical basis of the antitumor properties of mda-7/IL-24. Cancer biology & therapy 2009;8(5):391-400.

4. Lebedeva I V, Sauane M, Gopalkrishnan R V, Sarkar D, Su Z Z, Gupta P, et al. mda-7/IL-24: exploiting cancer's Achilles' heel. Molecular therapy: the journal of the American Society of Gene Therapy 2005;11(1):4-18.

5. Dash R, Bhutia S K, Azab B, Su Z Z, Quinn B A, Kegelmen T P, et al. mda-7/IL-24: a unique member of the IL-10 gene family promoting cancer-targeted toxicity. Cytokine Growth Factor Rev. 2010;21(5):381-91.

6. Menezes M E, Bhatia S, Bhoopathi P, Das S K, Emdad L, Dasgupta S, et al.MDA-7/IL-24: multifunctional cancer killing cytokine Advances exper med biol. 2014;818:127-53.

7. Fisher P B, Gopalkrishnan R V, Chada S, Ramesh R, Grimm E A, Rosenfeld M R, et al. mda-7/IL-24, a novel cancer selective apoptosis inducing cytokine gene: from the laboratory into the clinic. Cancer Biol Ther. 2003;2(4 Suppl 1):S23-37.

8. Cunningham C C, Chada S, Merritt J A, Tong A, Senzer N, Zhang Y, et al. Clinical and local biological effects of an intratumoral injection of mda-7 (IL24; INGN 241) in patients with advanced carcinoma: a phase I study. Molecular therapy : the journal of the American Society of Gene Therapy 2005;11(1):149-59.

9. Nishikawa T, Ramesh R, Munshi A, Chada S, Meyn R E. Adenovirus-mediated mda-7 (IL24) gene therapy suppresses angiogenesis and sensitizes NSCLC xenograft tumors to radiation. Molecular therapy : the journal of the American Society of Gene Therapy 2004;9(6):818-28.

10. Ramesh R, Mhashilkar A M, Tanaka F, Saito Y, Branch C D, Sieger K, et al. Melanoma differentiation-associated gene 7/interleukin (IL)-24 is a novel ligand that regulates angiogenesis via the IL-22 receptor. Cancer research 2003;63(16):5105-13.

11. Sauane M, Su Z Z, Gupta P, Lebedeva I V, Dent P, Sarkar D, et al. Autocrine regulation of mda-7/IL-24 mediates cancer-specific apoptosis. Proceedings of the National Academy of Sciences of the United States of America 2008;105(28):9763-8.

12. Gupta P, Walter M R, Su Z Z, Lebedeva I V, Emdad L, Randolph A, et al. BiP/GRP78 is an intracellular target for MDA-7/IL-24 induction of cancer-specific apoptosis. Cancer research 2006;66(16):8182-91.

13. Fisher P B. Is mda-7/IL-24 a “magic bullet” for cancer? Cancer research 2005;65(22):10128-38.

14. Zhao Y, Ransom J F, Li A, Vedantham V, von Drehle M, Muth A N, et al. Dysregulation of cardiogenesis, cardiac conduction, and cell cycle in mice lacking miRNA-1-2. Cell 2007;129(2):303-17.

15. Gu S, Jin L, Zhang F, Sarnow P, Kay M A. Biological basis for restriction of microRNA targets to the 3′ untranslated region in mammalian mRNAs. Nature structural & molecular biology 2009;16(2):144-50.

16. Lytle J R, Yario T A, Steitz J A. Target mRNAs are repressed as efficiently by microRNA-binding sites in the 5′ UTR as in the 3′ UTR. Proceedings of the National Academy of Sciences of the United States of America 2007;104(23):9667-72.

17. Blower P E, Verducci J S, Lin S, Zhou J, Chung J H, Dai Z, et al. MicroRNA expression profiles for the NCI-60 cancer cell panel. Molecular cancer therapeutics 2007;6(5):1483-91.

18. Guo L, Xu J, Qi J, Zhang L, Wang J, Liang J, et al. MicroRNA-17-92a upregulation by estrogen leads to Bim targeting and inhibition of osteoblast apoptosis. Journal of cell science 2013;126(Pt 4):978-88.

19. le Sage C, Nagel R, Egan D A, Schrier M, Mesman E, Mangiola A, et al. Regulation of the p27(Kipl) tumor suppressor by miR-221 and miR-222 promotes cancer cell proliferation. The EMBO journal 2007;26(15):3699-708.

20. Zhang C Z, Zhang J X, Zhang A L, Shi Z D, Han L, Jia Z F, et al. MiR-221 and miR-222 target PUMA to induce cell survival in glioblastoma. Molecular cancer 2010;9:229.

21. Zhao J J, Lin J, Yang H, Kong W, He L, Ma X, et al. MicroRNA-221/222 negatively regulates estrogen receptor alpha and is associated with tamoxifen resistance in breast cancer. The Journal of biological chemistry 2008;283(45):31079-86.

22. Garofalo M, Di Leva G, Romano G, Nuovo G, Suh S S, Ngankeu A, et al. miR-221&222 regulate TRAIL resistance and enhance tumorigenicity through PTEN and TIMP3 downregulation. Cancer cell 2009;16(6):498-509.

23. Fornari F, Gramantieri L, Ferracin M, Veronese A, Sabbioni S, Calin G A, et al. MiR-221 controls CDKN1C/p57 and CDKN1B/p27 expression in human hepatocellular carcinoma. Oncogene 2008;27(43):5651-61.

24. Hamada N, Fujita Y, Kojima T, Kitamoto A, Akao Y, Nozawa Y, et al. MicroRNA expression profiling of NGF-treated PC12 cells revealed a critical role for miR-221 in neuronal differentiation. Neurochemistry international 2012;60(8):743-50.

25. Felicetti F, Errico M C, Bottero L, Segnalini P, Stoppacciaro A, Biffoni M, et al. The promyelocytic leukemia zinc finger-microRNA-221/-222 pathway controls melanoma progression through multiple oncogenic mechanisms. Cancer research 2008;68(8):2745-54.

26. Miracco C, Cosci E, Oliveri G, Luzi P, Pacenti L, Monciatti I, et al. Protein and mRNA expression of autophagy gene Beclin 1 in human brain tumours. International journal of oncology 2007;30(2):429-36.

27. Su Z, Shi Y, Friedman R, Qiao L, McKinstry R, Hinman D, et al. PEA3 sites within the progression elevated gene-3 (PEG-3) promoter and mitogen-activated protein kinase contribute to differential PEG-3 expression in Ha-ras and v-raf oncogene transformed rat embryo cells. Nucleic Acids Res 2001;29(8):1661-71.

28. Su Z, Emdad L, Sauane M, Lebedeva I V, Sarkar D, Gupta P, et al. Unique aspects of mda-7/IL-24 antitumor bystander activity: establishing a role for secretion of MDA-7/IL-24 protein by normal cells. Oncogene 2005;24(51):7552-66.

29. Dash R, Bhoopathi P, Das S K, Sarkar S, Emdad L, Dasgupta S, et al. Novel mechanism of MDA-7/IL-24 cancer-specific apoptosis through SARI induction. Cancer research 2014;74(2):563-74.

30. Wilson E N, Bristol M L, Di X, Maltese W A, Koterba K, Beckman M J, et al. A switch between cytoprotective and cytotoxic autophagy in the radiosensitization of breast tumor cells by chloroquine and vitamin D Hormones & cancer 2011;2(5):272-85.

31. Nassirpour R, Mehta P P, Baxi S M, Yin M J. miR-221 promotes tumorigenesis in human triple negative breast cancer cells. PloS one 2013;8(4):e62170.

32. Yacoub A, Hamed H A, Allegood J, Mitchell C, Spiegel S, Lesniak M S, et al. PERK-dependent regulation of ceramide synthase 6 and thioredoxin play a key role in mda-7/IL-24-induced killing of primary human glioblastoma multiforme cells. Cancer research 2010;70(3):1120-9.

33. Lebedeva I V, Su Z Z, Sarkar D, Kitada S, Dent P, Waxman S, et al. Melanoma differentiation associated gene-7, mda-7/interleukin-24, induces apoptosis in prostate cancer cells by promoting mitochondrial dysfunction and inducing reactive oxygen species. Cancer research 2003;63(23):8138-44.

34. Su M, Wang J, Wang C, Wang X, Dong W, Qiu W, et al. MicroRNA-221 inhibits autophagy and promotes heart failure by modulating the p27/CDK2/mTOR axis. Cell death and differentiation 2015;22(6):986-99.

35. Jung K H, Noh J H, Kim J K, Eun J W, Bae H J, Chang Y G, et al. Histone deacetylase 6 functions as a tumor suppressor by activating c-Jun NH2-terminal kinase-mediated beclin 1-dependent autophagic cell death in liver cancer. Hepatology 2012 ;56(2):644-57.

36. Bae H J, Jung K H, Eun J W, Shen Q, Kim H S, Park S J, et al. MicroRNA-221 governs tumor suppressor HDAC6 to potentiate malignant progression of liver cancer. J Hepatol. 2015; 63(2):408-19.

37. Bhutia S K, Dash R, Das S K, Azab B, Su Z Z, Lee S G, et al. Mechanism of autophagy to apoptosis switch triggered in prostate cancer cells by antitumor cytokine melanoma differentiation-associated gene 7/interleukin-24. Cancer research 2010;70(9):3667-76.

38. Xie Z G, Xie Y, Dong Q R. Inhibition of the mammalian target of rapamycin leads to autophagy activation and cell death of MG63 osteosarcoma cells. Oncology letters 2013;6(5):1465-69.

39. Thorburn A, Thamm D H, Gustafson D L. Autophagy and cancer therapy. Molecular pharmacology 2014;85(6):830-8.

40. Reed J C. Apoptosis-based therapies. Nature reviews Drug discovery 2002;1(2):111-21.

41. Tong A W, Nemunaitis J, Su D, Zhang Y, Cunningham C, Senzer N, et al. Intratumoral injection of INGN 241, a nonreplicating adenovector expressing the melanoma-differentiation associated gene-7 (mda-7/IL24): biologic outcome in advanced cancer patients. Molecular therapy : the journal of the American Society of Gene Therapy 2005;11(1):160-72.

42. Lebedeva I V, Su Z Z, Sarkar D, Gopalkrishnan R V, Waxman S, Yacoub A, et al. Induction of reactive oxygen species renders mutant and wild-type K-ras pancreatic carcinoma cells susceptible to Ad.mda-7-induced apoptosis. Oncogene 2005;24(4):585-96.

43. Lebedeva I V, Su Z Z, Emdad L, Kolomeyer A, Sarkar D, Kitada S, et al. Targeting inhibition of K-ras enhances Ad.mda-7-induced growth suppression and apoptosis in mutant K-ras colorectal cancer cells. Oncogene 2007;26(5):733-44.

44. Sarkar S, Azab B M, Das S K, Quinn B A, Shen X, Dash R, et al. Chemoprevention gene therapy (CGT): novel combinatorial approach for preventing and treating pancreatic cancer. Current molecular medicine 2013;13(7):1140-59.

45. Roldo C, Missiaglia E, Hagan J P, Falconi M, Capelli P, Bersani S, et al. MicroRNA expression abnormalities in pancreatic endocrine and acinar tumors are associated with distinctive pathologic features and clinical behavior. Journal of clinical oncology: official journal of the American Society of Clinical Oncology 2006;24(29):4677-84.

46. Pickford F, Masliah E, Britschgi M, Lucin K, Narasimhan R, Jaeger P A, et al. The autophagy-related protein beclin 1 shows reduced expression in early Alzheimer disease and regulates amyloid beta accumulation in mice. The Journal of clinical investigation 2008;118(6):2190-9.

47. Diskin T, Tal-Or P, Erlich S, Mizrachy L, Alexandrovich A, Shohami E, et al. Closed head injury induces upregulation of Beclin 1 at the cortical site of injury. J Neurotrauma. 2005;22(7):750-62.

48. Li D D, Wang L L, Deng R, Tang J, Shen Y, Guo J F, et al. The pivotal role of c-Jun NH2-terminal kinase-mediated Beclin 1 expression during anticancer agents-induced autophagy in cancer cells. Oncogene. 2009;28(6):886-98.

49. Ding Z B, Shi Y H, Zhou J, Qiu S J, Xu Y, Dai Z, et al. Association of autophagy defect with a malignant phenotype and poor prognosis of hepatocellular carcinoma. Cancer research 2008;68(22):9167-75.

50. Sarkar S, Ravikumar B, Floto R A, Rubinsztein D C. Rapamycin and mTOR-independent autophagy inducers ameliorate toxicity of polyglutamine-expanded huntingtin and related proteinopathies. Cell death and differentiation 2009;16(1):46-56.

Example 3 reference:

M. Allen, B. Pratscher, C. Krepler, K. Frei, C. Schofer, H. Pehamberger, et al. Alternative splicing of IL-24 in melanocytes by deletion of exons 3 and 5. International journal of immunogenetics, 32 (2005), 375-378.

M. Allen, B. Pratscher, F. Roka, C. Krepler, V. Wacheck, C. Schofer, et al. Loss of novel mda-7 splice variant (mda-7s) expression is associated with metastatic melanoma. The Journal of investigative dermatology 123 (2004), 583-588.

A. Andoh, M. Shioya, A. Nishida, S. Bamba, T. Tsujikawa, S. Kim-Mitsuyama, et al. Expression of IL-24, an activator of the JAK1/STAT3/SOCS3 cascade, is enhanced in inflammatory bowel disease. Journal of Immunology 183 (2009), 687-695.

P. Bhoopathi, N. Lee, A. K. Pradhan, X. N. Shen, S. K. Das, D. Sarkar, et al. mda-7/IL-24 Induces Cell Death in Neuroblastoma through a Novel Mechanism Involving AIF and ATM. Cancer Research. 76 (2016), 3572-3582.

S. K. Bhutia, S. K. Das, B. Azab, M. E. Menezes, P. Dent, X. Y. Wang, et al. Targeting breast cancer-initiating/stem cells with melanoma differentiation-associated gene-7/interleukin-24. International Journal of Cancer 133 (2013), 2726-2736.

S. K. Bhutia, S. K. Das, T. P. Kegelman, B. Azab, R. Dash, Z. Z. Su, et al. mda-7/IL-24 differentially regulates soluble and nuclear clusterin in prostate cancer. Journal of Cellular Physiology 227 (2012), 1805-1813.

S. K. Bhutia, R. Dash, S. K. Das, B. Azab, Z. Z. Su, S. G Lee, et al. Mechanism of autophagy to apoptosis switch triggered in prostate cancer cells by antitumor cytokine melanoma differentiation-associated gene 7/interleukin-24. Cancer Research 70 (2010), 3667-3676.

S. K. Bhutia, S. Mukhopadhyay, N. Sinha, D. N. Das, P. K. Panda, S. K. Patra, et al. Autophagy: cancer's friend or foe? Advances in Cancer Research 118 (2013), 61-95.

K. Buzas, J. J. Oppenheim, O. M Zack Howard. Myeloid cells migrate in response to IL-24. Cytokine 55 (2011), 429-434.

E. G. Caudell, J. B. Mumm, N. Poindexter, S. Ekmekcioglu, A. M Mhashilkar, X. H. Yang, et al. The protein product of the tumor suppressor gene, melanoma differentiation-associated gene 7, exhibits immunostimulatory activity and is designated IL-24. Journal of Immunology 168 (2002), 6041-6046.

S. Chada, D. Bocangel, R. Ramesh, E. A. Grimm, J. B. Mumm, A. M. Mhashilkar, et al. mda-7/IL24 kills pancreatic cancer cells by inhibition of the Wnt/PI3K signaling pathways: identification of IL-20 receptor-mediated bystander activity against pancreatic cancer. Molecular Therapy 11 (2005), 724-733.

S. Chada, A. M. Mhashilkar, Y. Liu, T. Nishikawa, D. Bocangel, M. Zheng, et al. mda-7 gene transfer sensitizes breast carcinoma cells to chemotherapy, biologic therapies and radiotherapy: correlation with expression of bcl-2 family members. Cancer Gene Therapy 13 (2006), 490-502.

S. Chada, A. M. Mhashilkar, R. Ramesh, J. B. Mumm, R. B. Sutton, D. Bocangel, et al. Bystander activity of Ad-mda7: human MDA-7 protein kills melanoma cells via an IL-20 receptor-dependent but STAT3-independent mechanism. Molecular Therapy 10 (2004), 1085-1095.

S. Chada, R. B. Sutton, S. Ekmekcioglu, J. Ellerhorst, J. B. Mumm, W. W. Leitner, et al. MDA-7/IL-24 is a unique cytokine—tumor suppressor in the IL-10 family International Immunopharmacology 4 (2004), 649-667.

J. Chen, S. Chada, A. Mhashilkar, J. M. Miano. Tumor suppressor MDA-7/IL-24 selectively inhibits vascular smooth muscle cell growth and migration. Molecular Therapy 8 (2003), 220-229.

P. Conti, D. Kempuraj, S. Frydas, K. Kandere, W. Boucher, R. Letourneau, et al. IL-10 subfamily members: IL-19, IL-20, IL-22, IL-24 and IL-26 Immunology Letters 88 (2003), 171-174.

C. C. Cunningham, S. Chada, J. A. Merritt, A. Tong, N. Senzer, Y. Zhang, et al. Clinical and local biological effects of an intratumoral injection of mda-7 (IL24; INGN 241) in patients with advanced carcinoma: a phase I study. Molecular Therapy 11 (2005), 149-159.

R. Dash, P. Bhoopathi, S. K. Das, S. Sarkar, L. Emdad, S. Dasgupta, et al. Novel mechanism of MDA-7/IL-24 cancer-specific apoptosis through SARI induction. Cancer Research 74 (2014), 563-574.

R. Dash, S. K. Bhutia, B. Azab, Z. Z Su, B. A. Quinn, T. P. Kegelmen, et al. mda-7/IL-24: a unique member of the IL-10 gene family promoting cancer-targeted toxicity. Cytokine & Growth Factor Reviews 21 (2010), 381-391.

W. G. Deng, J. Kwon, S. Ekmekcioglu, N. J. Poindexter, E. A. Grimm. IL-24 gene transfer sensitizes melanoma cells to erlotinib through modulation of the Apaf-1 and Akt signaling pathways. Melanoma Research 21 (2011), 44-56.

P. Dent, A. Yacoub, H. A. Hamed, M. A. Park, R. Dash, S. K. Bhutia, et al. MDA-7/IL-24 as a cancer therapeutic: from bench to bedside. Anticancer Drugs 21 (2010), 725-731.

L. Dumoutier, C. Leemans, D. Lejeune, S. V. Kotenko, J. C. Renauld. Cutting edge: STAT activation by IL-19, IL-20 and mda-7 through IL-20 receptor complexes of two types. Journal of Immunology 167 (2001), 3545-3549.

S. Ekmekcioglu, J. Ellerhorst, A. M. Mhashilkar, A. A. Sahin, C. M. Read, V. G. Prieto, et al. Down-regulated melanoma differentiation associated gene (mda-7) expression in human melanomas. International Journal of Cancer 94 (2001), 54-59.

J. A. Ellerhorst, V. G. Prieto, S. Ekmekcioglu, L. Broemeling, S. Yekell, S. Chada, et al. Loss of MDA-7 expression with progression of melanoma. Journal of Clinical Oncology 20 (2002), 1069-1074.

L. Emdad, I. V. Lebedeva, Z. Z. Su, P. Gupta, M. Sauane, R. Dash, et al. Historical perspective and recent insights into our understanding of the molecular and biochemical basis of the antitumor properties of mda-7/IL-24. Cancer biology & therapy 8 (2009), 391-400.

C. E. Eyler, J. N. Rich. Survival of the fittest: cancer stem cells in therapeutic resistance and angiogenesis. Journal of clinical oncology : official journal of the American Society of Clinical Oncology 26 (2008), 2839-2845.

K. Fernald, M. Kurokawa. Evading apoptosis in cancer. Trends Cell Biology 23 (2013), 620-633.

V. Filippov, E. L. Schmidt, M. Filippova, P. J. Duerksen-Hughes. Splicing and splice factor SRp55 participate in the response to DNA damage by changing isoform ratios of target genes. Gene 420 (2008), 34-41.

P. B. Fisher. Is mda-7/IL-24 a “magic bullet” for cancer? Cancer Research 65 (2005), 10128-10138.

P. B. Fisher, R. V. Gopalkrishnan, S. Chada, R Ramesh, E. A. Grimm, M. R. Rosenfeld, et al. mda-7/IL-24, a novel cancer selective apoptosis inducing cytokine gene: from the laboratory into the clinic. Cancer biology & therapy 2 (2003), S23-37.

P. B. Fisher, D. Sarkar, I. V. Lebedeva, L. Emdad, P. Gupta, M. Sauane, et al. Melanoma differentiation associated gene-7/interleukin-24 (mda-7/IL-24): novel gene therapeutic for metastatic melanoma. Toxicology and Applied Pharmacology 224 (2007), 300-307.

G. Fonseca-Camarillo, J. Furuzawa-Carballeda, J. Granados, J. K. Yamamoto-Furusho. Expression of interleukin (IL)-19 and IL-24 in inflammatory bowel disease patients: a cross-sectional study. Clinical and Experimental Immunology 177 (2014), 64-75.

K. L. Fuson, M. Zheng, M. Craxton, A. Pataer, R. Ramesh, S. Chada, et al. Structural mapping of post-translational modifications in human interleukin-24: role of N-linked glycosylation and disulfide bonds in secretion and activity. Journal of Biological Chemistry 284 (2009), 30526-30533.

P. Gao, X. Sun, X. Chen, J. Subjeck, X. Y. Wang. Secretion of stress protein grp170 promotes immune-mediated inhibition of murine prostate tumor. Cancer Immunology Immunotherapy 58 (2009), 1319-1328.

P. Gao, X. Sun, X. Chen, Y. Wang, B. A. Foster, J. Subjeck, et al. Secretable chaperone Grp170 enhances therapeutic activity of a novel tumor suppressor, mda-7/IL-24. Cancer Research 68 (2008), 3890-3898.

B. Gopalan, A. Litvak, S. Sharma, A. M. Mhashilkar, S. Chada, R. Ramesh. Activation of the Fas-FasL signaling pathway by MDA-7/IL-24 kills human ovarian cancer cells. Cancer Research 65 (2005), 3017-3024.

R. V. Gopalkrishnan, M. Sauane, P. B. Fisher. Cytokine and tumor cell apoptosis inducing activity of mda-7/IL-24. International Immunopharmacology 4 (2004), 635-647.

A. Greco, A. Di Benedetto, C. M. Howard, S. Kelly, R. Nande, Y. Dementieva, et al. Eradication of therapy-resistant human prostate tumors using an ultrasound-guided site-specific cancer terminator virus delivery approach. Molecular Therapy 18 (2010), 295-306.

J. Green, R. Nusse, R. van Amerongen. The role of Ryk and Ror receptor tyrosine kinases in Wnt signal transduction. Cold Spring Harbor perspectives in biology 6 (2014).

P. Gupta, Z. Z. Su, I. V. Lebedeva, D. Sarkar, M. Sauane, L. Emdad, et al. mda-7/IL-24: multifunctional cancer-specific apoptosis-inducing cytokine. Pharmacology and Therapeutics 111 (2006), 596-628.

P. Gupta, M. R. Walter, Z. Z. Su, I. V. Lebedeva, L. Emdad, A. Randolph, et al. BiP/GRP78 is an intracellular target for MDA-7/IL-24 induction of cancer-specific apoptosis. Cancer research 66 (2006a), 8182-8191.

D. Hanahan, R. A. Weinberg. The hallmarks of cancer. Cell 100 (2000), 57-70.

D. Hanahan, R. A. Weinberg. Hallmarks of cancer: the next generation. Cell 144 (2011), 646-674.

C. R. Hauck, D. A. Hsia, D. D. Schlaepfer. The focal adhesion kinase—a regulator of cell migration and invasion. IUBMB Life 53 (2002), 115-119.

M. He, P. Liang. IL-24 transgenic mice: in vivo evidence of overlapping functions for IL-20, IL-22, and IL-24 in the epidermis. Journal of Immunolgy 184 (2010), 1793-1798.

E. Y. Huang, M. T. Madireddi, R. V. Gopalkrishnan, M. Leszczyniecka, Z. Z. Su, I. V. Lebedeva, et al. Genomic structure, chromosomal localization and expression profile of a novel melanoma differentiation associated (mda-7) gene with cancer specific growth suppressing and apoptosis inducing properties. Oncogene 20 (2001), 7051-7063.

W. Huo, Z. M. Li, X. M. Zhu, Y. M. Bao, L. J. An. MDA-7/IL-24 suppresses tumor adhesion and invasive potential in hepatocellular carcinoma cell lines. Oncology Reports 30 (2013), 986-992.

H. Jiang, P. B. Fisher. Use of a sensitive and efficient subtraction hybridization protocol for the identification of genes differentially regulated during the induction of differentiation in human melanoma cells. Molecular Cellular Differentiation 1 (3) (1993): 285-299.

H. Jiang, J. J. Lin, Z. Z. Su, N. I. Goldstein, P. B. Fisher. Subtraction hybridization identifies a novel melanoma differentiation associated gene, mda-7, modulated during human melanoma differentiation, growth and progression. Oncogene 11 (1995), 2477-2486.

H. Jiang, Z. Z. Su, J. J. Lin, N. I. Goldstein, C. S. Young, P. B. Fisher. The melanoma differentiation associated gene mda-7 suppresses cancer cell growth. Proceedings of the National Academy of Sciences of the United States of America 93 (1996), 9160-9165.

S. H. Jin, D. Choi, Y. J. Chun, M. Noh. Keratinocyte-derived IL-24 plays a role in the positive feedback regulation of epidermal inflammation in response to environmental and endogenous toxic stressors. Toxicology and Applied Pharmacology 280 (2014), 199-206.

J. Karar, A. Maity. PI3K/AKT/mTOR Pathway in Angiogenesis. Frontiers in Molecular Neuroscience 4 (2011), 51.

T. W. Kragstrup, K. Otkjaer, C. Holm, A. Jorgensen, M. Hokland, L. Iversen, et al. The expression of IL-20 and IL-24 and their shared receptors are increased in rheumatoid arthritis and spondyloarthropathy. Cytokine 41 (2008), 16-23.

N. P. Kumar, V. V. Banurekha, D. Nair, P. Kumaran, C. K. Dolla, S. Babu. Type 2 diabetes—Tuberculosis co-morbidity is associated with diminished circulating levels of IL-20 subfamily of cytokines. Tuberculosis (Edinb) 95 (2015), 707-712.

S. Kumari, M. C. Bonnet, M. H. Ulvmar, K. Wolk, N. Karagianni, E. Witte, et al. Tumor necrosis factor receptor signaling in keratinocytes triggers interleukin-24-dependent psoriasis-like skin inflammation in mice. Immunity 39 (2013), 899-911.

S. Kunz, K. Wolk, E. Witte, K. Witte, W. D. Doecke, H. D. Volk, et al. Interleukin (IL)-19, IL-20 and IL-24 are produced by and act on keratinocytes and are distinct from classical ILs. Experimental dermatology 15 (2006), 991-1004.

I. V. Lebedeva, L. Emdad, Z. Z. Su, P. Gupta, M. Sauane, D. Sarkar, et al. mda-7/IL-24, novel anticancer cytokine: focus on bystander antitumor, radiosensitization and antiangiogenic properties and overview of the phase I clinical experience. International Journal of Oncology 31 (2007), 985-1007.

I. V. Lebedeva, M. Sauane, R. V. Gopalkrishnan, D. Sarkar, Z. Z. Su, P. Gupta, et al. mda-7/IL-24: exploiting cancer's Achilles' heel. Molecular Therapy 11 (2005), 4-18.

I. V. Lebedeva, D. Sarkar, Z.-z. Su, S. Kitada, P. Dent, C. A. Stein, et al. Bcl-2 and Bcl-xL differentially protect human prostate cancer cells from induction of apoptosis by melanoma differentiation associated gene-7, mda-7/IL-24. Oncogene 22 (2003b), 8758-8773.

I. V. Lebedeva, Z.-z. Su, Y. Chang, S. Kitada, J. C. Reed, P. B. Fisher. The cancer growth suppressing gene mda-7 induces apoptosis selectively in human melanoma cells. Oncogene 21 (2002), 708-718.

I. V. Lebedeva, Z. Z. Su, D. Sarkar, P. B. Fisher. Restoring apoptosis as a strategy for cancer gene therapy: focus on p53 and mda-7. Seminars in Cancer Biology 13 (2003a), 169-178.

I. V. Lebedeva, Z. Z. Su, D. Sarkar, R. V. Gopalkrishnan, S. Waxman, A. Yacoub, et al. Induction of reactive oxygen species renders mutant and wild-type K-ras pancreatic carcinoma cells susceptible to Ad.mda-7-induced apoptosis. Oncogene 24 (2005), 585-596.

I. V. Lebedeva, Z. Z. Su, D. Sarkar, S. Kitada, P. Dent, S. Waxman, et al.Melanoma differentiation associated gene-7, mda-7/interleukin-24, induces apoptosis in prostate cancer cells by promoting mitochondrial dysfunction and inducing reactive oxygen species. Cancer Research 63 (2003c), 8138-8144.

K. M. Lee, H. A. Kang, M. Park, H. Y. Lee, M. J. Song, K. Ko, et al. Interleukin-24 suppresses the growth of vascular smooth muscle cells by inhibiting H(2)O(2)-induced reactive oxygen species production. Pharmacology 90 (2012), 332-341.

R. X. Leng, H. F. Pan, J. H. Tao, D. Q. Ye. IL-19, IL-20 and IL-24: potential therapeutic targets for autoimmune diseases. Expert opinion on therapeutic targets 15 (2011), 119-126.

J. Liang, R. L. Huang, Q. Huang, Z. Peng, P. H. Zhang, Z. W. Wu. Adenovirus-mediated human interleukin 24 (MDA-7/IL-24) selectively suppresses proliferation and induces apoptosis in keloid fibroblasts. Annals of Plastic Surgery 66 (2011), 660-666.

S. W. Lin, M. T. Lee, F. C. Ke, P. P. Lee, C. J. Huang, M. M. Ip, et al. TGFbetal stimulates the secretion of matrix metalloproteinase 2 (MMP2) and the invasive behavior in human ovarian cancer cells, which is suppressed by MMP inhibitor BB3103. Clinical and Experimental Metastasis 18 (2000), 493-499.

J. Liu, J. Debnath. The Evolving, Multifaceted Roles of Autophagy in Cancer. Advances in Cancer Research 130 (2016), 1-53.

C. Lv, Q. Su, Y. Liang, J. Hu, S. Yuan. Oncolytic vaccine virus harbouring the IL-24 gene suppresses the growth of lung cancer by inducing apoptosis. Biochemical and biophysical research communications 476 (2016), 21-28.

Q. Ma, B. Jin, Y. Zhang, Y. Shi, C. Zhang, D. Luo, et al. Secreted recombinant human IL-24 protein inhibits the proliferation of esophageal squamous cell carcinoma Eca-109 cells in vitro and in vivo. Oncology reports 35 (2016), 2681-2690.

Y. Ma, H. Chen, Q. Wang, F. Luo, J. Yan, X. L. Zhang IL-24 protects against Salmonella typhimurium infection by stimulating early neutrophil Thl cytokine production, which in turn activates CD8+ T cells. European journal of immunology 39 (2009), 3357-3368.

Y. Ma, H. D. Chen, Y. Wang, Q. Wang, Y. Li, Y. Zhao, et al. Interleukin 24 as a novel potential cytokine immunotherapy for the treatment of Mycobacterium tuberculosis infection. Microbes and Infection 13 (2011), 1099-1110.

Y. F. Ma, Y. Ren, C. J. Wu, X. H. Zhao, H. Xu, D. Z. Wu, et al. Interleukin (IL)-24 transforms the tumor microenvironment and induces anticancer immunity in a murine model of colon cancer. Molecular Immunology 75 (2016), 11-20.

G. Maarof, L. Bouchet-Delbos, H. Gary-Gouy, I. Durand-Gasselin, R. Krzysiek, A. Dalloul. Interleukin-24 inhibits the plasma cell differentiation program in human germinal center B cells. Blood 115 (2010), 1718-1726.

B. T. MacDonald, X. He. Frizzled and LRPS/6 receptors for Wnt/beta-catenin signaling. Cold Spring Harbor perspectives in biology 4 (2012).

M. E. Menezes, S. Bhatia, P. Bhoopathi, S. K. Das, L. Emdad, S. Dasgupta, et al. MDA-7/IL-24: multifunctional cancer killing cytokine. Advances in Experimental Medicine and Biology 818 (2014), 127-153.

M. E. Menezes, X. N. Shen, S. K. Das, L. Emdad, C. Guo, F. Yuan, et al. MDA-7/IL-24 functions as a tumor suppressor gene in vivo in transgenic mouse models of breast cancer. Oncotarget 6 (2015), 36928-36942.

A. M. Mhashilkar, A. L. Stewart, K. Sieger, H. Y. Yang, A. H. Khimani, I Ito, et al. 2003. MDA-7 negatively regulates the beta-catenin and PI3K signaling pathways in breast and lung tumor cells. Molecular Therapy 8 (2003), 207-219.

R. Miyahara, S. Banerjee, K. Kawano, C. Efferson, N. Tsuda, Y. Miyahara, et al. Melanoma differentiation-associated gene-7 (mda-7)/interleukin (IL)-24 induces anticancer immunity in a syngeneic murine model. Cancer Gene Therapy 13 (2006), 753-761.

B. J. Morrison, J. C. Morris, J. C. Steel. Lung cancer-initiating cells: a novel target for cancer therapy. Target Oncology 8 (2013), 159-172.

D. M. Mosser, X. Zhang. Interleukin-10: new perspectives on an old cytokine. Immunological Reviews 226 (2008), 205-218.

J. B. Mumm, S. Ekmekcioglu, N. J. Poindexter, S. Chada, E. A. Grimm Soluble human MDA-7/IL-24: characterization of the molecular form(s) inhibiting tumor growth and stimulating monocytes. Journal of Interferon & Cytokine Research 26 (2006), 877-886.

J. Nace, S. J. Fortunato, H. Maul, R. Menon. The expression pattern of two novel cytokines (IL-24 and IL-29) in human fetal membranes. Journal of Perinatal Medicine 38 (2010), 665-670.

P. Naik, J. Karrim, D. Hanahan. The rise and fall of apoptosis during multistage tumorigenesis: down-modulation contributes to tumor progression from angiogenic progenitors. Genes and Development 10 (1996), 2105-2116.

T. Nishikawa, R. Ramesh, A. Munshi, S. Chada, R. E. Meyn. Adenovirus-mediated mda-7 (IL24) gene therapy suppresses angiogenesis and sensitizes NSCLC xenograft tumors to radiation. Molecular Therapy 9 (2004), 818-828.

J. Panneerselvam, J. Jin, M. Shanker, J. Lauderdale, J. Bates, Q Wang, et al. IL-24 inhibits lung cancer cell migration and invasion by disrupting the SDF-1/CXCR4 signaling axis. PloS one 10 (2015), e0122439.

J. Panneerselvam, A. Munshi, R. Ramesh. Molecular targets and signaling pathways regulated by interleukin (IL)-24 in mediating its antitumor activities. Journal of Molecular Signaling 8(2013), 15.

M. A. Park, T. Walker, A. P. Martin, J. Allegood, N. Vozhilla, L. Emdad, et al. MDA-7/IL-24-induced cell killing in malignant renal carcinoma cells occurs by a ceramide/CD95/PERK-dependent mechanism. Molecular Cancer Therapeutics 8 (2009), 1280-1291.

M. A. Park, A. Yacoub, D. Sarkar, L. Emdad, M. Rahmani, S Spiegel, et al. PERK-dependent regulation of MDA-7/IL-24-induced autophagy in primary human glioma cells. Autophagy 4 (2008), 513-515.

J. Parrish-Novak, W. Xu, T. Brender, L. Yao, C. Jones, J. West, et al. Interleukins 19, 20, and 24 signal through two distinct receptor complexes. Differences in receptor-ligand interactions mediate unique biological functions. Journal of Biological Chemistry 277 (2002), 47517-47523.

M. Pasparakis, I. Haase, F. O. Nestle. Mechanisms regulating skin immunity and inflammation. Nature reviews. Immunology 14 (2014), 289-301.

A. Pataer, S. A. Vorburger, S. Chada, S. Balachandran, G. N. Barber, J. A. Roth, et al. Melanoma differentiation-associated gene-7 protein physically associates with the double-stranded RNA-activated protein kinase PKR. Molecular Therapy 11 (2005), 717-723.

D. S. Pei, Z. X. Yang, B. F. Zhang, X. X. Yin, L. T. Li, H. Z. Li, et al Enhanced apoptosis-inducing function of MDA-7/IL-24 RGD mutant via the increased adhesion to tumor cells. Journal of Interferon & Cytokine Research 32 (2012), 66-73.

L. Persaud, D. De Jesus, O. Brannigan, M. Richiez-Paredes, J. Huaman, G. Alvarado, et al. Mechanism of Action and Applications of Interleukin 24 in Immunotherapy. International Journal of Molecular Sciences 17 (2016).

N. J. Poindexter, R. R. Williams, G. Powis, E. Jen, A. S Caudle, S. Chada, et al. IL-24 is expressed during wound repair and inhibits TGFalpha-induced migration and proliferation of keratinocytes. Experimental Dermatology 19 (2010), 714-722.

A. K. Pradhan, S. Talukdar, P. Bhoopathi, X. N. Shen, L. Emdad, S. K. Das, et al. mda-7/IL-24 Mediates Cancer Cell-Specific Death via Regulation of miR-221 and the Beclin-1 Axis. Cancer Research 77 (2017), 949-959.

M. Rahmani, M. Mayo, R. Dash, U. K. Sokhi, I. P. Dmitriev, D. Sarkar, et al. Melanoma differentiation associated gene-7/interleukin-24 potently induces apoptosis in human myeloid leukemia cells through a process regulated by endoplasmic reticulum stress. Molecular pharmacology 78 (2010), 1096-1104.

R. Ramesh, I. Ito, B. Gopalan, Y. Saito, A. M. Mhashilkar, S. Chada. Ectopic production of MDA-7/IL-24 inhibits invasion and migration of human lung cancer cells. Molecular Therapy 9 (2004), 510-518.

J. Romer, E. Hasselager, P. J. Norby, T. Steiniche, J. Thorn Clausen, K. Kragballe. Epidermal overexpression of interleukin-19 and -20 mRNA in psoriatic skin disappears after short-term treatment with cyclosporine a or calcipotriol. Journal of Investigative Dermatology 121 (2003), 1306-1311.

A. Sahoo, C. G. Lee, A. Jash, J. S. Son, G. Kim, H. K. Kwon, et al. Stat6 and c-Jun mediate Th2 cell-specific IL-24 gene expression. Journal of Immunology 186 (2011), 4098-4109.

S. Sano, K. S. Chan, S. Carbaj al, J. Clifford, M. Peavey, K. Kiguchi, et al. Stat3 links activated keratinocytes and immunocytes required for development of psoriasis in a novel transgenic mouse model. Nature Medicine 11 (2005), 43-49.

D. Sarkar, I. V. Lebedeva, P. Gupta, L. Emdad, M. Sauane, P. Dent, et al. Melanoma differentiation associated gene-7 (mda-7)/IL-24: a ‘magic bullet’ for cancer therapy? Expert Opinion on Biological Therapy 7 (2007), 577-586.

D. Sarkar, Z. Z. Su, I. V. Lebedeva, M. Sauane, R. V. Gopalkrishnan, P. Dent, et al. mda-7 (IL-24): signaling and functional roles. BioTechniques Supplement (2002), 30-39.

D. Sarkar, Z. Z. Su, I. V. Lebedeva, M. Sauane, R. V. Gopalkrishnan, K. Valerie, et al. mda-7 (IL-24) Mediates selective apoptosis in human melanoma cells by inducing the coordinated overexpression of the GADD family of genes by means of p38 MAPK. Proceedings of the National Academy of Sciences of the United States of America 99 (2002), 10054-10059.

D. Sarkar, Z. Z. Su, E. S. Park, N. Vozhilla, P. Dent, D. T. Curiel, et al. A cancer terminator virus eradicates both primary and distant human melanomas. Cancer gene therapy 15 (2008), 293-302.

D. Sarkar, Z. Z. Su, N. Vozhilla, E. S. Park, P. Gupta, P. B. Fisher. Dual cancer-specific targeting strategy cures primary and distant breast carcinomas in nude mice. Proceedings of the National Academy of Sciences of the United States of America 102 (2005), 14034-14039.

S. Sarkar, B. A. Quinn, X. Shen, P. Dent, S. K. Das, L. Emdad, et al. Reversing translational suppression and induction of toxicity in pancreatic cancer cells using a chemoprevention gene therapy approach. Molecular pharmacology 87 (2015), 286-295.

M. Sauane, R. V. Gopalkrishnan, I. Lebedeva, M. X. Mei, D. Sarkar, Z. Z. Su, et al. Mda-7/IL-24 induces apoptosis of diverse cancer cell lines through JAK/STAT-independent pathways. Journal of cellular physiology 196 (2003), 334-345.

M. Sauane, R. V. Gopalkrishnan, H.-t. Choo, P. Gupta, I. V. Lebedeva, A. Yacoub, et al. Mechanistic aspects of mda-7/IL-24 cancer cell selectivity analyzed via a bacterial fusion protein. Oncogene 23 (2004), 7679-7690.

M. Sauane, R. V. Gopalkrishnan, D. Sarkar, Z. Z. Su, I. V. Lebedeva, P. Dent, et al. MDA-7/IL-24: novel cancer growth suppressing and apoptosis inducing cytokine. Cytokine & growth factor reviews 14 (2003), 35-51.

M. Sauane, P. Gupta, I. V. Lebedeva, Z. Z. Su, D. Sarkar, A. Randolph, et al. N-glycosylation of MDA-7/IL-24 is dispensable for tumor cell-specific apoptosis and “bystander” antitumor activity. Cancer Research 66 (2006), 11869-11877.

M. Sauane, Z. Z. Su, P. Gupta, I. V. Lebedeva, P. Dent, D. Sarkar, et al. Autocrine regulation of mda-7/IL-24 mediates cancer-specific apoptosis. Proceedings of the National Academy of Sciences of the United States of America 105 (2008), 9763-9768.

G. Schaefer, C. Venkataraman, U. Schindler. Cutting edge: FISP (IL-4-induced secreted protein), a novel cytokine-like molecule secreted by Th2 cells. Journal of Immunology 166 (2001), 5859-5863.

R. K. Seong, Y. K. Choi, O. S. Shin. MDA7/IL-24 is an anti-viral factor that inhibits influenza virus replication. Journal of Microbiology 54 (2016), 695-700.

B. A. Shapiro, N. T. Vu, M. D. Shultz, J. C. Chultz, J. A. Mietla, M. M. Gouda, et al. Melanoma differentiation associated gene 7/IL-24 exerts cytotoxic effects by altering the alternative splicing of Bcl-x pre-mRNA via he SRC/PKC□ signaling axis. J. Biol. Chem., 291 (2017), 21669-21681.

K. A. Sieger, A. M. Mhashilkar, A. Stewart, R. B. Sutton, R. W. Strube, S. Y. Chen, et al. The tumor suppressor activity of MDA-7/IL-24 is mediated by intracellular protein expression in NSCLC cells. Molecular Therapy 9 (2004), 355-367.

Z. Z. Su, L. Emdad, M. Sauane, I. V. Lebedeva, D. Sarkar, P. Gupta, et al. Unique aspects of mda-7/IL-24 antitumor bystander activity: establishing a role for secretion of MDA-7/IL-24 protein by normal cells. Oncogene 24 (2005), 7552-7566.

Z.-z. Su, I. V. Lebedeva, D. Sarkar, R. V. Gopalkrishnan, M. Sauane, C. Sigmon, et al. Melanoma differentiation associated gene-7, mda-7/IL-24, selectively induces growth suppression, apoptosis and radiosensitization in malignant gliomas in a p53-independent manner. Oncogene 22 (2003), 1164 -1180.

Z. Z. Su, M. T. Madireddi, J. J. Lin, C. S. Young, S. Kitada, J. C. Reed, et al. The cancer growth suppressor gene mda-7 selectively induces apoptosis in human breast cancer cells and inhibits tumor growth in nude mice. Proceedings of the National Academy of Sciences of the United States of America 95 (1998), 14400-14405.

S. Talukdar, L. Emdad, S. K. Das, D. Sarkar, P. B. Fisher. Evolving Strategies for Therapeutically Targeting Cancer Stem Cells. Advances in Cancer Research 131 (2016), 159-191.

H. Tamai, K. Miyake, H. Yamaguchi, M. Takatori, K. Dan, K. Inokuchi, et al. AAV8 vector expressing IL24 efficiently suppresses tumor growth mediated by specific mechanisms in MLL/AF4-positive ALL model mice. Blood 119 (2012), 64-71.

H. Tian, L. Li, B. Zhang, J. Di, F. Chen, H. Li, et al. Critical role of lysine 123 in the ubiquitin-mediated degradation of MDA-7/IL-24. Journal of Interferon & Cytokine Research 32 (2012), 575-582.

A. W. Tong, J. Nemunaitis, D. Su, Y. Zhang, C. Cunningham, N. Senzer, et al. Intratumoral injection of INGN 241, a nonreplicating adenovector expressing the melanoma-differentiation associated gene-7 (mda-7/IL24): biologic outcome in advanced cancer patients. Molecular Therapy 11 (2005), 160-172.

G. Vargas-Alarcon, C. Posadas-Romero, T. Villarreal-Molina, E. Alvarez-Leon, J. Angeles-Martinez, R. Posadas-Sanchez, et al. IL-24 gene polymorphisms are associated with cardiometabolic parameters and cardiovascular risk factors but not with premature coronary artery disease: the genetics of atherosclerotic disease Mexican study. Journal of Interferon & Cytokine Research 34 (2014), 659-666.

M. Wang, P. Liang. Interleukin-24 and its receptors. Immunology 114 (2005), 166-170.

M. Wang, Z. Tan, R. Zhang, S. V. Kotenko, P. Liang. Interleukin 24 (MDA-7/MOB-5) signals through two heterodimeric receptors, IL-22R1/IL-20R2 and IL-20R1/IL-20R2. Journal of Biological Chemistry 277 (2002), 7341-7347.

X. Wang, Z. Ye, J. Zhong, J. Xiang, J. Yang. Adenovirus-mediated 11-24 expression suppresses hepatocellular carcinoma growth via induction of cell apoptosis and cycling arrest and reduction of angiogenesis. Cancer Biotherapy & Radiopharmaceuticals 22 (2007), 56-63.

Y. H. Wang, D. T. Scadden. Harnessing the apoptotic programs in cancer stem-like cells. EMBO reports 16 (2015), 1084-1098.

Z. Wang, Y. Wang, Y. Chen, J. Lv. The IL-24 gene protects human umbilical vein endothelial cells against H(2)O(2)-induced injury and may be useful as a treatment for cardiovascular disease. International Journal of Molecular Medicine 37 (2016), 581-592.

B. Weiss, K. Wolk, B. H. Grunberg, H. D. Volk, W. Sterry, K. Asadullah, et al. Cloning of murine IL-22 receptor alpha 2 and comparison with its human counterpart. Genes Immunology 5 (2004), 330-336.

R. Weiss, J. Laengle, M. Sachet, A.P. Shurygina, O. Kiselev, A. Egorov, et al. Interleukin-24 inhibits influenza A virus replication in vitro through induction of toll-like receptor 3 dependent apoptosis. Antiviral Research 123 (2015), 93-104.

D. R. Welch, P. B. Fisher. Preface. Adv. Cancer Research 132 (2016), xi-xiv.

E. L. Whitaker, V. Filippov, M. Filippova, C. F. Guerrero-Juarez, P. J. Duerksen-Hughes. Splice variants of mda-7/IL-24 differentially affect survival and induce apoptosis in U2OS cells. Cytokine 56 (2011), 272-281.

E. L. Whitaker, V. A. Filippov, P. J. Duerksen-Hughes. Interleukin 24: mechanisms and therapeutic potential of an anti-cancer gene. Cytokine and Growth Factor Reviews 23 (2012), 323-331.

K. Wolk, H. S. Haugen, W. Xu, E. Witte, K. Waggie, M. Anderson, et al. IL-22 and IL-20 are key mediators of the epidermal alterations in psoriasis while IL-17 and IFN-gamma are not. Journal of Molecular Medicine (Berl) 87 (2009), 523-536.

K. Wolk, S. Kunz, E. Witte, M. Friedrich, K. Asadullah, R. Sabat. IL-22 increases the innate immunity of tissues. Immunity 21 (2004), 241-254.

Z. Xu, A. M. Robitaille, J. D. Berndt, K. C. Davidson, K. A. Fischer, J. Mathieu, et al. Wnt/beta-catenin signaling promotes self-renewal and inhibits the primed state transition in naive human embryonic stem cells. Proceedings of the National Academy of Sciences of the United States of America 113 (2016), E6382-E6390.

M. Zheng, D. Bocangel, B. Doneske, A. Mhashilkar, R. Ramesh, K. K. Hunt, et al. Human interleukin 24 (MDA-7/IL-24) protein kills breast cancer cells via the IL-20 receptor and is antagonized by IL-10. Cancer immunology, immunotherapy : CII 56 (2007), 205-215.

Y. Zheng, D. M. Danilenko, P. Valdez, I. Kasman, J. Eastham-Anderson, J. Wu, et al. Interleukin-22, a T(H)17 cytokine, mediates IL-23-induced dermal inflammation and acanthosis. Nature 445 (2007), 648-651.

B. Zhuo, Y. Shi, H. Qin, Q. Sun, Z. Li, F. Zhang, et al. Interleukin-24 inhibits osteosarcoma cell migration and invasion via the JNK/c-Jun signaling pathways. Oncology letters 13 (2017), 4505-4511.

### EMBODIMENTS

Embodiment 1. A method of detecting a miR-221 level in a cancer patient, wherein said cancer patient has received a MDA-7 treatment, said method comprising:
(i) obtaining a post-treatment biological sample from said cancer patient; and
(ii) detecting a post-treatment level of miR-221 in said post-treatment biological sample.

Embodiment 2. The method of embodiment 1, wherein said post-treatment biological sample is a tumor biopsy.

Embodiment 3. The method of embodiment 1 or 2, wherein said post-treatment biological sample comprises a circulating tumor cell.

4. The method of any one of embodiments 1-3, wherein said detecting comprises performing real-time PCR.

5. The method of any one of embodiments 1-3, wherein said detecting comprises performing in situ hybridization.

Embodiment 6. The method of any one of embodiments 1-5, further comprising detecting a post-treatment level of beclin-1 in said post-treatment biological sample.

Embodiment 7. The method of embodiment 6, wherein said detecting comprises performing real-time PCR.

Embodiment 8. The method of embodiment 6, wherein said detecting comprises performing Western blotting analysis.

Embodiment 9. The method of any one of embodiments 1-8, wherein said detecting a post-treatment level of miR-221 comprises detecting a post-treatment level of MMP, a post-treatment level of TIMP3, a post-treatment level of BMP2, or a post-treatment level of secreted uPAR isoform2 in said post-treatment biological sample.

Embodiment 10. The method of any one of embodiments 1-9, further comprising:
(i) obtaining a pre-treatment biological sample from said cancer patient prior to said cancer patient receiving a MDA-7 treatment; and
(ii) detecting a pre-treatment level of miR-221 in said pre-treatment biological sample.

Embodiment 11. The method of embodiment 10, wherein said pre-treatment biological sample is a tumor biopsy.

Embodiment 12. The method of embodiment 10 or 11, wherein said pre-treatment biological sample comprises a circulating tumor cell.

Embodiment 13. The method of any one of embodiments 10-12, wherein said detecting comprises performing real-time PCR.

Embodiment 14. The method of any one of embodiments 10-12, wherein said detecting comprises performing in situ hybridization.

Embodiment 15. The method of any one of embodiments 1-14, wherein said post-treatment level of miR-221 detected in said post-treatment biological sample is compared to said pre-treatment level of miR-221 detected in said pre-treatment biological sample.

Embodiment 16. The method of one of embodiments 10-15, wherein said detecting a pre-treatment level of miR-221 comprises detecting a pre-treatment level of MMP, a pre-treatment level of TIMP3, a pre-treatment level of BMP2, or a pre-treatment level of secreted uPAR isoform2 in said pre-treatment biological sample.

Embodiment 17. The method of embodiment 16, wherein said post-treatment level of MMP detected in said post-treatment biological sample is compared to said pre-treatment level of MMP in said pre-treatment biological sample.

Embodiment 18. The method of embodiment 16, wherein said post-treatment level of TIMP3 detected in said post-treatment biological sample is compared to said pre-treatment level of TIMP3 in said pre-treatment biological sample.

Embodiment 19. The method of embodiment 16, wherein said post-treatment level of BMP2 detected in said post-treatment biological sample is compared to said pre-treatment level of BMP2 in said pre-treatment biological sample.

Embodiment 20. The method of embodiment 16, wherein said post-treatment level of secreted uPAR isoform2 detected in said post-treatment biological sample is compared to said pre-treatment level of secreted uPAR isoform2 in said pre-treatment biological sample.

Embodiment 21. The method of any one of embodiments 1-20, wherein said cancer patient has been further treated with an additional anti-cancer agent.

Embodiment 22. The method of embodiment 21, wherein said additional anti-cancer agent is a ROS inducer.

Embodiment 23. The method of any one of embodiments 1-22, wherein said cancer patient has melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer.

Embodiment 24. The method of one of embodiments 1-23, wherein said cancer patient being treated has a metastatic cancer.

Embodiment 25. A method of detecting a beclin-1 level in a cancer patient, wherein said cancer patient has received a MDA-7 treatment, said method comprising:
(i) obtaining a post-treatment biological sample from said cancer patient; and
(ii) detecting a post-treatment level of beclin-1 in said post-treatment biological sample.

Embodiment 26. The method of embodiment 25, wherein said post-treatment biological sample is a tumor biopsy.

Embodiment 27. The method of any one of embodiments 25-26, wherein said post-treatment biological sample comprises a circulating tumor cell.

Embodiment 28. The method of any one of embodiments 25-27, wherein said detecting comprises performing real-time PCR.

Embodiment 29. The method of any one of embodiments 25-27, wherein said detecting comprises performing Western blotting analysis.

Embodiment 30. The method of any one of embodiments 25-29, further comprising detecting a post-treatment level of miR-221 in said post-treatment biological sample.

Embodiment 31. The method of embodiment 30, wherein said detecting comprises performing real-time PCR.

Embodiment 32. The method of embodiment 30, wherein said detecting comprises performing in situ hybridization.

Embodiment 33. The method of any one of embodiments 25-32, wherein said detecting a post-treatment level of beclin-1 comprises detecting a post-treatment level of MMP, a post-treatment level of TIMP3, a post-treatment level of BMP2, or a post-treatment level of secreted uPAR isoform2 in said post-treatment biological sample.

Embodiment 34. The method of any one of embodiments 25-33, further comprising:
(i) obtaining a pre-treatment biological sample from said cancer patient prior to said cancer patient receiving a MDA-7 treatment; and
(ii) detecting a pre-treatment level of beclin-1 in said pre-treatment biological sample.

Embodiment 35. The method of embodiment 34, wherein said pre-treatment biological sample is a tumor biopsy.

36. The method of embodiment 34 or 35, wherein said pre-treatment biological sample comprises a circulating tumor cell.

Embodiment 37. The method of any one of embodiments 34-36, wherein said detecting comprises performing real-time PCR.

Embodiment 38. The method of any one of embodiments 34-36, wherein said detecting comprises performing Western blotting analysis.

Embodiment 39. The method of any one of embodiments 25-38, wherein said post-treatment level of beclin-1 detected in said post-treatment biological sample is compared to said pre-treatment level of beclin-1 detected in said pre-treatment biological sample.

Embodiment 40. The method of one of embodiments 25-39, wherein said detecting a pre-treatment level of beclin-1 comprises detecting a pre-treatment level of MMP, a pre-treatment level of TIMP3, a pre-treatment level of BMP2, or a pre-treatment level of secreted uPAR isoform2 in said pre-treatment biological sample.

Embodiment 41. The method of embodiment 40, wherein said post-treatment level of MMP detected in said post-treatment biological sample is compared to said pre-treatment level of MMP in said pre-treatment biological sample.

Embodiment 42. The method of embodiment 40, wherein said post-treatment level of TIMP3 detected in said post-treatment biological sample is compared to said pre-treatment level of TIMP3 in said pre-treatment biological sample.

Embodiment 43. The method of embodiment 40, wherein said post-treatment level of BMP2 detected in said post-treatment biological sample is compared to said pre-treatment level of BMP2 in said pre-treatment biological sample.

Embodiment 44. The method of embodiment 40, wherein said post-treatment level of secreted uPAR isoform2 detected in said post-treatment biological sample is compared to said pre-treatment level of secreted uPAR isoform2 in said pre-treatment biological sample.

Embodiment 45. The method of any one of embodiments 25-44, wherein said cancer patient has been further treated with an additional anti-cancer agent.

Embodiment 46. The method of embodiment 45, wherein said additional anti-cancer agent is a ROS inducer.

Embodiment 47. The method of any one of embodiments 25-46, wherein said cancer patient has melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer.

Embodiment 48. The method of one of embodiments 25-47, wherein said cancer patient being treated has a metastatic cancer.

Embodiment 49. A method of treating cancer in a subject in need thereof, wherein said subject has a cancer expressing miR-221 and not expressing MDA-7, said method comprising administering to said subject an effective amount of MDA-7.

Embodiment 50. The method of embodiment 49 wherein said cancer does not express beclin-1.

Embodiment 51. The method of any one of embodiments 49-50, further comprising, prior to administering said effective amount of MDA-7:
(i) obtaining a pre-treatment biological sample from said subject; and
(ii) detecting a pre-treatment level of miR-221 in said pre-treatment biological sample.

Embodiment 52. The method of embodiment 51, wherein said pre-treatment biological sample is a tumor biopsy.

Embodiment 53. The method of one of embodiments 51-52, wherein said pre-treatment biological sample comprises a circulating tumor cell.

Embodiment 54. The method of any one of embodiments 51-53, wherein said detecting comprises performing real-time PCR.

55. The method of any one of embodiments 51-53, wherein said detecting comprises performing in situ hybridization.

Embodiment 56. The method of any one of embodiments 51-55, wherein said pre-treatment level of miR-221 in said pre-treatment biological sample is compared against a standard control.

Embodiment 57. The method of any one of embodiments 51-55, wherein said detecting a pre-treatment level of miR-221 comprises detecting a pre-treatment level of MMP, a pre-treatment level of TIMP3, a pre-treatment level of BMP2, or a pre-treatment level of secreted uPAR isoform2 in said pre-treatment biological sample.

Embodiment 58. The method of embodiment 57, wherein said pre-treatment level of MMP in said pre-treatment biological sample is compared against a standard control.

Embodiment 59. The method of embodiment 57, wherein said pre-treatment level of TIMP3 in said pre-treatment biological sample is compared against a standard control.

Embodiment 60. The method of embodiment 57, wherein said pre-treatment level of BMP2 in said pre-treatment biological sample is compared against a standard control.

Embodiment 61. The method of embodiment 57, wherein said pre-treatment level of secreted uPAR isoform2 in said pre-treatment biological sample is compared against a standard control.

Embodiment 62. The method of any one of embodiments 49-61, wherein administering said effective amount of MDA-7 reverses a multidrug chemoresistance.

Embodiment 63. The method of any one of embodiments 49-62, further comprising administering to said subject an additional anti-cancer agent.

Embodiment 64. The method of embodiment 63, wherein said additional anti-cancer agent is a ROS inducer.

Embodiment 65. The method of any one of embodiments 49-64, wherein said cancer is melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer.

Embodiment 66. The method of any one of embodiments 49-65, wherein said cancer is a metastatic cancer.

Embodiment 67. A method of treating cancer in a subject in need thereof, wherein said subject has a cancer not expressing beclin-1 and not expressing MDA-7, said method comprising administering to said subject an effective amount of MDA-7.

Embodiment 68. The method of embodiment 67, wherein said cancer expresses miR-221.

Embodiment 69. The method of embodiment 67 or 68, further comprising, prior to administering said effective amount of MDA-7:
(i) obtaining a pre-treatment biological sample from said subject; and
(ii) detecting a pre-treatment level of beclin-1 in said pre-treatment biological sample.

Embodiment 70. The method of embodiment 69, wherein said pre-treatment biological sample is a tumor biopsy.

Embodiment 71. The method of embodiment 69 or 70, wherein said pre-treatment biological sample comprises a circulating tumor cell.

Embodiment 72. The method of any one of embodiments 69-71, wherein said detecting comprises performing real-time PCR.

Embodiment 73. The method of any one of embodiments 69-71, wherein said detecting comprises performing Western blotting analysis.

Embodiment 74. The method of any one of embodiments 69-73, wherein said pre-treatment level of beclin-1 in said pre-treatment biological sample is compared against a standard control.

Embodiment 75. The method of any one of embodiments 69-74, wherein said detecting a pre-treatment level of beclin-1 comprises detecting a pre-treatment level of MMP, a pre-treatment level of TIMP3, a pre-treatment level of BMP2, or a pre-treatment level of secreted uPAR isoform 2 in said pre-treatment biological sample.

Embodiment 76. The method of embodiment 75, wherein said pre-treatment level of MMP in said pre-treatment biological sample is compared against a standard control.

Embodiment 77. The method of embodiment 75, wherein said pre-treatment level of TIMP3 in said pre-treatment biological sample is compared against a standard control.

Embodiment 78. The method of embodiment 75, wherein said pre-treatment level of BMP2 in said pre-treatment biological sample is compared against a standard control.

Embodiment 79. The method of embodiment 75, wherein said pre-treatment level of secreted uPAR isoform2 in said pre-treatment biological sample is compared against a standard control.

Embodiment 80. The method of any one of embodiments 67-75, wherein administering said effective amount of MDA-7 reverses a multidrug chemoresistance.

Embodiment 81. The method of any one of embodiments 67-75, further comprising administering to said subject an additional anti-cancer agent.

Embodiment 82. The method of embodiment 81, wherein said additional anti-cancer agent is a ROS inducer.

Embodiment 83. The method of any one of embodiments 67-82, wherein said cancer is melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer.

Embodiment 84. The method of any one of embodiments 67-83, wherein said cancer is a metastatic cancer.

Embodiment 85. A method of inhibiting cancer-associated angiogenesis in a subject in need thereof, said method comprising administering to said subject an effective amount of MDA-7.

Embodiment 86. A method of treating an autoimmune disease in a subject in need thereof, said method comprising administering to said subject an effective amount of MDA-7.

Embodiment 87. A method of treating an infectious disease in a subject in need thereof, said method comprising administering to said subject an effective amount of MDA-7.

Embodiment 88. A method of treating an inflammatory disease in a subject in need thereof, said method comprising administering to said subject an effective amount of MDA-7.

Embodiment 89. A method of treating a cardiovascular disease in a subject in need thereof, said method comprising administering to said subject an effective amount of MDA-7.

What is claimed is:

1. A method of detecting a miR-221 level in a cancer patient, wherein said cancer patient has received a MDA-7 treatment, said method comprising:
(iii) obtaining a post-treatment biological sample from said cancer patient; and
(iv) detecting a post-treatment level of miR-221 in said post-treatment biological sample.

2. The method of claim 1, wherein said post-treatment biological sample is a tumor biopsy.

3. The method of claim 1, wherein said post-treatment biological sample comprises a circulating tumor cell.

4. The method of claim 1, wherein said detecting comprises performing real-time PCR.

5. The method of claim 1, wherein said detecting comprises performing in situ hybridization.

6. The method of claim 1, further comprising detecting a post-treatment level of beclin-1 in said post-treatment biological sample.

7. The method of claim 6, wherein said detecting comprises performing real-time PCR.

8. The method of claim 6, wherein said detecting comprises performing Western blotting analysis.

9. The method of claim 1, wherein said detecting a post-treatment level of miR-221 comprises detecting a post-treatment level of MMP, a post-treatment level of TIMP3, a post-treatment level of BMP2, or a post-treatment level of secreted uPAR isoform2 in said post-treatment biological sample.

10. The method of claim 1, further comprising:
(iii) obtaining a pre-treatment biological sample from said cancer patient prior to said cancer patient receiving a MDA-7 treatment; and
(iv) detecting a pre-treatment level of miR-221 in said pre-treatment biological sample.

11. The method of claim 10, wherein said pre-treatment biological sample is a tumor biopsy.

12. The method of claim 10, wherein said pre-treatment biological sample comprises a circulating tumor cell.

13. The method of claim 10, wherein said detecting comprises performing real-time PCR.

14. The method of claim 10, wherein said detecting comprises performing in situ hybridization.

15. The method of claim 1, wherein said post-treatment level of miR-221 detected in said post-treatment biological sample is compared to said pre-treatment level of miR-221 detected in said pre-treatment biological sample.

16. The method of claim 10, wherein said detecting a pre-treatment level of miR-221 comprises detecting a pre-treatment level of MMP, a pre-treatment level of TIMP3, a pre-treatment level of BMP2, or a pre-treatment level of secreted uPAR isoform2 in said pre-treatment biological sample.

17. The method of claim 16, wherein said post-treatment level of MMP detected in said post-treatment biological sample is compared to said pre-treatment level of MMP in said pre-treatment biological sample.

18. The method of claim 16, wherein said post-treatment level of TIMP3 detected in said post-treatment biological sample is compared to said pre-treatment level of TIMP3 in said pre-treatment biological sample.

19. The method of claim 16, wherein said post-treatment level of BMP2 detected in said post-treatment biological sample is compared to said pre-treatment level of BMP2 in said pre-treatment biological sample.

20. The method of claim 16, wherein said post-treatment level of secreted uPAR isoform2 detected in said post-treatment biological sample is compared to said pre-treatment level of secreted uPAR isoform2 in said pre-treatment biological sample.

21. The method of claim 1, wherein said cancer patient has been further treated with an additional anti-cancer agent.

22. The method of claim 21, wherein said additional anti-cancer agent is a ROS inducer.

23. The method of claim 1, wherein said cancer patient has melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer.

24. The method of claim 1, wherein said cancer patient being treated has a metastatic cancer.

25. A method of detecting a beclin-1 level in a cancer patient, wherein said cancer patient has received a MDA-7 treatment, said method comprising:
(iii) obtaining a post-treatment biological sample from said cancer patient; and
(iv) detecting a post-treatment level of beclin-1 in said post-treatment biological sample.

26. The method of claim 25, wherein said post-treatment biological sample is a tumor biopsy.

27. The method of claim 25, wherein said post-treatment biological sample comprises a circulating tumor cell.

28. The method of claim 25, wherein said detecting comprises performing real-time PCR.

29. The method of claim 25, wherein said detecting comprises performing Western blotting analysis.

30. The method of claim 25, further comprising detecting a post-treatment level of miR-221 in said post-treatment biological sample.

31. The method of claim 30, wherein said detecting comprises performing real-time PCR.

32. The method of claim 30, wherein said detecting comprises performing in situ hybridization.

33. The method of claim 25, wherein said detecting a post-treatment level of beclin-1 comprises detecting a post-treatment level of MMP, a post-treatment level of TIMP3, a post-treatment level of BMP2, or a post-treatment level of secreted uPAR isoform2 in said post-treatment biological sample.

34. The method of claim 25, further comprising:
(iii) obtaining a pre-treatment biological sample from said cancer patient prior to said cancer patient receiving a MDA-7 treatment; and
(iv) detecting a pre-treatment level of beclin-1 in said pre-treatment biological sample.

35. The method of claim 34, wherein said pre-treatment biological sample is a tumor biopsy.

36. The method of claim 34, wherein said pre-treatment biological sample comprises a circulating tumor cell.

37. The method of claim 34, wherein said detecting comprises performing real-time PCR.

38. The method of claim 34, wherein said detecting comprises performing Western blotting analysis.

39. The method of claim 25, wherein said post-treatment level of beclin-1 detected in said post-treatment biological sample is compared to said pre-treatment level of beclin-1 detected in said pre-treatment biological sample.

40. The method of claim 25, wherein said detecting a pre-treatment level of beclin-1 comprises detecting a pre-treatment level of MMP, a pre-treatment level of TIMP3, a pre-treatment level of BMP2, or a pre-treatment level of secreted uPAR isoform2 in said pre-treatment biological sample.

41. The method of claim 40, wherein said post-treatment level of MMP detected in said post-treatment biological sample is compared to said pre-treatment level of MMP in said pre-treatment biological sample.

42. The method of claim 40, wherein said post-treatment level of TIMP3 detected in said post-treatment biological sample is compared to said pre-treatment level of TIMP3 in said pre-treatment biological sample.

43. The method of claim 40, wherein said post-treatment level of BMP2 detected in said post-treatment biological sample is compared to said pre-treatment level of BMP2 in said pre-treatment biological sample.

44. The method of claim 40, wherein said post-treatment level of secreted uPAR isoform2 detected in said post-treatment biological sample is compared to said pre-treatment level of secreted uPAR isoform2 in said pre-treatment biological sample.a

45. The method of claim 25, wherein said cancer patient has been further treated with an additional anti-cancer agent.

46. The method of claim 45, wherein said additional anti-cancer agent is a ROS inducer.

47. The method of claim 25, wherein said cancer patient has melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer.

48. The method of claim 25, wherein said cancer patient being treated has a metastatic cancer.

49. A method of treating cancer in a subject in need thereof, wherein said subject has a cancer expressing miR-221 and not expressing MDA-7, said method comprising administering to said subject an effective amount of MDA-7.

50. The method of claim 49 wherein said cancer does not express beclin-1.

51. The method of claim 49, further comprising, prior to administering said effective amount of MDA-7:
(iii) obtaining a pre-treatment biological sample from said subject; and
(iv) detecting a pre-treatment level of miR-221 in said pre-treatment biological sample.

52. The method of claim 51, wherein said pre-treatment biological sample is a tumor biopsy.

53. The method of claim 51, wherein said pre-treatment biological sample comprises a circulating tumor cell.

54. The method of claim 51, wherein said detecting comprises performing real-time PCR.

55. The method of claim 51, wherein said detecting comprises performing in situ hybridization.

56. The method of claim 51, wherein said pre-treatment level of miR-221 in said pre-treatment biological sample is compared against a standard control.

57. The method of claim 51, wherein said detecting a pre-treatment level of miR-221 comprises detecting a pre-treatment level of MMP, a pre-treatment level of TIMP3, a pre-treatment level of BMP2, or a pre-treatment level of secreted uPAR isoform2 in said pre-treatment biological sample.

58. The method of claim 57, wherein said pre-treatment level of MMP in said pre-treatment biological sample is compared against a standard control.

59. The method of claim 57, wherein said pre-treatment level of TIMP3 in said pre-treatment biological sample is compared against a standard control.

60. The method of claim 57, wherein said pre-treatment level of BMP2 in said pre-treatment biological sample is compared against a standard control.

61. The method of claim 57, wherein said pre-treatment level of secreted uPAR isoform2 in said pre-treatment biological sample is compared against a standard control.

62. The method of claim 49, wherein administering said effective amount of MDA-7 reverses a multidrug chemoresistance.

63. The method of claim 49, further comprising administering to said subject an additional anti-cancer agent.

64. The method of claim 63, wherein said additional anti-cancer agent is a ROS inducer.

65. The method of claim 49, wherein said cancer is melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer.

66. The method of claim 49, wherein said cancer is a metastatic cancer.

67. A method of treating cancer in a subject in need thereof, wherein said subject has a cancer not expressing beclin-1 and not expressing MDA-7, said method comprising administering to said subject an effective amount of MDA-7.

68. The method of claim 67, wherein said cancer expresses miR-221.

69. The method of claim 67, further comprising, prior to administering said effective amount of MDA-7:
(iii) obtaining a pre-treatment biological sample from said subject; and
(iv) detecting a pre-treatment level of beclin-1 in said pre-treatment biological sample.

70. The method of claim 69, wherein said pre-treatment biological sample is a tumor biopsy.

71. The method of claim 69, wherein said pre-treatment biological sample comprises a circulating tumor cell.

72. The method of claim 69, wherein said detecting comprises performing real-time PCR.

73. The method of claim 69, wherein said detecting comprises performing Western blotting analysis.

74. The method of claim 69, wherein said pre-treatment level of beclin-1 in said pre-treatment biological sample is compared against a standard control.

75. The method of claim 69, wherein said detecting a pre-treatment level of beclin-1 comprises detecting a pre-treatment level of MMP, a pre-treatment level of TIMP3, a pre-treatment level of BMP2, or a pre-treatment level of secreted uPAR isoform 2 in said pre-treatment biological sample.

76. The method of claim 75, wherein said pre-treatment level of MMP in said pre-treatment biological sample is compared against a standard control.

77. The method of claim 75, wherein said pre-treatment level of TIMP3 in said pre-treatment biological sample is compared against a standard control.

78. The method of claim 75, wherein said pre-treatment level of BMP2 in said pre-treatment biological sample is compared against a standard control.

79. The method of claim 75, wherein said pre-treatment level of secreted uPAR isoform2 in said pre-treatment biological sample is compared against a standard control.

80. The method of claim 67, wherein administering said effective amount of MDA-7 reverses a multidrug chemoresistance.

81. The method of claim 67, further comprising administering to said subject an additional anti-cancer agent.

82. The method of claim 81, wherein said additional anti-cancer agent is a ROS inducer.

83. The method of claim 67, wherein said cancer is melanoma, prostate cancer, neuroblastoma, osteosarcoma, renal carcinoma, leukemia, epithelial cancer, pancreatic cancer, glioblastoma, thyroid papillary carcinoma, esophageal squamous cell carcinoma, breast cancer, hepatocellular carcinoma, liver cancer, or lung cancer.

84. The method of claim 67, wherein said cancer is a metastatic cancer.

85. A method of inhibiting cancer-associated angiogenesis in a subject in need thereof, said method comprising administering to said subject an effective amount of MDA-7.

86. A method of treating an autoimmune disease in a subject in need thereof, said method comprising administering to said subject an effective amount of MDA-7.

87. A method of treating an infectious disease in a subject in need thereof, said method comprising administering to said subject an effective amount of MDA-7.

88. A method of treating an inflammatory disease in a subject in need thereof, said method comprising administering to said subject an effective amount of MDA-7.

89. A method of treating a cardiovascular disease in a subject in need thereof, said method comprising administering to said subject an effective amount of MDA-7.
